SEQUENCE LISTING (17 sequences)

SEQ ID NO: 1 - DNA, 20 bp, synthetic construct
  source 1..20: mol_type = other DNA
aactccatcgaactaatgcc

SEQ ID NO: 2 - skipped

SEQ ID NO: 3 - DNA, 16 bp, synthetic construct
  source 1..16: mol_type = other DNA
tagcaagagagtctta

SEQ ID NO: 4 - DNA, 20 bp, synthetic construct
  source 1..20: mol_type = other DNA
cattcaccgtggaagcagtc

SEQ ID NO: 5 - DNA, 20 bp, synthetic construct
  source 1..20: mol_type = other DNA
gtaagtggcaccttcgtcag

SEQ ID NO: 6 - DNA, 16 bp, synthetic construct
  source 1..16: mol_type = other DNA
taatcccacgctgtga

SEQ ID NO: 7 - DNA, 20 bp, synthetic construct
  source 1..20: mol_type = other DNA
attccctcgtaacggagaag

SEQ ID NO: 8 - DNA, 32 bp, synthetic construct
  source 1..32: mol_type = other DNA
cagaatcattcaccgtggaagcagtcattagg

SEQ ID NO: 9 - DNA, 40 bp, synthetic construct
  source 1..40: mol_type = other DNA
ccgcccttcgcctacattatggacttcgacataattgact

SEQ ID NO: 10 - DNA, 41 bp, synthetic construct
  source 1..41: mol_type = other DNA
ataatgtaggcgaagggcggnnnnnnnnnngccttgtctta

SEQ ID NO: 11 - DNA, 52 bp, synthetic construct
  source 1..52: mol_type = other DNA
ggattactgacagaagagaagcttactaagactggtcctacgagacatcggt

SEQ ID NO: 12 - DNA, 20 bp, synthetic construct
  source 1..20: mol_type = other DNA
gtaagcttctcttctgtcag

SEQ ID NO: 13 - DNA, 57 bp, synthetic construct
  source 1..57: mol_type = other DNA
taatccgcatgnnnnnnnnnntggtcctacgagacatcggtatcaggttcacagcag

SEQ ID NO: 14 - DNA, 16 bp, synthetic construct
  source 1..16: mol_type = other DNA
ctgctgtgaacctgat

SEQ ID NO: 15 - DNA, 20 bp, synthetic construct
  source 1..20: mol_type = other DNA
accgatgtctcgtaggacca

SEQ ID NO: 16 - DNA, 11 bp, synthetic construct
  source 1..11: mol_type = other DNA
taagacaaggc

SEQ ID NO: 17 - DNA, 11 bp, synthetic construct
  source 1..11: mol_type = other DNA
catgcggatta

(19) United States
(12) Patent Application Publication
(10) Pub. No.: US 2026/0337679 A1
(43) Pub. Date: Oct. 29, 2026

(54) METHODS FOR COMBINATORIAL IMMUNOSEQUENCING

(71) Applicant: MESO SCALE TECHNOLOGIES, LLC., Rockville, MD (US)

(72) Inventors: David ROUTENBERG, Gaithersburg, MD (US); Lucie HEBERT, Bethesda, MD (US); Evan GIZZIE, Washington, DC (US); Deepak NANJAPPA, Ijamsville, MD (US)

(21) Appl. No.: 19/376,362

(22) Filed: Oct. 31, 2025

Related U.S. Application Data
(60) Provisional application No. 63/715,097, filed on Nov. 1, 2024.

(51) Int. Cl. C12Q 1/6874; C12Q 1/6886

(52) U.S. Cl. CPC C12Q 1/6874; C12Q 1/6886; C12Q 2600/106; C12Q 2600/158; G01N 2800/52

(57) ABSTRACT

The disclosure relates to methods, compositions, and kits for detecting and analyzing surface marker displaying agents (SMDAs). The disclosure further relates to methods for detecting, diagnosing, monitoring response to treatment or recurrence, or predicting overall survival, progression-free survival or duration of response in a subject suspected of having colorectal cancer (CRC) comprising performing one or more assays on at least one biological sample from the subject to detect one or more specific populations of EVs.

This invention was made with government support under grant number DK133861 awarded by the National Institutes of Health and grant number TR002886 awarded by the National Institutes of Health. The government has certain rights in the invention.

### FIELD OF THE DISCLOSURE

The disclosure relates to methods, compositions, and kits for the analysis, specific detection, and isolation of surface marker displaying agents (SMDAs) such as extracellular vesicles (EVs) and/or their contents by targeting at least two surface markers. The disclosure further relates to methods for detecting, diagnosing, monitoring response to treatment or recurrence, or predicting overall survival, progression-free survival or duration of response in a subject suspected of having colorectal cancer (CRC) comprising performing one or more assays on at least one biological sample from the subject to determine surface markers specific to populations of EVs.

### REFERENCE TO ELECTRONIC SEQUENCE LISTING

The application contains a Sequence Listing which has been submitted electronically in .XML format and is hereby incorporated by reference in its entirety. Said .XML copy, created on Oct. 29, 2025, is named “0076-0097WO1.xml” and is 15,256 bytes in size. The sequence listing contained in this .XML file is part of the specification and is hereby incorporated by reference herein in its entirety.

### BACKGROUND

Surface marker displaying agents (SMDAs) include cells, viruses and viral particles, cellular organelles, and vesicles, including extracellular vesicles (EVs) and exosomes. In addition to the potentially biologically relevant information displayed on the surface of SMDAs, SMDAs may encapsulate biologically relevant materials or components.

EVs are a diverse group of cell-secreted membrane vesicles implicated in a wide variety of physiological and pathological processes, many of which are only beginning to be understood. These include immune regulation, antigen presentation, tumor progression and metastasis, modulation of inflammation, stem cell regulation, neuronal development and regeneration, and cell-to-cell transfer of pathogenic proteins and nucleic acids. EVs are secreted from nearly all cell types through multiple mechanisms including the fusion of specific endosomal compartments called multivesicular bodies (MVB) with the plasma membrane and by budding/shedding directly from the plasma membrane. EVs are present in nearly all body fluids including blood, urine, cerebral spinal fluid, and saliva, and are secreted by most in vitro cultured cells as well. Because of the EV formation mechanisms, EVs contain specific lipids, membrane proteins, and internalized proteins, nucleic acids and metabolites derived from their cells of origin and are thus a rich source of potential biomarkers.

It is now accepted that EVs have numerous roles in inter-cellular communication, facilitated by the transfer of EV cargo to recipient cells following uptake, or by the interaction of EV surface proteins with cellular receptors. EVs are also emerging as useful indicators of disease. EV secretion is involved in the maintenance of normal physiological functions and is linked to numerous disease states, including certain cancers, cardiovascular disease, neurological and immunological disorders. Methods for detecting and isolating EVs are described, for example, in international patent application publication numbers WO2019222708, US20210389304, WO2020086751, US20210382043A1, WO2022051481, US20230349920A1, and WO2023212315, and U.S. application Ser. No. 18/860,884, each of which is incorporated by reference in its entirety.

Colorectal cancer (CRC) is the third most common cancer worldwide, accounting for almost 10% of all cases, and is the second leading cause of cancer death in the United States. CRC recurrence rate averages 25%, and follow-up after curative treatment remains a complex challenge for the healthcare system, in terms of diagnostics, treatment and monetary costs. In general, the survival of patients with advanced-stage cancers is lower than that of patients with early-stage cancers, although some survival paradox has been reported between stage IIB/C and stage IIIA. Other prognostic markers are used as indicators of the appropriate therapeutic strategy, but are excluded from the classification, including the histological grade, RAS/BRAF mutation, and plasma carcinoembryonic antigen (CEA) levels.

Assessing the composition of EVs generally requires isolating a pure population of EVs and separating it from non-EV associated factors. Some demonstrations of this idea have focused on enriching EVs from plasma or serum based on immunoaffinity capture of specific EV surface proteins and measuring disease-associated proteins within the enriched EV population.

Despite its utility, this method has significant fundamental and technical drawbacks. Fundamentally, the use of a single marker for EV isolation presents a great challenge. Most surface proteins are expressed on a variety of cell types; thus, multiple markers are usually needed to define a specific cell population. This is often apparent in flow cytometry, wherein multiple markers are usually employed despite the benefit of a predefined input cell population (e.g. PBMCs, or cultured cells). When isolating EVs from blood, nearly all cell types of the organism may be represented within the EV population, increasing the challenge of identifying a single marker specific for EVs from one cell type. Technical challenges of the existing approach are illustrated by the dramatic differences in levels of circulating L1CAM+ EVs and associated cargo molecules (e.g. Tau) reported by multiple groups using nearly identical protocols. This variability, which likely stems from minor variations in protocols from lab to lab (e.g. wash or mixing steps) speaks to the need for protocol standardization and simplification.

### SUMMARY OF THE DISCLOSURE

In some aspects, the present disclosure provides a method of determining surface markers of a surface marker displaying agent (SMDA) comprising: (A) contacting the SMDA with (i) a capture reagent bound to a surface, (ii) a plurality of binding reagents, and (iii) an oligonucleotide insert, wherein the plurality of binding reagents comprises: a) a first binding reagent comprising a first detection sequence that comprises (i) a first hybridization sequence, wherein at least a portion of the first hybridization sequence is complexed with a first blocker oligonucleotide, (ii) a first primer site, and (iii) a first barcode sequence; b) a second binding reagent comprising a second detection sequence that comprises (i) a second hybridization sequence, (ii) a third hybridization sequence comprising a second barcode sequence, and (iii) a fourth hybridization sequence; and c) a third binding reagent comprising a third detection sequence that comprises (i) a fifth hybridization sequence, wherein at least a portion of the fifth hybridization sequence is complexed with a second blocker oligonucleotide, (ii) a second primer site, and (iii) a third barcode sequence, wherein the first hybridization sequence and the second hybridization sequence are complementary; wherein the fourth hybridization sequence and the fifth hybridization sequence are complementary; wherein the third hybridization sequence is complementary to the oligonucleotide insert sequence; (B) generating an output oligonucleotide by ligating the hybridized first detection sequence to the hybridized oligonucleotide insert, and ligating the hybridized oligonucleotide insert to the hybridized third detection sequence to form a single-strand output oligonucleotide, wherein when at least three binding reagents bind to three surface markers of the SMDA, the output oligonucleotide generated comprises the first barcode sequence, the second barcode sequence and the third barcode sequence; and (C) sequencing the output oligonucleotide to identify the three surface markers of the SMDA. In some aspects, the capture reagent is bound to a first surface, and the first, second, and third binding reagent are bound to a second surface. In some aspects, the capture reagent and the third binding reagent are bound to the same surface. In some aspects, the capture reagent and the third binding reagent are independently bound to the same surface. In some aspects, the capture reagent and the third binding reagent are bound to separate surfaces.

In some aspects, the present disclosure provides a method of determining surface markers of a surface marker displaying agent (SMDA) comprising: (A) contacting the SMDA with (i) a capture reagent bound to a surface, (ii) a plurality of binding reagents, and (iii) an oligonucleotide insert, wherein the plurality of binding reagents comprises: a) a first binding reagent comprising a first conjugating oligonucleotide and a first detection sequence, the first detection sequence comprising: (i) a first hybridization sequence, (ii) a first barcode sequence, (iii) a first primer site, and (iv) a first connecting sequence complementary to the first conjugating oligonucleotide; b) a second binding reagent comprising a second conjugating oligonucleotide and a second detection sequence, the second detection sequence comprising: (i) a second hybridization sequence, (ii) a third hybridization sequence comprising a second barcode sequence, (iii) a fourth hybridization sequence, (iv) and a second connecting sequence complementary to the second conjugating oligonucleotide; and c) a third binding reagent comprising a third conjugating oligonucleotide and a third detection sequence, the third detection sequence comprising: (i) a fifth hybridization sequence, (ii) a third barcode sequence, (iii) a second primer site, (iv) a third connecting sequence complementary to the third conjugating oligonucleotide, and (v) a third primer site, wherein the first hybridization sequence and the second hybridization sequence are complementary; wherein the fourth hybridization sequence and the fifth hybridization sequence are complementary; wherein the third hybridization sequence is complementary to the oligonucleotide insert sequence; (B) generating an output oligonucleotide by ligating the hybridized first detection sequence to the hybridized oligonucleotide insert, and ligating the hybridized oligonucleotide insert to the hybridized third detection sequence to form a single-strand output oligonucleotide, wherein when at least three binding reagents bind to three surface markers of the SMDA, the output oligonucleotide generated comprises the first barcode sequence, the second barcode sequence and the third barcode sequence; and (C) sequencing the output oligonucleotide to identify the three surface markers of the SMDA.

In some aspects, the present disclosure provides a method of determining surface markers of a surface marker displaying agent (SMDA), comprising: (A) contacting a sample comprising a SMDA with: (a) an anchoring reagent hybridized to a first detection oligonucleotide, wherein the first detection oligonucleotide comprises a first unique barcode sequence and a first primer site; (b) a first binding reagent hybridized to a second detection oligonucleotide, wherein the second detection oligonucleotide comprises a second unique barcode sequence, wherein the first detection oligonucleotide and the second detection oligonucleotide comprise complementary nucleotide sequences; (c) a second binding reagent hybridized to a third detection oligonucleotide, wherein the third detection oligonucleotide comprises a third unique barcode sequence, a second primer site, and a third primer site, wherein the second detection oligonucleotide and the third detection oligonucleotide comprise complementary nucleotide sequences; (d) a capture reagent; and (e) an oligonucleotide insert complementary to the second detection oligonucleotide, wherein if: (i) the SMDA binds to the capture reagent, the first and second binding reagent; and (ii) the first detection oligonucleotide hybridizes to the second detection oligonucleotide, the second detection oligonucleotide hybridizes to the third detection oligonucleotide, and the oligonucleotide insert hybridizes to the second detection oligonucleotide, then a single-strand output oligonucleotide is generated that comprises the first, second, and third unique barcode sequences; (B) generating a double-strand output oligonucleotide from the single-strand output oligonucleotide by binding a primer complementary to the third primer site and extending the primer to form a second strand complementary to the single-strand output oligonucleotide; (C) amplifying the double-strand output oligonucleotide; and (D) sequencing the amplified output oligonucleotide to identify the first, second, and third unique barcode oligonucleotide sequences, thereby determining at least three unique surface markers of the SMDA. In some aspects, the capture reagent and the anchoring reagent are bound to the same surface. In some aspects, the capture reagent and the anchoring reagent are independently bound to the same surface. In some aspects, the capture reagent and the anchoring reagent are bound to separate surfaces.

In some embodiments, the present disclosure provides a method of determining surface markers of a surface marker displaying agent (SMDA), comprising: (A) contacting a sample comprising an SMDA with: (a) a capture reagent that binds to a first surface marker of the SMDA; (b) an anchoring reagent comprising an anchoring oligonucleotide; (c) a first detection oligonucleotide comprising: (i) a first primer site; (ii) a first barcode sequence; (iii) a first blocker complement sequence; and (iv) a first hybridization sequence, wherein the first detection oligonucleotide and the anchoring oligonucleotide comprise complementary nucleotide sequences; (d) a first binding reagent that binds to a second surface marker of the SMDA, wherein the first binding reagent comprises a first conjugating oligonucleotide; (e) a second detection oligonucleotide comprising: (i) a second hybridization sequence; (ii) a third hybridization sequence comprising a second barcode sequence; and (iii) a fourth hybridization sequence; (f) a second binding reagent that binds to a third surface marker of the SMDA, wherein the second binding reagent comprises a second conjugating oligonucleotide; (g) a third detection oligonucleotide comprising: (i) a fifth hybridization sequence; (ii) a second blocker complement sequence; (iii) a third barcode sequence; (iv) a second primer site; and (iv) a third primer site; and (h) an oligonucleotide insert, wherein the first hybridization sequence and the second hybridization sequence are complementary; wherein the fourth hybridization sequence and the fifth hybridization sequence are complementary; and wherein the third hybridization sequence is complementary to the oligonucleotide insert; B) generating an output oligonucleotide by ligating the hybridized first detection oligonucleotide to the hybridized oligonucleotide insert, and ligating the hybridized oligonucleotide insert to the hybridized third detection oligonucleotide to form an output oligonucleotide, wherein the output oligonucleotide generated comprises the first barcode sequence, the second barcode sequence, and the third barcode sequence; C) amplifying the output oligonucleotide; and D) sequencing the output oligonucleotide to identify the first, second, and third barcode sequences, thereby determining the surface markers of the SMDA.

In some embodiments, the present disclosure provides a method of determining surface markers of a surface marker displaying agent (SMDA), comprising: (A) contacting the SMDA with: (a) a capture reagent that binds to a first surface marker of the SMDA; (b) an anchoring reagent comprising a first hybridization sequence; (c) a reverse oligonucleotide comprising: (i) a first barcode sequence; (ii) a second hybridization sequence; and (iii) a third hybridization sequence, wherein the first hybridization sequence and the second hybridization sequence are complementary; (d) a binding reagent that binds to a second surface marker of the SMDA, wherein the binding reagent comprises a fourth hybridization sequence; and (e) a forward oligonucleotide comprising: (i) a fifth hybridization sequence; (ii) a second barcode sequence; and (iii) a sixth hybridization sequence, wherein the third hybridizations sequence and the sixth hybridization sequence are complementary, and wherein the fourth hybridization sequence and the fifth hybridization sequence are complementary; B) generating an output oligonucleotide by extending the hybridized reverse oligonucleotide and the hybridized forward oligonucleotide to form an output oligonucleotide, wherein the output oligonucleotide generated comprises the first barcode sequence and the second barcode sequence; C) amplifying the output oligonucleotide; and D) sequencing the output oligonucleotide to identify the first and second barcode sequences, thereby determining the surface markers of the SMDA.

In some aspects, the present disclosure provides a method for detecting colorectal tumor-derived surface marker displaying agent (SMDA) in a sample from a subject suspected of having a colorectal tumor or suspected of having colorectal cancer (CRC), comprising detecting at least two CRC-related markers on the SMDA in the sample.

In some aspects, the present disclosure provides a method for determining eligibility of a subject to participate in a clinical trial of a therapeutic drug for preventing or delaying colorectal cancer (CRC), comprising: (a) obtaining a measurement of at least two CRC-related surface marker levels on colorectal tumor-derived surface marker displaying agents (SMDAs) in a biological sample from the subject; and (b) determining the eligibility of the subject for the clinical trial based on the measurement of the at least two CRC-related surface marker levels.

In some aspects, the present disclosure provides a method for conducting a clinical trial of a therapeutic drug or intervention for colorectal cancer (CRC), comprising: (a) obtaining a measurement of at least two CRC-related surface marker levels on colorectal tumor-derived surface marker displaying agents (SMDAs) in a biological sample from a subject; (b) determining eligibility of the subject for the clinical trial based on the measurement of the at least two CRC-related surface marker levels; and (c) administering the therapeutic drug to the subject.

In some aspects, the present disclosure provides a method for distinguishing a subject afflicted with colorectal cancer (CRC) from an individual not afflicted with CRC, comprising: (a) obtaining a measurement of at least two CRC-related surface marker levels on colorectal tumor-derived surface marker displaying agents (SMDAs) in a biological sample from the subject; and (b) identifying, based on the measurement of the at least two CRC-related surface marker levels, the subject as (i) afflicted with CRC or (ii) not afflicted with CRC.

In some aspects, the present disclosure provides a method for treating colorectal cancer (CRC) in a subject in need thereof, comprising: (a) obtaining a measurement of at least two CRC-related surface marker levels on colorectal tumor-derived surface marker displaying agents (SMDAs) in a biological sample from the subject, wherein the measurement is obtained prior to administration of a treatment for CRC, (b) determining based on the measurement of the at least two CRC-related surface marker levels, that the subject is afflicted with CRC, and (c) administering a treatment regimen for CRC to the subject.

In some aspects, the present disclosure provides a method for monitoring response to treatment for colorectal cancer (CRC) in a subject, the method comprising: (a) determining, based on a first measurement of at least two CRC-related marker levels on colorectal tumor-derived surface marker displaying agents (SMDAs) in a biological sample from the subject, wherein the first measurement is obtained prior to administration of a treatment regimen for CRC and a second measurement of the at least two CRC-related marker levels in the subject at one or more time points after administration of the treatment regimen for CRC has been initiated, that the subject is responding positively to the CRC treatment regimen, and (b) continuing to administer the treatment regimen for CRC to the subject.

In some aspects, the present disclosure provides a method for identifying colorectal cancer (CRC) in a human, the method comprising: obtaining measured levels of at least two CRC-related marker levels on colorectal tumor-derived surface marker displaying agents (SMDAs) in a biological sample suspected of containing SMDAs from the human, wherein the sample is selected from the group consisting of whole blood, serum, plasma, and combinations thereof, and wherein the levels of the at least two CRC-related markers are obtained by a multimarker immunoassay comprising contacting the sample with a first oligonucleotide-conjugated capture entity that binds a first CRC-related surface marker of the SMDA, a second oligonucleotide-conjugated splint entity that binds a second CRC-related surface marker of the SMDA, a third oligonucleotide-conjugated staple entity that binds a common marker or a third CRC-related surface marker of the SMDA, and a surface.

In some aspects, the CRC-related surface markers comprise any combination selected from: CD73, CD324, CD325, CD326, CD13, CD66e, CD66a, CD10, CD31, CD36, CD141, CD14, and CD54.

In some aspects, the present disclosure provides a kit for determining surface markers of a surface marker displaying agent (SMDA), for identifying SMDAs that harbor combinations of surface markers, for detecting populations of SMDAs having certain surface markers, and/or for detecting or quantifying multiple populations of SMDAs where each population has a specific set of surface markers, the kit comprising, in one or more vials, containers, or compartments: a capture reagent, at least three unique binding reagents and an oligonucleotide insert, and at least two blocker oligonucleotides. In some aspects, each unique binding reagent comprises a detection sequence comprising a unique barcode oligonucleotide sequence. In some aspects, the at least three unique binding reagents comprises: a) a first binding reagent comprising a first detection sequence that comprises a (i) first hybridization sequence, wherein at least a portion of the first hybridization sequence is complexed with a first blocker oligonucleotide, and (ii) a first primer site; b) a second binding reagent comprising a second detection sequence that comprises (i) a second hybridization sequence, (ii) a third hybridization sequence, and (iii) a fourth hybridization sequence; and c) a third binding reagent comprising a third detection sequence that comprises a fifth hybridization sequence, wherein at least a portion of the fifth hybridization sequence is complexed with a second blocker oligonucleotide, and (ii) a second primer site. In some aspects, the first hybridization sequence and the second hybridization sequence are complementary. In some aspects, the fourth hybridization sequence and the fifth hybridization sequence are complementary. In some aspects, the third hybridization sequence is complementary to the oligonucleotide insert sequence. In some aspects, the kit further comprises a hairpin blocker to inhibit inter-EV ligation.

In some aspects, the present disclosure provides a kit for determining surface markers of a surface marker displaying agent (SMDA), for identifying SMDAs that harbor combinations of surface markers, for detecting populations of SMDAs having certain surface markers, and/or for detecting or quantifying multiple populations of SMDAs where each population has a specific set of surface markers, the kit comprising, in one or more vials, containers, or compartments: (a) a capture reagent; (b) an anchoring reagent comprising an anchoring oligonucleotide; (c) a first binding reagent comprising a first conjugating oligonucleotide; (d) a second binding reagent comprising a third conjugating oligonucleotide; (e) a first detection oligonucleotide comprising: (i) a first primer site; (ii) a first barcode sequence; (iii) a first blocker complement sequence; and (iv) a first hybridization sequence, wherein the first detection oligonucleotide and the anchoring oligonucleotide comprise complementary nucleotide sequences; (f) a second detection oligonucleotide comprising: (i) a second hybridization sequence; (ii) a third hybridization sequence comprising a second barcode sequence; and (iii) a fourth hybridization sequence; (g) a third detection oligonucleotide comprising: (i) a fifth hybridization sequence; (ii) a second blocker complement sequence; (iii) a third barcode sequence; (iv) a second primer site; and (iv) a third primer site; and (h) an oligonucleotide insert, wherein the first hybridization sequence and the second hybridization sequence are complementary; wherein the fourth hybridization sequence and the fifth hybridization sequence are complementary; and wherein the third hybridization sequence is complementary to the oligonucleotide insert

In some aspects, the present disclosure provides a construct comprising: (a) a capture reagent, (b) at least three unique binding reagents and an oligonucleotide insert, and (c) at least two blocker oligonucleotides. In some aspects, each unique binding reagent comprises a detection sequence comprising a unique barcode oligonucleotide sequence. In some aspects, the at least three binding reagents comprises: a) a first binding reagent comprising a first detection sequence that comprises a (i) first hybridization sequence, wherein at least a portion of the first hybridization sequence is complexed with a first blocker oligonucleotide, and (ii) a first primer site; b) a second binding reagent comprising a second detection sequence that comprises (i) a second hybridization sequence, (ii) a third hybridization sequence, and (iii) a fourth hybridization sequence; and c) a third binding reagent comprising a third detection sequence that comprises a fifth hybridization sequence, wherein at least a portion of the fifth hybridization sequence is complexed with a second blocker oligonucleotide, and (ii) a second primer site. In some aspects, the first hybridization sequence and the second hybridization sequence are complementary. In some aspects, the fourth hybridization sequence and the fifth hybridization sequence are complementary. In some aspects, the third hybridization sequence is complementary to the oligonucleotide insert sequence. In some aspects, the third binding reagent further comprises a third primer site.

### BRIEF DESCRIPTION OF THE DRAWINGS

The following drawings form part of the present specification and are included to further demonstrate exemplary embodiments of certain aspects of the present disclosure.

FIG. 1A and FIG. 1B show a schematic overview of an embodiment of a proximity ligation-ligation (PLL) method assay, with a first detection sequence (“5′oligo”); second detection sequence (“splint oligo”); third detection sequence (“3′ oligo”); first hybridization sequence (5′ “Splint Hyb Region”); and fifth hybridization sequence (3′ “Splint Hyb Region”). FIG. 1C shows exemplary sequences used in the PLL assay, including the second detection sequence, or “splint” (SEQ ID NO: 8), and the output oligonucleotide comprising: a first primer site (“For. Primer”) (SEQ ID NO: 1); two UMI oligonucleotides (SEQ ID NO: 2); a first barcode sequence (“BC1”) (SEQ ID NO: 3); a second barcode sequence (“BC2”) (SEQ ID NO: 4); a reverse complement second barcode sequence (“BC2-RC”) (SEQ ID NO: 5); a third barcode sequence (BC3) (SEQ ID NO: 6), and a second primer site (“Rev. Primer”) (SEQ ID NO: 7).

FIG. 2 shows an alternative embodiment of the oligonucleotide construct in FIG. 1, wherein the first detection sequence (“5′ oligo”) comprises a first blocker complement sequence between the first barcode sequence and the first hybridization sequence (5′ “Splint Hyb Region”), and the third detection sequence (“3′ oligo”) comprises a second blocker complement sequence between the fifth hybridization sequence (3′ “Splint Hyb Region”) and the third barcode sequence. In embodiments, the first blocker oligonucleotide is complementary to the first blocker complement sequence and the first hybridization sequence. In embodiments, the second blocker oligonucleotide is complementary to the second blocker complement sequence and the fifth hybridization sequence.

FIG. 3 shows the first 40 most significantly enriched phenotype bins, sorted by p-value, for CRC samples from a first population of subjects with refractory CRC vs. Control samples, and a volcano plot showing enrichment of the phenotype bins.

FIG. 4 shows the first 40 most significantly enriched phenotype bins, sorted by p-value, for CRC samples from a second population of subjects with refractory CRC vs. Control samples, and a volcano plot showing enrichment of the phenotype bins.

FIG. 5 shows the first 40 most significantly enriched phenotype bins, sorted by p-value, for CRC samples from a population of subjects with stage IV CRC vs. Control samples, and a volcano plot showing enrichment of the phenotype bins.

FIG. 6 shows tables of the survival phenotype bins that were most significantly increased in CRC samples from a first population of subjects with refractory CRC vs. Control samples, from a survival analysis using Cox regression for overall survival (OS) and progression free survival (PFS). The bins are sorted by p-values.

FIG. 7 shows tables of the survival phenotype bins that were most significantly increased in CRC samples from a second population of subjects with refractory CRC vs. Control samples, from a survival analysis using Cox regression for overall survival (OS) and progression free survival (PFS). The bins are sorted by p-values.

FIG. 8 shows a table of the phenotype bins that were most significantly increased in Breast Cancer samples vs Control samples using Mann-Whitney tests. The bins are sorted by p-values.

FIG. 9 shows a table of the survival phenotype bins that were most significantly increased in Gastric/Esophageal Cancer samples vs Control samples using Mann-Whitney tests. The bins are sorted by p-values.

FIG. 10A, FIG. 10B, and FIG. 10C show the quantitative performance of the PLL assays where the signal for several bins were compared to ECL assays previously run on the same samples from Example 1. FIG. 10A shows the correlation of results between PLL and ECL using an intact EV sandwich assay for CEA+ EVs. FIG. 10B shows the correlation of results between PLL and ECL using an intact EV sandwich assay for CD73+ EVs. FIG. 10C shows the correlation of results between PLL and ECL using an intact EV assay for CEA+CD73+ EVs.

FIG. 11 shows a schematic of a PLL assay with Strand Displacement (PLLSD). The full-length output oligonucleotide strand is displaced by strand-displacement amplification (SDA).

FIG. 12 shows a schematic of a PLLSD assay using clonal beads for capture. In this embodiment, the capture reagent on the bead surface is specific to a CRC surface marker. In this embodiment, the third binding reagent is on the bead surface.

FIG. 13 shows a schematic of a PLLSD assay with the use of a hairpin blocker. After ligation of the output oligonucleotide strand, the remaining unligated oligos are terminated by adding hairpin oligos that hybridize to the free ends of the oligos and ligate to eliminate the possibility of inter-EV reactions.

FIG. 14 shows a detailed schematic illustrating an embodiment of the PLLSD assay using clonal beads to capture EVs including the anchoring reagent (“Capture Anchor”); first detection oligonucleotide (“5P Oligo”); second detection oligonucleotide (“Splint Oligo”); and third detection oligonucleotide (“3P Oligo”).

FIG. 15 shows a volcano plot of enriched phenotype bins measured in breast cancer (BC) samples versus healthy control samples according to the PLLSD configuration of FIG. 1A, FIG. 1B, and FIG. 1C.

FIG. 16 shows a gel image corresponding to the qPCR Ct amplicons using the PLLSD configuration shown in FIG. 11 under different conditions as described in Example 3.

FIG. 17 shows a schematic illustrating the transition from the long version to the shortened version of PLLSD oligonucleotides as described in Example 3, including the anchoring oligonucleotide (“Biotin-Anchor”); first detection oligonucleotide (“5′ Prime”); second detection oligonucleotide (“Splint”); third detection oligonucleotide (“3′ Prime”); first/second conjugating oligonucleotide (“Antibody-Anchor”); first primer site (“FW Primer Region”); second primer site (“RV Primer Region”); first hybridization sequence (5′ “Splint Hyb Region”); and fifth hybridization sequence (3′ “Splint Hyb Region”).

FIG. 18 shows exemplary oligonucleotide sequences of the shortened version of the PLLSD assay, including the anchoring oligonucleotide (“PLLSD Capture Anchor”) (SEQ ID NO: 9), first detection oligonucleotide (“5Prime”) (SEQ ID NO: 10), second detection oligonucleotide (“Splint”) (SEQ ID NO: 11), oligonucleotide insert (“Insert”) (SEQ ID NO: 12), third detection oligonucleotide (“3Prime”) (SEQ ID NO: 13), strand-displacement primer (SEQ ID NO:14), first and second conjugating oligonucleotide (“PLLSD Detector Anchor”) (SEQ ID NO: 15), first blocker oligonucleotide (“Blocker 5Prime”) (SEQ ID NO: 16), and second blocker oligonucleotide (“Blocker 3Prime”) (SEQ ID NO: 17).

FIG. 19 shows a gel image corresponding to the qPCR Ct amplicons generated from the shortened version of the PLLSD assay under different conditions as described in Example 3.

FIGS. 20A and 20B show schematics illustrating an embodiment of the use of a two-antibody assay, “Proximity-Extension assay with Strand Displacement” (PESD), including the anchoring oligonucleotide (“Reverse Anchor”); first hybridization sequence (“H1”); second hybridization sequence (“H2”); third hybridization sequence (“H3”); fourth hybridization sequence (“Forward-Anchor” or “H4”); fifth hybridization sequence (“H5”); and sixth hybridization sequence (“H6”).

FIG. 21 shows logarithmic plots of measured PESD signal against EV concentration, as described in Example 4, using common EV markers CD9, CD81 and CD63 as detectors, in combination with CD4 and CD81 as capture markers.

FIG. 22A shows a schematic illustrating an embodiment with the use of an PLLSD assay configuration in which the existing first conjugating oligonucleotide and second conjugating oligonucleotide (“Antibody-Anchor Detector”) of the three-antibody PLLSD assay as exemplified in FIG. 14 and FIG. 17 are substituted with the fourth hybridization sequence of the two-antibody PESD assay (“PESD FW Anchor—Antibody-Anchor”). FIG. 22B shows a schematic illustrating an alternative embodiment of the PLLSD assay configuration comprising an adaptor oligonucleotide comprising two sequences, the first sequence complementary to the PLLSD first detection oligonucleotide (“5′ Prime”) and/or third detection oligonucleotide (“3′ Prime”) and the second sequence complementary to the PESD fourth hybridization sequence (“PESD FW Anchor”). FIGS. 22A and 22B also illustrate the second detection oligonucleotide (“Splint”); first and second adaptor oligonucleotides (“PLLSD-PESD Detect Anchor Adaptor”); first hybridization sequence (5′ “Splint Hyb Region”); and fifth hybridization sequence (3′ “Splint Hyb Region”).

### DETAILED DESCRIPTION OF THE DISCLOSURE

Unless otherwise defined herein, scientific and technical terms used in the present disclosure shall have the meanings that are commonly understood by one of ordinary skill in the art.

Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.” “And/or” where used herein is to be taken as specific disclosure of each of the specified features of components with or without the other. Thus, the term “and/or” as used in a phrase such as “A and/or B” herein is intended to include “A and B,” “A or B,” “A” (alone), and “B” (alone).

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or “about” can mean rounded to the nearest significant digit.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

As used herein, “between” is a range inclusive of the ends of the range. For example, a number between x and y explicitly includes the numbers x and y and any numbers that fall within x and y.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)2, Fab, Fab′, and F(ab′)2, F(ab1)2, Fv, dAb, and Fd fragments, diabodies, and antibody-related polypeptides, so long as they exhibit the desired biological activity or function.

As used herein, the term “same”, “substantially the same” or “similar”, are used interchangeably and encompass elements identical in about every detail. In embodiments they are about 80% to about 100% identical. In embodiments, they are about 90% identical. In embodiments, they are about 95% identical. In embodiments, they are about 100% identical.

### Introduction

There has been growing interest in measuring EVs, a type of SMDA, as biomarkers for characterizing biological processes in cell lines or model organisms, or as diagnostic indicators of disease processes in people. Since it is the surface EV protein composition that will largely dictate their biological behavior, high-throughput single EV profiling methods are needed to better define EV subpopulations. Current immunoassays for measuring intact EVs are based on the presence of one or more EV surface proteins. These methods enable quantitative and qualitative comparisons of the number and character of EVs in complex biological samples.

One existing method for screening EVs is a combinatorial screening method based on a proximity extension ligation (PEL) reaction that only produces signal (amplifiable DNA with 3 barcodes) when three antibodies are bound to the same EV. See, e.g., PCT patent application publication nos. WO2020086751 and WO2022051481. While this reaction has high specificity, it would be desirable to increase the efficiency of converting antibody triplets into full length DNA, thus reducing bottlenecks for sequencing analysis.

The present disclosure provides methods of isolating and/or characterizing SMDAs. SMDAs can be naturally-occurring, partially synthetic, or fully synthetic. In embodiments, a SMDA is a biologically relevant material or component. In general, a SMDA comprises a surface, typically a lipid bilayer, membrane, cell wall, or envelope, on which one or more markers are displayed. In embodiments, the SMDA encapsulates components such as, e.g., proteins, nucleic acids, lipids, carbohydrates, small molecules such as hormones, cofactors, vitamins, minerals, salts, metals, metal-containing compounds, or combination thereof. Examples of SMDAs include cells (including prokaryotic cells such as bacterial cells or archaeal cells; eukaryotic cells such as mammalian cells, insect cells, or plant cells); viruses and viral particles; cellular organelles such as nucleus, endoplasmic reticulum, Golgi apparatus, mitochondria, vacuoles, or chloroplast; vesicles such as lysosome, endosome, peroxisome, and liposome; and EVs or exosomes. Although the present specification may refer to EVs in certain embodiments, the disclosure contemplates that such aspects also apply to any SMDA provided herein without limitation.

A variety of analytical methods have been used to characterize EVs including, most commonly, immunoassays (Western blotting, flow cytometry, sandwich immunoassays), electron microscopy, mass spectrometry, PCR and sequencing, and nanoparticle tracking. One of the most significant limitations to characterizing EVs has been the difficulty of separating EVs from the other components in complex biofluids.

EV isolation, enrichment, and purification have been the subject of extensive discussion and publication yet there is still not one universally-accepted method. Ultracentrifugation, ultrafiltration, size-exclusion chromatography, and immuno-affinity based methods all have their strengths and shortcomings. Each must be applied in the appropriate situation with full recognition of the potential for introducing bias or allowing contamination by non-EV components of the sample. Analytical methods that avoid pre-purification steps are advantageous as they introduce no bias in the EV population subject to analysis; however, they have the highest risk of negative effects due to non-EV related molecular interactions and artifacts.

The inventors have developed a surprisingly effective and highly specific method of detecting and isolating SMDAs of interest from samples. In embodiments, and by way of example, the method indirectly attaches an SMDA to a surface using at least two, and, in some cases, at least three, separate SMDA surface markers. In embodiments, first, an SMDA is indirectly attached to a surface using an SMDA surface marker, then a second indirect attachment point is formed by way of a second SMDA surface marker. In embodiments, following removal of unwanted components, the first indirect attachment point is broken, leaving the SMDA indirectly attached to the surface by only the second surface marker. In this way, SMDAs not having either surface marker, or SMDAs having only the first surface marker, are also released from the surface, leaving only SMDAs having both markers. In embodiments, the method provides a highly sensitive method of detecting and isolating SMDAs having a specific combination of multiple surface markers.

In embodiments, the methods described herein for detecting an SMDA are used in methods of isolating an SMDA. In embodiments, the kits described herein for use in detecting an SMDA can be used for isolating an SMDA.

In embodiments, the methods described herein for isolating an SMDA are used in detecting an SMDA. In embodiments, the constructs described herein for use in isolating an SMDA can be used for detecting an SMDA. In embodiments, the kits described herein for use in isolating an SMDA can be used for detecting an SMDA.

In embodiments, any method described herein for use in detecting an EV is used for detecting a SMDA. In embodiments, any method described herein for use in isolating an EV can be used for isolating a SMDA.

Multimarker isolation of EVs using stapling involves (i) immobilizing an SMDA of interest by binding it to a surface through a reversible linkage by, for example, binding a first surface protein on the SMDA to a capture entity immobilized on the surface by a cleavable linker, (ii) further binding or “stapling” an immobilized SMDA to the surface through one or more additional linkages that target one or more additional distinct features of the SMDA, typically through binding an oligonucleotide-conjugated entity to a second surface protein on the SMDA and connecting the oligonucleotide to a second oligonucleotide associated with the surface through e.g. hybridization or enzymatic ligation, and (iii) breaking the first linkage between the SMDAs and surface to release any SMDAs that were not also bound to the surface through the additional linkage(s) or “stapled” to the surface, thereby retaining only the captured SMDAs having the features targeted by the additional linkage(s).

The surface may be, but is not limited to, a particle, a bead, or a surface of a culture dish, culture well, or plate. The surface may be magnetic, or it may be coated with an electrode.

Such methods permit isolation and/or enrichment of specific populations of EVs or other SMDAs of interest with specific features e.g. EV with combinations of two or more surface proteins.

After capture of EVs, “stapling”, and removal of non-target EVs by breaking the first linkage (for example, via washing the surface in a stringent wash buffer to dissociated the strands of a duplex DNA linker, or through cutting a double stranded DNA linker using a sequence-specific endonuclease, or through a chemically cleavable linker), final release of the desired EV population is typically performed by breaking the attachment between the surface and additional linkages, e.g. by using a non-specific endonuclease such as DNase I to digest oligonucleotides where the additional linkages are provide by oligonucleotide-conjugated antibodies. This leaves antibodies bound to EVs through antibody:antigen interaction which can interfere with down downstream analysis of the selected population.

### Surface Marker Displaying Agents

SMDAs include naturally occurring, partially synthetic, or fully synthetic agents. In embodiments, the SMDA is a biologically relevant material or component. In general, a SMDA comprises a surface, typically a lipid bilayer, membrane, cell wall, or envelope, on which one or more markers are displayed.

In embodiments, the SMDA encapsulates components such as, e.g., proteins, nucleic acids, lipids, carbohydrates, small molecules such as hormones, cofactors, vitamins, minerals, salts, metals, metal-containing compounds, or combination thereof. Examples of SMDAs include cells (including prokaryotic cells, such as bacterial cells or archaeal cells; eukaryotic cells such as mammalian cells, insect cells, or plant cells); viruses and viral particles; cellular organelles such as nucleus, endoplasmic reticulum, Golgi apparatus, mitochondria, vacuoles, or chloroplast; vesicles such as lysosome, endosome, peroxisome, and liposome; and EVs or exosomes.

In embodiments, the methods provided herein enable capture of a SMDA of interest from a sample, wherein the SMDA of interest includes a unique co-localization of surface markers. In embodiments, certain markers can exclude unwanted populations of SMDAs (e.g., use of a mammalian cell-specific marker to exclude non-mammalian cells).

In embodiments, the SMDA is a cell, and the methods provided herein enable isolation and/or characterization of a cell of interest in a population of cells. In embodiments, the cell is a bacterial cell. In embodiments, the cell is an archaeal cell. In embodiments, the cell is a eukaryotic cell. In embodiments, the cell is a mammalian cell. In embodiments, the cell is an animal cell. In embodiments, the cell is a human cell. In embodiments, the cell is an insect cell. In embodiments, the cell is a plant cell. In embodiments, the cell is a yeast cell. In embodiments, the cell is a variant of a particular cell type. For example, the methods provided herein can be used to isolate abnormal cells, e.g., a cancer cell, from a sample of tissue or bodily fluid, for example, blood (e.g., comprising a mixture of cancer and non-cancer cells). In another example, the methods provided herein can be used to isolate a specific type of bacteria from a mixed bacterial sample, e.g., an environmental sample.

In embodiments, the methods provided herein enable identification of populations of cells in a sample. In embodiments, a large number of detection reagents for different surface markers can be screened in a single panel to determine all combinations of the surface markers present on the cells. In one example, a single panel can include antibodies to a selected number of CD markers, each antibody conjugated with a unique oligonucleotide as described herein. In embodiments, a single panel includes antibodies to all 350 CD markers. The unique oligonucleotides can be ligated upon formation of a complex between the cell of interest and the antibodies for the desired number of surface markers (e.g., three surface markers). The ligated oligonucleotides can then be sequenced to identify all cells having the three surface markers. Compared with conventional methods of cell isolation and identification using surface markers that rely upon a fluorescence or colorimetric output such as, e.g., flow cytometry, the present methods provide higher efficiency, e.g., by reducing processing and increasing throughput.

In embodiments, the SMDA is a virus or viral particle, and the methods provided herein enable isolation and/or characterization of a particular type of virus or viral particle. The present methods may facilitate the study of viruses or viral particles, as traditional methods (for example, flow cell-based methods) may not be able to accurately distinguish between different small viruses or viral particles.

In embodiments, the SMDA is a cellular organelle, and the methods provided herein enable isolation and/or characterization of an organelle of interest from a sample. Organelle isolation typically involves multiple rounds of subcellular fractionation and screening. The present methods may advantageously isolate an organelle of interest by using one or more surface markers unique to the organelle of interest. For example, TGN38 is a marker unique to the Golgi; VDAC1 is a marker unique to the mitochondria; cytochrome c reductase is a marker unique to the endoplasmic reticulum; and NUP98 is a marker unique to the nucleus.

In embodiments, the SMDA is a vesicle, and the methods provided herein enable isolation and/or characterization of a vesicle of interest from a sample. In embodiments, the vesicle is a lysosome, an endosome, or a peroxisome. Examples of lysosome-specific markers include, e.g., LAMP1, LC3, and ATG5. Examples of endosome-specific markers include, e.g., EEA1, Rab5, Rab7, and palladin. An example of a peroxisome-specific marker is catalase. In embodiments, the vesicle is a liposome. Liposomes can be artificial vesicles that include engineered surface markers.

In embodiments, the vesicle is an EV or an exosome. EVs are described herein.

In embodiments, the SMDA comprises a surface-associated marker. Unlike surface markers, surface-associated markers are generally not integrally expressed on the surface of a SMDA, but may be covalently or non-covalently bound to one or more surface markers and/or structural components of the surface. In embodiments, the surface-associated marker is associated with the membrane of a SMDA. In embodiments, the surface-associated marker is associated with a transmembrane protein of a SMDA. In embodiments, the surface-associated marker is a surface receptor. In embodiments, the SMDA is a cell. In embodiments, the SMDA is an EV.

### Extracellular Vesicles

EVs released from a variety of cells target recipient cells for intercellular communication and transfer a subset of genetic materials, proteins, lipids, and metabolites. EVs include a broad spectrum of vesicles secreted by several types of cells and the term is used as a collective one. These include exosomes, ectosomes, oncosomes, shed vesicles, microvesicles, and apoptotic bodies. Thus, EVs represent a broad spectrum of vesicles secreted by several types of cells. Major groups include exosomes (endosomal origin, 40-200 nm), microvesicles/ectosomes (plasma membrane origin, 100-1000 nm) and larger particles such as large-oncosomes (tumor cell origin, >1 um). The exact definition and nomenclature for each of these general vesicles classes has yet to be fully codified by the field due to their heterogeneous nature, herein, the term “EVs” is as defined by the International Society of Extracellular Vesicles (see Gardiner et al., Journal of Extracellular Vesicles 5(1):32945 (2016).

The isolation and assay methods provided herein enable capture of EVs of interest from the sample, wherein the EVs bear a unique co-localization of surface markers. In embodiments, certain markers can exclude certain unwanted populations of EVs (e.g., use of CD81 as detection marker to exclude platelet derived vesicles). In embodiments, some of the cell-type specific surface markers select EVs of particular origin (i.e. exosomes or ectosomes/microvesicles). In embodiments, the isolation methods exclude very large EVs, apoptotic bodies and cell debris from cell culture supernatants using common techniques like differential centrifugation, ultrafiltration and size-exclusion chromatography but do not otherwise distinguish between small EVs of various origin. In embodiments, the sample provided herein is initially depleted of undesirable EVs. In embodiments, plasma samples are depleted from platelet-derived EVs. In embodiments, anti-CD41 and CD61 beads are employed to deplete platelet-derived EVs.

While EVs secreted by neurons and various glial populations have been studied in vitro, isolating populations of EVs from biofluids remains elusive because no method of discriminating these cell-specific EVs has yet been developed. This invention provides methods of isolating populations based on the fact that combinations of surface markers define EVs secreted by specific cells such as CRC cells. The methods described herein thus take advantage of the fact that most proteins that are highly expressed on the surface of a particular cell line are also present on the surface of the EVs secreted in cultures of those cells.

In embodiments, the EV comprises a surface marker that is common to EVs. In embodiments, the first marker is common to EVs. In further embodiments, the marker common to EVs is a tetraspanin. Exemplary tetraspanins include CD9, CD37, CD63, CD 81, and CD82. In embodiments, a surface marker common to EVs is CD9, CD11a, CD18, CD26, CD29, CD35, CD45, CD46, CD47, CD48, CD50, CD51, CD55, CD63, CD71, CD73, CD81, CD82, CD95, CD104, CD151, CD276, or CD317.

In embodiments, the EV comprises a surface marker that is a surface adhesion protein. Exemplary surface adhesion proteins include, but are not limited to, EpCAM, E-Cadherin, N-cadherin, P-Cadherin, E-selectin, P-selectin, L1CAM, VE-cadherin, ITGB1, MCAM, ICAM-3, ITBG1, MCAM, ALCAM, NCAM1, Nectin-4, PECAM and ICAM-1. In embodiments, the EV comprises a surface marker that is a surface receptor. Exemplary surface receptors include, but are not limited to, EGFR, EphA2, TFRC, FasR, TNFR1, TNFR2, SCFR/Kit, FASR, IL-6R, FLT-1, MET, CXCR4, CXCR5, CCR2, EPCR, and VEGFR2. In embodiments, the EV comprises a surface marker that is an endothelial marker. Exemplary endothelial markers include, but are not limited to, CD146, PECAM, CD276, TEM7, TEM8, thrombomodulin, endoglin, PSGL-1, VE-cadherin, E-selectin, ICAM-1, and ICAM-3. In embodiments, the EV comprises a surface marker that is a tumor antigen. Exemplary tumor antigens include, but are not limited to, CEA, CA19.9, CA50, CA125, CA15.3, mesothelin, cytokeratin-8, E-cadherin, EGFR, EpCAM, EphA2, NCAM, P-cadherin, cMET, Flt-3L, TNFR-2, cKit, ErbB2, FAP-a, and ANXA1. In embodiments, the tumor antigen markers are pancreatic cancer markers. In embodiments, the tumor antigen markers are colorectal cancer markers. In embodiments, the EV comprises a surface marker that is a platelet EV marker. Exemplary platelet EV markers proteins include, but are not limited to, P-selectin, PECAM, CD63 and CD9.

In embodiments of the invention, at least one of EV surface markers is a central nervous system (CNS) cell marker. In additional embodiments, the EV surface marker is specific to a neuron, an astrocyte, an oligodendrocyte or a microglia. In embodiments, the EV surface marker is specific to a neuron. In embodiments, the EV surface marker specific to a neuron is L1CAM, NCAM, NRCAM, CHL1, Glu-R2, neurofascin, DAT1, CD90, CD24, N-cadherin, PSA-NCAM, synaptophysin, or combinations thereof. In embodiments, the neuron is a dopaminergic neuron, a GABAergic neuron, a cholinergic neuron, a serotonergic neuron or a glutamatergic neuron.

In embodiments, the EV surface marker is specific to an astrocyte. In embodiments, the surface marker specific to an astrocyte is ALDH1L1, GLT-1, GLAST, CD184, CD44, A2B5, aquaporin-4, ATP1B2 (ASCA-2), ceruloplasmin, CD80, CD86, or combinations thereof. In embodiments, the EV surface marker is specific to an oligodendrocyte. In embodiments, the surface marker specific to an oligodendrocyte is O4, PDGFRa, CSPG4, GD3, MOG, MBP, or combinations thereof. In embodiments, the EV surface marker is specific to a microglia. In embodiments, the microglia surface marker is Tmem119, CD11bF4/80, CD68, P2RY12, CXC3R1, or combinations thereof. In embodiments, the EV surface marker is a disease-specific biomarker.

In embodiments, the EV surface marker is specific to astrocytes and neurons. In embodiments, the surface marker specific to astrocytes and neurons is ALCAM CD166, CD40, FGFR3, GJA1 (connexin 43), integrin B1 (CD29), CD24, or combinations thereof. In embodiments, the surface marker specific to neurons is CD11, CD56, CD90, CD166, CD171, CD271, CD325, or combinations thereof. In embodiments, the surface marker specific to astrocytes is A2B5, ASCA, GJA1, GLAST-1, or combinations thereof.

In embodiments, the EV surface marker is specific to a T cell, a B cell, a dendritic cell, an NK cell, a monocyte, a macrophage, a granulocyte, a platelet, an erythrocyte, an endothelial cell, an epithelial cell, a stem cell precursor cell, a mesenchymal stem cell, a hematopoietic stem cell, a leukocyte, a T lymphocyte, or a B lymphocyte. T cells include, e.g., helper T cells, such as the subtypes Th1, Th2, Th9, Th17, Th22, and Tfh; regulatory T cells; killer T cells; γδ TCR+ T cells; and natural killer T cells.

In embodiments, the EV surface marker is specific to a T cell, a helper T cell, a regulatory T cell, a killer T cell, a γδ TCR+ T cell, or a natural killer T cell. In embodiments, the surface marker specific to a T cell is CD2, CD3, CD4, CD5, CD6, CD8, CD9, CD25, CD28, CD30, CD37, CD38, CD44, CD49b, CD52, CD53, CD56, CD57, CD62L, CD69, CD70, CD103, CD152, CD154, CD162, CD166, CD178, CD181, CD182, CD183, CD223, CD272, CD278, CD314, CD366, or combinations thereof. In embodiments, the surface marker specific to a helper T cell is CD5, CD6, CD45, CD62L, CD197(CCR7), a/b TCR, or combinations thereof. In embodiments, the surface marker specific to a helper T cell subtype Th1 is CD183(CXCR3), CD119 (IFNy Ra), CD195 (CCR5), CD218a(IL-18Ra), LT-BR, CD336 (TIM-3), or combinations thereof. In embodiments, the surface marker specific to a helper T cell subtype Th2 is CD194(CCR4), Crth2, CDwl98(CCR8), CRTH2, IL33-Ra, CD365(TIM-1), or combinations thereof. In embodiments, the surface marker specific to a helper T cell subtype Th17 is CD196(CCR6), CD161, or IL-23R. In embodiments, the surface marker specific to a helper T cell subtype Th22 is CCR10. In embodiments, the surface marker specific to a helper T cell subtype Tfh is CD185(CXCR5), CD84, CD126(IL-6Ra), CD150, CD154, CD252(OX40L), CD278(ICOS), CD279(PD1), or combinations thereof. In embodiments, the surface marker specific to a regulatory T cell is CD25, CD39, CD73, CD103, CD152(CTLA-4), GARP, GITR, or combinations thereof. In embodiments, the surface marker specific to a killer T cell is CD8. In embodiments, the surface marker specific to a γδ TCR+ T cell is γδ TCR. In embodiments, the surface marker specific to a natural killer T cell is CD56 (NCAM), CD11b, CD11c, CD16, CD32, CD49b, CD57, CD69, CD94, CD122, CD158, CD161 (NK1.1), CD244, CD314, CD319, CD328, CD355, Ly49, Ly108, Va24-J18 TCR, or combinations thereof.

In embodiments, the EV surface marker is specific to a B cell. In embodiments, the surface marker specific to the B cell is CD10, CD19, CD20, CD5, CD9, CDIIa, CD18, CD21, CD23, CD24, CD25, CD26, CD27, CD29, CD30, CD31, CD37, CD38, CD40, CD44, CD45, CD49b, CD49c, CD49d, CD50, CD52, CD53, CD54, CD57, CD58, CD62L, CD70, CD72 CD73, CD79a, CD80, CD95, CD102, CD119, CD120a, CD120b, CD124, CD138, CD166, CD223, CD267, CD269, CD319, or combinations thereof.

In embodiments, the EV surface marker is specific to a neutrophil. In embodiments, the surface marker specific to the neutrophil is CD11b, CD11c, CD15, CD16b, CD37, CD44, CD53, CD66b, CD87, CD114, CD116, CD162, CD172, CD181, CD182, or combinations thereof.

In embodiments, the EV surface marker is specific to a dendritic cell. In embodiments, the surface marker specific to the dendritic cell is CDIa, CD11c, CD23, CD33, CD40, CD45, CD49d, CD49e, CD52, CD53, CD58, CD73, CD80, CD83, CD115, CD120a, CD120b, CD123, CD201, CD207, CD208, CD209, CD223, CD271, or combinations thereof.

In embodiments, the EV surface marker is specific to a NK cell. In embodiments, the surface marker specific to the NK cell is CDIIa, CD11b, CD11c, CD16a, CD18, CD25, CD26, CD29, CD31, CD38, CD45, CD49b, CD49d, CD49e, CD50, CD53, CD56, CD57, CD58, CD59, CD62L, CD69, CD94, CD95, CD96, CD119, CD120a, CD120b, CD178, CD183, CD223, CD314, or combinations thereof.

In embodiments, the EV surface marker is specific to a monocyte or a macrophage. In embodiments, the surface marker specific to the monocyte or macrophage is CD4, CD9, CD11a, CD11b (integrin a-M), CD11c, CD13, CD14, CD15, CD16, CD16a, CD18, CD23, CD26, CD29, CD31, CD33, CD36, CD37, CD38, CD40, CD44, CD45, CD49a, CD49b, CD49c, CD49e, CD49f, CD50 (ICAM-3), CD51, CD52, CD53, CD54, CD57, CD58, CD59, CD61, CD62L, CD63, CD64, CD68, CD80, CD86, CD87, CD95, CD102, CD105, CD114, CD115, CD119, CD120a, CD120b, CD123, CD124, CD127, CD162, CD163, CD166, CD172, CD181, CD182, CD184, CD192 (CCR2), or combinations thereof.

In embodiments, the EV surface marker is specific to a granulocyte. In embodiments, the surface marker specific to the granulocyte is CD66b, CD4, CD9, CDIIa, CD13, CD14, CD15, CD18, CD29, CD31, CD33, CD44, CD45, CD50, CD58, CD59, CD63, CD95, CD119, CD120a, CD120b, CD123, CD178, or combinations thereof.

In embodiments, the EV surface marker is specific to a platelet. In embodiments, the surface marker specific to the platelet is CD23, CD9, CD29, CD31, CD36, CD41, CD44, CD49b, CD49f, CD51, CD61, CD62, CD63, CD102, CD107, CD120a, CD120b, CD140a, or combinations thereof.

In embodiments, the EV surface marker is specific to an erythrocyte. In embodiments, the surface marker specific to the erythrocyte is CD36, CD235a, CD49e, CD58, CD59, CD49e, CD58, CD235a, or combinations thereof.

In embodiments, the EV surface marker is specific to an endothelial cell. In embodiments, the surface marker specific to the endothelial cell is CD31, CD34, CD54, CD62E, CD90, CD105, CD106, CD141, CD144, CD146, CD162, CD181, CD182, CD01, CD309, PECAM, B7-H3, CD276, TEM7, TEM8, thrombomodulin, endoglin, PSGL-1, ICAM-1, ICAM-3, CD106 (VCAM-1), CD201 (EPCR), CD309 (VEGF-R2), CD40, ESAM, E-selectin, IL-1 R1, THSD1, VE-cadherin (CD144), VEGF-R1 (FLT-1), or combinations thereof.

In embodiments, the EV surface marker is specific to an epithelial cell. In embodiments, the surface marker specific to the epithelial cell is CD58, CD111, CD112, CD166, CD227, CD324, CD326, CD340, EpCAM, EGFR, EphA2, or E-cadherin. In embodiments, the surface marker specific to the endothelial or epithelial cell is CD9, CD10, CD13, CD26, CD29, CD31, CD34, CD49b, CD49c, CD49d, CD49e, CD49f, CD50, CD51, CD54, CD58, CD61, CD62E, CD62P, CD63, CD71, CD90, CD102, CD104, CD105, CD109, CD119, CD120a, CD120b, CD121a, CD123, CD124, CD133, CD140a, CD140b, CD144, CD146, CD166, CD178, or combinations thereof.

In embodiments, the EV surface marker is specific to a lymphoid cell. In embodiments, the surface marker specific to the lymphoid cell is CD3, CD4, CD8, or CD19. In embodiments, the EV surface marker is specific to a myeloid cell. In embodiments, the surface marker specific to the myeloid cell is CD15 or CD55b.

In embodiments, the EV surface marker is a cancer antigen. In embodiments, the cancer antigen is 5′-nucleotidase (CD73), B7-H3 (CD276), CA19.9, CA60, cadherin-1 (CD324), CD44v6, ADAM10 (CD156c), basigin (CD147), CD24, CD91, Cripto-1 (TSGF1), E-selectin (CD62e), FLT-3 ligand, AlCAM (CD166), Claudin-3, Claudin-4, EGFR, EGFRvIII, CDCP1 (CD318), CEACAM5 (CD66e), Ephrin receptor A2, FAP-a, Glypican-1, HIST2H2BE, HIST2H2BF, CD44, Galectin-3-binding-protein, MAGE3/6, Gamma-enolase (NSE), IL-2R, KIT (CD117), KNG2DL2 (ULBP-2), EpCAM (CD326), FasR (CD95), FasL, HER-2, ICAM-1 (CD54), Integrin A6 (CD49f), Integrin B4(CD104), Mucin-4, Prominin-1 (CD133), Wnt-2, Mucin-16, Mucin-18 (CD146), Sialyl Lewis X, Syndecan-1 (CD138), TNFR1 (CD120a), upaR (CD87), L1CAM (CD171), MET, MUC1 (CA15-3), RaphBlood group (CD151), Tspan8, EphB4, CEA, ALCAM (CD166), DCC (netrin 1 receptor), LRIG3, Nectin-4, TNFSF8, YES, Galectin-9, Vimentin, Cytokeratin, or combinations thereof.

In embodiments, the EV surface marker is specific to a leukocyte. In embodiments, the surface marker specific to a leukocyte is CD3, CD4, CD5, CD8, CD8A, CD10, CD11b, CD13, CD14, CD15, CD19, CD20, CD24, CD26, CD31, CD40, CD50, CD54, CD56, CD64, CD67, CD71, CD73, CD90, CD105, CD141, CD66b, CD162, CD166, or combinations thereof. In embodiments, the EV surface marker is specific to a tumor infiltrating leukocyte. In embodiments, the surface marker specific to a tumor infiltrating leukocyte is LAG-3, TIM-3, PD-1 (CD279), CD44, PD-L1, CTLA-4, CD28, or combinations thereof. In embodiments, the EV surface marker is an antigen presenting cell marker. In embodiments, the antigen presenting cell marker is CD80, CD86, CD83, or combinations thereof. In embodiments, the surface marker is an immuno-oncology marker. In embodiments, the immune-oncology marker is CD137, CD154, CD40, or combinations thereof.

In embodiments, the EV surface marker is specific to a stem cell. In embodiments, the surface marker specific to a stem cell is ABCG2 (CD338), CD9, CDiib, CD20, CD29, CD31, CD34, CD44, CD45, CD49f, CD56, CD73, CD81, CD90, CD95, CD105, CD117, CD118, CD133, CD144, CD146, CD166, CD184, DLK1, STRO-1, TNAP, CD24, SSEA-3, SSEA-4, TRA-1-60, TRA-1-81, or combinations thereof. In embodiments, the EV surface marker is specific to a mesenchymal stem cell. In embodiments, the surface marker specific to a mesenchymal stem cell is CD73, CD105, CD90, CD29 (ITGB1), CD44, CD166, CD13, CD14, CD10, CD146, CD24, CD271, DLK1, STRO-1, TNAP, or combinations thereof. In embodiments, the EV surface marker is specific to a hematopoietic stem cell. In embodiments, the surface marker specific to the hematopoietic stem cell is CD34, CD117, CD135, CD201, or combinations thereof.

In embodiments, the EV surface marker is a cell adhesion molecule, an integrin, a classical cadherin, a desmosomal cadherin, a protocadherin, an unconventional cadherin, a claudin, or a selectin. In embodiments, the cell adhesion molecule is EpCAM, E-cadherin, N-cadherin, P-cadherin, E-selectin, P-selectin, LiCAM, VE-cadherin, ITGB1, MCAM, ICAM1, ICAM2, ICAM3, ICAM4, ICAM5, VCAM1, PECAM-1, NCAM, or ALCAM. In embodiments, the integrin is VLA-1, VLA-2, VLA-3, VLA-4, VLA-5, VLA-6, LFA-1, MAC-1, CD 11c/CD18, CD41/CD61, virtonectin-R, or CD49d. In embodiments, the classical cadherin is CDH1, CHD2, CDH12, or CDH3. In embodiments, the desmosomal cadherin is DSG1, DSG2, DSG3, DSG4, DSC1, DSC2, or DSC3. In embodiments, the unconventional cadherin is CDH4, CDH5, CDH6, CDH7, CDH8, CDH9, CDH10, CDH11, CDH13, CDH15, CDH16, CDH17, CDH18, CDH19, CDH20, CDH21, CDH22, CDH23, CDH24, CDH26, CDH28. In embodiments, the claudin is CDLN1, CDLN2, CDLN3, CDLN4, CDLN5, CDLN6, CDLN7, CDLN8, CDLN9, CDLN10, CDLN11, CDLN12, CDLN13, CDLN14, CDLN15, CDLN16, CDLN17, CDLN18, CDLN19, CDLN20, CDLN21, CDLN22, CDLN23, CDLN24, or combinations thereof. In embodiments, the selectin is E-selectin, P-selectin, or L-selectin.

In embodiments, the EV surface marker is specific to a senescent cell. In embodiments, the surface marker specific to the senescent cell is DPP4, CD26, CD57, CD16, or combinations thereof.

In embodiments, the EV surface marker is specific to an adipose cell. In embodiments, the surface marker specific to the adipose cell is ALK7, CD300LG, GHR, GLUT4, TUSC5, or combinations thereof.

In embodiments, the EV surface marker is specific to a hepatocyte. In embodiments, the surface marker specific to the hepatocyte is ASGR1, ASGR2, ceruloplasmin (RAN-2), FATP5, hepatocyte specific antigen, LRP1 (A2MR), or combinations thereof.

In embodiments, the EV surface marker is specific to a myocyte. In embodiments, the surface marker specific to the myocyte is AdipoR2, a-sarcoglycan, d-sarcogylcan, ITGA7, M-cadherin (Cad 15), or combinations thereof.

In embodiments, the EV surface marker is specific to a cardiac cell such as a cardiomyocyte, fibroblast, endothelial cell, smooth muscle cell, or combination thereof. In embodiments, the surface marker specific to the cardiac cell is Connexin-43, N-Cadherin, ATP1A3, PKP2, Dystrophin, SIPRA, VCAM-1, CD77, Caveolin-3, Desmoglein-2, Angiotensin II type 1 receptors, EMILIN-2, POPDC2, KCNA6, Desmin, or combinations thereof. In embodiments, presence of the cardiac cell specific surface marker in a subject is associated with higher risk of cardiovascular disease.

In embodiments, the EV comprises a surface-associated marker. In embodiments, the surface-associated marker is covalently or non-covalently bound to one or more surface markers and/or structural components of the EV surface. In embodiments, the surface-associated marker is associated with the EV membrane. In embodiments, the surface-associated marker is associated with an EV transmembrane protein. In embodiments, the surface-associated marker is an immunomodulatory molecule. In embodiments, the surface-associated marker is a cytokine. In embodiments, the surface-associated marker is a surface receptor. In embodiments, the surface-associated marker is a costimulatory molecule, e.g., as described in Hodge et al., Front Biosci 11: 788-803 (2006) and Bugeon et al., Am JRespir Crit Care Med 162: S164-S168 (2000).

In embodiments, the EV comprises a surface marker or surface-associated marker specific to an infected cell, e.g., infected by a pathogen such as bacteria, fungi, or virus. In embodiments, the EV comprises a surface marker or surface-associated marker specific to an HIV-infected cell. In embodiments, the surface-associated marker is IL-2RA, IFN-GR1, TNFR1, TNFR2, IL-1R1, IL-1R2, IL-3R, IL-4Ra, IL-5Ra, IL-6Ra, IL-7Ra, IL-9R, IL-6Rb, Common β subunit, Common γ subunit, 4-1BB, CXCR1, CXCR2, CXCR3, CXCR4, CXCR5, CXCR6, CCR1, CCR2, CCR3, CCR4, CCR5, CCR6, CCR7, CCR8, CCR9, IL-10RA, IL-10RB2 IL-12RB, IL-13RA1, IL-13RA2, IL-15RA, IL-17RA, IL-17RC, IL-18R1, IL18RAP, TRAIL-R3, TACI, BAFF-R, BCMA, VEGF-R1, VEGF-R2, IL-21R, IL-22Ra1, IL-23R, IL-27R, IL-31RA, TGF-B1, TGF-B2, TGF-B3, G-CSFR, GM-CSFR, FasR, OX40, 4-1BB, CTLA-4, LAG3, B7-H3, ICOS, PD-1, TIM-3, TIGIT, GITR, CD27, CD28, BTLA, or combinations thereof. In embodiments, the surface-associated marker is IL-1RA, IL-1a, IL-1B, IL-2, IL-3, IL-4, IL-5, IL-6, IL-7, IL-8, IL-9, IL-10, IL-12p70, IL12/IL23p40, IL-13, IL-15, IL-16, IL-17A, IL-18, IL-21, IL-22, IL-23, IL-27, IL-29, IL-31, IL-33, IFN-γ, TNF-α, TNF-β, TSLP, Eotaxin, Eotaxin-3, IP-10, MCP-1, MCP-4, MCD, MIP-1a, MIP-1B, MIP-3a, TARC, VEGF-A, GM-CSF, G-CSF, TGF-B1, TGF-B2, TGF-B3, TALL-1, RANTES, CXCL1, TRAIL, APRIL, BAFF, LIGHT, PD-L1, PD-L2, OX40L, GITRL, 4-1BBL, or combinations thereof.

In embodiments, the EV comprises a viral envelope protein. In embodiments, the EV comprises an HIV-1 viral protein. In embodiments, the HIV-1 viral protein is surface protein gpi20 or transmembrane protein gp41. In embodiments, the EV comprises an HCV viral protein. In embodiments, the HCV viral protein is envelope glycoprotein E1 or envelope glycoprotein E2. In embodiments, the EV comprises an HSV-1 viral protein. In embodiments, the HSV-1 viral protein is envelope glycoprotein B (gB), envelope glycoprotein C (gC) or envelope glycoprotein D (gD). In embodiments, the EV comprises an HLTV-1 viral protein. In embodiments, the HIV-1 viral protein is surface protein gp46 or transmembrane protein gp21. In embodiments, the EV comprises an EBV viral protein. In embodiments, the EBV viral protein is membrane antigen gp350, envelope glycoprotein H (gH), envelope glycoprotein L (gL), or LMPi. In embodiments, the EV comprises a combination of viral envelope proteins and immune markers, e.g., about 5 to about 100 viral envelope proteins, about 5 to about 100 immune surface receptors, about 5 to about 100 immune receptor ligands, or combination thereof.

In embodiments, the EV comprises a surface marker specific to or associated with cancer, such as: CD12, ABCB5, ASCT2, CA19-9, CCR7, CD10, CD105 (Endoglin), CD11b (ITGAM), CD13 (Aminopeptidase N), CD133 (PROM1), CD137, CD14, CD146 (MCAM), CD151, CD16, CD163, CD166 (ALCAM), CD171 (L1CAM), CD18, CD206, CD227 (MUC1), CD24, CD25, CD271, CD29, CD326, CD34, CD38, CD40, CD44, CD49e, CD49f, CD54 (ICAM-1), CD56 (NCAM1), CD66b (CEACAM8), CD66e (CEA), CD68, CD70, CD71, CD80, CD86, CD90, CD95, CD98, CL12A, Claudin-18.2, CXCR4, DLL3, DLL4, EGFR, Ephrin-B2, ERBB2 (HER2), FRα, GLUT1, GPC3, GPNMB (Osteoactivin), CEACAM6, HLA-DR, IL-13Ra2, ITGB3, LAT1, LGR5, LIV-1, LRC15, MCT1, MCT4, Mesothelin, MET, MMP14, Mucin-16 (CA125), CA 15-3, N-cadherin (CD325), Nectin-4, Notchl, Neuropilin-1, P-Cadherin, PDGFR-α/β, PSMA, ROR1, ROR2, TEM8, Tie-2, TLR4, TNFR1, TRAIL-R1, TRAIL-R2, uPAR, VCAM-1, VEGFR-1, VEGFR-2, VEGFR-3, xCT, CA9, CA12, TMPRSS4, Claudin-3, FGFR1, E-Cadherin (CD324), RANKL, EPHA2, Notch3, KIT, FGFR2, INSR, IGF1R, PSCA, IGF1R, PDGFRB, CRIPTO, BRAF, CD19, FGFR2, CCR2, CCR4, CCR5, TNK2, DDR1, LOXL2, or combinations thereof. In embodiments, the EV comprises a surface marker specific to or associated with a breast cancer marker. In embodiments, the breast cancer marker is CD4, CD5, CD3, CD2, CD8A, or combinations thereof.

In embodiments, the EV comprises a surface marker specific to or associated with immune cells, tumor immune micro-environment, tumor stroma such as: VTCN1, BST2, CTLA4, MRC1, DPP4, CD274, CD276, PDCD1, PECAM1, CD33, ENTPD1, CD47, NT5E, CLEC12A, CXCL10, FAP, FASLG, LGALS9, IL13RA2, ITGB6, LAG3, ROR1, SIGLEC10, SIGLEC15, SIGLEC7, SIGLEC9, HAVCR2, TACSTD2, C10orf54, CD4, CD2, CSF1R, IL6R, CD28, CD22, KLRK1, CD79B, CCR4, ICOS, MARCO, SIRPA, or combinations thereof.

In embodiments, the EV comprises a surface marker specific to or associated with platelets such as: PECAM1, CD36, CD40LG, ITGA2B, GP1BA, ITGB3, SELP, CD63, CLEC2A, GP6, TREM1, CD42a, CD42b, CD41, CD61, or combinations thereof.

In embodiments, the EV is an exosome, a micro-vesicle or a large-oncosome.

### Proximity Ligase-Ligase (Two Ligation)

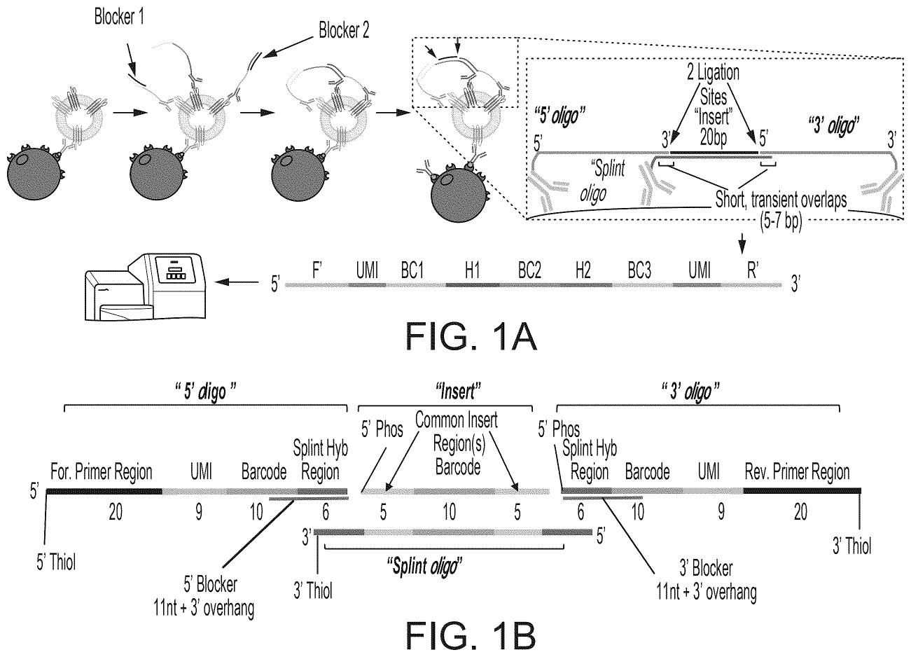

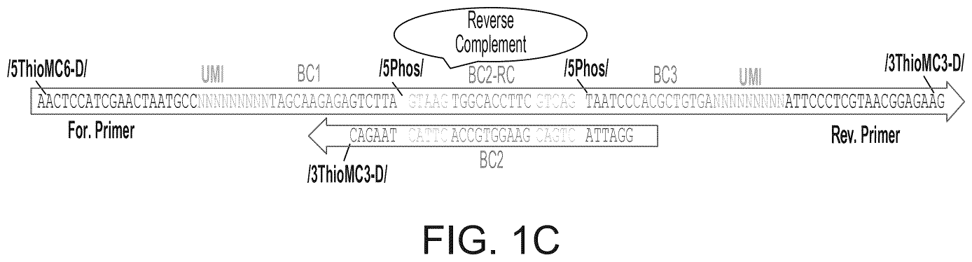

The present disclosure provides an assay format using two-site ligation (proximity ligation; PLL), embodiments of which are shown in FIGS. 1A-C. In embodiments, this reaction is at least about 50-fold to about 100-fold more efficient than the PEL reaction at converting oligonucleotides on antibody triplets into full-length product. In embodiments, the PLL reaction is at least about 50-fold more efficient than the PEL reaction. In embodiments, the PLL reaction is at least about 100-fold more efficient than the PEL reaction. It also allows for reduced length of the hybridization regions from 10 bases to as low as 5 such that these are only transient interactions. The PLL reaction shows a reduction in non-specific background over PEL, which is important as the number of antibodies in the pool is scaled to much higher numbers. Overall, it is a simpler, more efficient, more specific system than PEL. This change in the assay may also enable homogenous assays.

In embodiments, provided herein is a method of determining surface markers of a SMDA comprising contacting the SMDA with: (i) a capture reagent bound to a surface, (ii) a plurality of binding reagents, and (iii) an oligonucleotide insert. In embodiments, each binding reagent comprises a detection sequence comprising a unique barcode oligonucleotide sequence. In embodiments, when at least three binding reagents bind to three surface markers of the SMDA, an output oligonucleotide is generated that comprises the unique barcode oligonucleotide sequences of each of the three binding reagents. In embodiments, the output oligonucleotide is capable of being sequenced to identify the three surface markers of the SMDA. In embodiments, the output oligonucleotide is single-stranded. In embodiments, the output oligonucleotide is double-stranded.

In embodiments, the plurality of binding reagents comprises: a) a first binding reagent comprising a first detection sequence that comprises (i) a first hybridization sequence, wherein at least a portion of the first hybridization sequence is hybridized or complexed with a first blocker oligonucleotide, (ii) a first primer site, and (iii) a first connecting sequence hybridized to a complementary sequence on the first binding reagent; b) a second binding reagent comprising a second detection sequence that comprises (i) a second hybridization sequence, (ii) a third hybridization sequence, (iii) a fourth hybridization sequence, and (iv) a second connecting sequence hybridized to a complementary sequence on the second binding reagent; and c) a third binding reagent comprising a third detection sequence that comprises (i) a fifth hybridization sequence, wherein at least a portion of the fifth hybridization sequence is hybridized or complexed with a second blocker oligonucleotide, (ii) a second primer site, and (iii) a third connecting sequence hybridized to a complementary sequence on the third binding reagent.

FIG. 1A shows an embodiment of a construct that can be used in a PLL assay on intact EVs using a pool of three libraries of antibody conjugates, and a close up schematic of the hybridization of the oligonucleotides in an embodiment of the PLL assay. F′ and R′ refer to forward and reverse primer sequences, respectively. In some embodiments, a forward sequence comprises a 3′ to 5′ orientation or directionality. In embodiments, a reverse sequence comprises a 3′ to 5′ orientation or directionality. In some embodiments, a forward sequence comprises a 5′ to 3′ orientation or directionality. In embodiments, a reverse sequence comprises a 5′ to 3′ orientation or directionality. BC refers to barcode sequences and H1 and H2 represent the sequences formed by the overlap of the two common overlap sequences. FIG. 1B illustrates the details on the oligonucleotide construct and blockers used in FIG. 1A. FIG. 1C shows a library preparation illustration of the amplicon sequence of the final extension product produced when all three EV surface markers are present and includes the sequences corresponding to those from the oligonucleotides on the 5′-, splint and 3′-oligonucleotide-conjugated antibodies shown in FIG. 1A.

In embodiments, the first hybridization sequence and the second hybridization sequence are complementary. In embodiments, the fourth hybridization sequence and the fifth hybridization sequence are complementary. In embodiments, the third hybridization sequence is complementary to the oligonucleotide insert sequence. In embodiments, the third detection sequence is releasably bound to the surface. In embodiments, the first connecting sequence is hybridized to a complementary sequence conjugated to the first binding reagent. In embodiments, the second connecting sequence is hybridized to a complementary sequence conjugated to the second binding reagent. In embodiments, the third connecting sequence is hybridized to a complementary sequence conjugated to the third binding reagent. In embodiments, the first, second, and third connecting sequences are unique. In embodiments, the first, second, and third connecting sequences are identical.

In embodiments, generating the output oligonucleotide comprises ligating the hybridized first detection sequence to the hybridized oligonucleotide insert, and ligating the hybridized oligonucleotide insert to the third detection sequence to form a single-strand output oligonucleotide.

In embodiments, the method employs multiple different capture reagents and multiple different first, second, and third binding reagents to allow combinatorial sequencing and analysis of markers.

In embodiments, the capture reagent is attached to the surface. In embodiments, the capture reagent is releasably attached to the surface. In embodiments, the capture reagent is non-releasably attached to the surface. In embodiments, the capture reagent comprises a restriction site. In embodiments, the capture reagent comprises a Uracil-DNA Glycosylase 1 (UDG1) linkage. UDG is a DNA repair enzyme that removes uracil from DNA. In embodiments, the capture reagent is attached to the surface through a binding interaction comprising antibody or antigen binding fragment thereof/antigen or epitope or hapten or mimotope, antigen/antibody or antigen binding fragment thereof, ligand/receptor, receptor/ligand, oligonucleotide/oligonucleotide, hapten/antibody or antigen binding fragment thereof, epitope/antibody or antigen binding fragment thereof, mimitope/antibody or antigen binding fragment thereof, or aptamer/target molecule. In embodiments, the capture reagent is bound to the surface through a streptavidin/biotin or avidin/biotin binding interaction.

In embodiments of the methods of the disclosure, the capture reagent is releasably bound to the surface. In embodiments, the capture reagent is releasably bound to the surface by a labile linker. In embodiments, the labile linker is a heat-labile, a photolabile, or a chemically labile linker. In additional embodiments, the labile linker is an oligonucleotide that is complementary to an oligonucleotide bound to the surface or is an oligonucleotide comprising a restriction site cleavable by a restriction endonuclease. In embodiments, the labile linker is a small molecule that binds to a protein on the surface. In embodiments, the capture reagent is biotinylated, and the surface is coated with streptavidin. The surface can be, for example, a Meso Scale Discovery (MSD) plate electrode or a particle. In some embodiments, the surface is directly coated with the capture reagent. In embodiments, releasing the capture agent from the surface comprises denaturing the labile linker.

In embodiments of the method of detecting, the capture reagent binds to a surface marker common to EVs. Surface markers common to EVs are described herein. In embodiments, the marker is a tetraspanin. In embodiments, the tetraspanin is CD9, CD63, or CD81. In embodiments, the capture reagent binds to a surface marker that is not common to EVs. In embodiments, the capture reagent binds to a surface marker selected from CD2, CD3, CD4, CD5, CD8, CD10 (NEP), CD11b (ITGAM), CD13(AAP), CD14, CD15 (SSEA-1), CD16 (FcγRIII), CD18 (ITGB2), CD25(IL-2Ra), CD26(DPPIV), CD28, CD29 (ITGB1), CD31 (PECAM-1), CD32b (FcγRII), CD33 (Siglec-3), CD36 (GPIV), CD38, CD40, CD41 (GP2B), CD42b(GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM1), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166(ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1(VEGFR1) and GLUT1 (SLC2A1).

In embodiments, each of the binding reagents comprises an antibody or antigen binding fragment thereof, antigen, ligand, receptor, oligonucleotide, hapten, epitope, mimitope, lipid binding protein, carbohydrate binding protein, DNA aptamer or RNA aptamer. Thus, in embodiments, the binding reagent/surface marker pairs comprise antibody or antigen binding fragment thereof/antigen or epitope or hapten or mimotope, antigen/antibody or antigen binding fragment thereof, ligand/receptor, receptor/ligand, oligonucleotide/oligonucleotide, hapten/antibody or antigen binding fragment thereof, epitope/antibody or antigen binding fragment thereof, mimitope/antibody or antigen binding fragment thereof, lipid binding protein/target lipid, carbohydrate binding protein/target carbohydrate, or aptamer/target molecule.

In embodiments, each of the detection sequences, blocker oligonucleotides, and/or connecting sequences comprise DNA, RNA, LNA bases or any combination thereof. In embodiments, the bases are modified. In embodiments, the modified bases comprise, for e.g., 2′-O-methoxy-ethyl Bases (2′-MOE) (e.g., 2-MethoxyEthoxy A, 2-MethoxyEthoxy MeC, 2-MethoxyEthoxy G, and/or 2-MethoxyEthoxy T); 2′-O-Methyl RNA Bases (e.g., 2′-O-Methyl RNA Bases); Fluoro Bases (e.g., Fluoro C, Fluoro U, Fluoro A, and/or Fluoro G); 2-Aminopurine, 5-Bromo dU, deoxyUridine, 2,6-Diaminopurine (2-Amino-dA), Dideoxy-C, deoxyInosine, Hydroxymethyl dC, inverted dT, Iso-dG, Iso-dC, inverted Dideoxy-T, 3′-3′-inverted thymine, 5-Methyl dC, 5-Nitroindole, Super T (5-hydroxybutynl-2′-deoxyuridine), Super G (8-aza-7-deazaguanosine) and/or combinations thereof.

In embodiments of the method, the third binding reagent binds to a third surface marker on the EV. In embodiments, the third binding reagent is attached to the same surface as the capture reagent.

In embodiments of the method, each unique binding reagent comprises a detection sequence comprising a unique barcode oligonucleotide sequence. In embodiments, when at least three unique binding reagents bind to three unique surface markers of the SMDA, an output oligonucleotide is generated that comprises the barcode oligonucleotide sequences of each of the three unique binding reagents. In embodiments, the output oligonucleotide is capable of being sequenced to identify the three unique surface markers of the SMDA.

In embodiments of the method, the plurality of binding reagents comprises: a. a first binding reagent comprising a first detection sequence that comprises a first hybridization sequence, and a first primer site; b. a second binding reagent comprising a second detection sequence that comprises a second hybridization sequence, a third hybridization sequence, and a fourth hybridization sequence; and c. a third binding reagent comprising a third detection sequence that comprises a fifth hybridization sequence, and a second primer site.

In embodiments of the method, the first hybridization sequence and the second hybridization sequence are complementary. In embodiments, the fourth hybridization sequence and the fifth hybridization sequence are complementary. In embodiments, the third hybridization sequence is complementary to the oligonucleotide insert sequence. In embodiments, generating the single output oligonucleotide comprises ligating the hybridized first detection sequence to the hybridized oligonucleotide insert, and ligating the hybridized oligonucleotide insert to the third detection sequence.

In embodiments, the first binding reagent may also be referred to herein as a “5-prime conjugate,” or “5′ conjugate”, the second binding reagent may also be referred to as a “splint conjugate,” and the third binding reagent may also be referred to as a “3-prime conjugate” or “3′ conjugate”. In embodiments, the term “conjugate” refers to an oligonucleotide attached to a binding reagent. In embodiments, the term “conjugate” refers to a reagent comprising both an antibody or antigen-binding fragment, and an oligonucleotide. In embodiments, the term “attached” refers to covalently bonded or non-covalently bonded, either directly or indirectly, e.g., through a linker. In embodiments the term “oligonucleotide” refers to a sequence of RNA, LNA, or DNA nucleotides, or any combination thereof, and can be single or double stranded. The terms “oligonucleotide” and “sequence” may be used interchangeably throughout the application and, unless stated otherwise, refer to the same thing. For example, a “detection oligonucleotide” or “detection sequence” have the same meaning.

In embodiments of the method, the first detection oligonucleotide may also be referred to herein as a “5-prime sequence”, or “5-prime oligonucleotide”, or the second detection oligonucleotide” may also be referred to as a “splint sequence” or “splint oligonucleotide” and the third detection oligonucleotide may also be referred to as a “3-prime sequence” or “3-prime oligonucleotide.” In embodiments, the first binding reagent plays a role similar to a “capture reagent” as described in other methods herein, and any characteristic of a capture reagent provided herein may also apply to a first binding reagent. In embodiments, the first detection sequence may also be referred to herein as a “5′ conjugate oligonucleotide”, the second detection sequence may also be referred to herein as a “splint conjugate oligonucleotide”, and the third detection sequence may also be referred to herein as a “3′ conjugate oligonucleotide”.

In some embodiments of the method, the capture reagent binds to a surface marker common to EVs. In some embodiments of the method, the first binding reagent binds to a surface marker common to EVs. Surface markers common to EVs are described herein. In embodiments, the marker is a tetraspanin. In embodiments, the tetraspanin is CD9, CD63, or CD81. In embodiments, the first binding reagent or capture reagent binds to a surface marker that is not common to EVs. In embodiments, the first binding reagent or capture reagent binds to a surface marker selected from CD2, CD3, CD4, CD5, CD8, CD10 (NEP), CD11b (ITGAM), CD13(AAP), CD14, CD15 (SSEA-1), CD16 (FcγRIII), CD18 (ITGB2), CD25(IL-2Ra), CD26(DPPIV), CD28, CD29 (ITGB1), CD31 (PECAM-1), CD32b (FcγRII), CD33 (Siglec-3), CD36 (GPIV), CD38, CD40, CD41 (GP2B), CD42b(GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM11), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166(ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1(VEGFR1) and GLUT1 (SLC2A1).

In embodiments of the methods herein, each of the first, second and third binding reagents comprises an antibody or antigen binding fragment thereof, antigen, ligand, receptor, oligonucleotide, hapten, epitope, mimitope, lipid binding protein, carbohydrate binding protein, DNA aptamer or RNA aptamer.

The hybridization sequences are complementary sequences between the different oligonucleotide sequences in the constructs. These hybridization sequences may also be referred to as “common overlap” or “overlap” sequences. In embodiments of the method, each hybridization sequence has a length of about 5-10 nucleotides. In embodiments, each hybridization sequence has a length of about 5-7 nucleotides. In embodiments, each hybridization sequence has a length of about 3-15 nucleotides. In embodiments, each hybridization sequence has a length of about 4-13 nucleotides. In embodiments, each hybridization sequence has a length of about 5-12 nucleotides. In embodiments, each hybridization sequence has a length of about 5-11 nucleotides. In embodiments, each hybridization sequence has a length of about 3, 4, 5, 6, 7, 8, 9, 10, 11, 12, 13, 14 or 15 nucleotides. In embodiments, each hybridization sequence is the same length. In embodiments, one or more of the hybridization sequences is a different length. In embodiments, the hybridization includes one or more non-canonical base-pairing.

In embodiments, the hybridization sequences are complexed to blocker oligonucleotides. In embodiments of the method, each blocker oligonucleotide has a length of about 5-20 nucleotides. In embodiments, each blocker oligonucleotide has a length of about 5-15 nucleotides. In embodiments, each blocker oligonucleotide has a length of about 5-10 nucleotides. In embodiments, each blocker oligonucleotide has a length of about 4-13 nucleotides. In embodiments, each blocker oligonucleotide has a length of about 5, 6, 7, 8, 9, 10, 11, 12, 13, 14 or 15 nucleotides. In embodiments, each blocker oligonucleotide is the same length. In embodiments, one or more of the blocker oligonucleotides is a different length.

In embodiments, any of the first, second or third detection sequences can comprise one or more additional barcode sequences. In embodiments, the first detection sequence further comprises a second barcode sequence between the first hybridization sequence and the first primer site. In embodiments, the second barcode sequence of the first detection sequence is a unique molecular identifier. In embodiments, the third detection sequence further comprises a second barcode sequence between the fifth hybridization sequence and the second primer site. In embodiments, the second barcode sequence of the third detection sequence is a unique molecular identifier.

A “barcode sequence” or “barcode oligonucleotide sequence,” as used herein, refers to a short nucleotide (typically between about 5 and about 40 nucleotides in length) that allows a corresponding nucleotide or molecule to be identified. In embodiments, the corresponding nucleotide or molecule is attached to the barcode sequence. In embodiments, the molecule is a peptide, a protein, a protein complex, an antibody, or a vesicle. In embodiments, the barcode sequence is a unique nucleotide identifiable by sequencing. In embodiments, the barcode sequence is hybridizable to a complementary detectable probe. In such embodiments, the complementary detectable probe hybridizes to the barcode sequence, allowing the corresponding nucleotide or molecule to be detected. Barcode technologies are described in, e.g., Winzeler et al., Science 285:901-906 (1999), Eason et al., Proc Natl Acad Sci 101(30):11046-11051 (2004), and Fredriksson et al., Nature Methods 4(4):327-329 (2007), each of which is herein incorporated by reference in its entirety.

In embodiments of the methods herein, Unique molecular identifiers (UMIs) are used as the barcode. UMIs are a type of molecular barcoding that can provide error correction and increased accuracy during sequencing. These molecular barcodes are short sequences used to uniquely tag each molecule in a sample library. UMIs are used for a wide range of sequencing applications, many around PCR duplicates in DNA and cDNA. UMI deduplication is also useful for RNA-seq gene expression analysis and other quantitative sequencing methods. Typically, the length of UMIs is chosen so that the number of possible UMI codes is greater than the number of molecules in the library. In embodiments the length is chosen so that the number of possible UMI codes is much larger than the number of molecules, ensuring that essentially no molecules in the library receive the same UMI code. In embodiments the length is chosen so that the number of possible UMI codes is much larger than the number of sequencing reads to ensure that essentially no sequencing reads have the same UMI codes. In embodiments, the length is chosen so that the number of possible UMI codes is less than the number of sequencing reads to ensure that each molecule is sequenced multiple times. This approach can be used to distinguish rare variants from sequencing errors or synthesis errors. In embodiments, the UMI is used to determine the molecular diversity in a library by counting UMI repeats.

In embodiments the UMI is generated by the inclusion of mixed or degenerate bases during the oligonucleotide synthesis process. In embodiments the UMI is split into 2 or more regions separated by one or more nucleotides that are not part of the UMI. In embodiments, the UMI has a length of about 10-20 nt. In embodiments, the UMI has a length of about 12-18 nt. In embodiments, the UMI has a length of about 15-17 nt.

In embodiments of the method, each barcode has a length of about 5-10 nucleotides. In embodiments, each barcode has a length of about 5-7 nucleotides. In embodiments, each barcode has a length of about 3-15 nucleotides. In embodiments, each barcode has a length of about 4-13 nucleotides. In embodiments, each barcode has a length of about 5-12 nucleotides. In embodiments, each barcode has a length of about 5-11 nucleotides. In embodiments, each barcode has a length of about 3, 4, 5, 6, 7, 8, 9, 10, 11, 12, 13, 14 or 15 nucleotides.

In embodiments, the first detection sequence and third detection sequence have a length of from about 35 to about 55 nucleotides. In embodiments, the first detection sequence and third detection sequence have a length of from about 40 to about 50 nucleotides. In embodiments, the first detection sequence and third detection sequence have a length of from about 30 to about 55 nucleotides. In embodiments, the first detection sequence and third detection sequence have a length of from about 50 to about 100 nucleotides. In embodiments, the first detection sequence has a length of from about 50 to about 100 nucleotides. In embodiments, the first detection sequence has a length of about 64 nucleotides. In embodiments, the first detection sequence has a length of from about 20 to about 60 nucleotides. In embodiments, the first detection sequence has a length of about 41 nucleotides. In embodiments, the third detection sequence has a length of from about 50 to about 100 nucleotides. In embodiments, the third detection sequence has a length of about 87 nucleotides. In embodiments, the third detection sequence has a length of from about 30 to about 70 nucleotides. In embodiments, the first detection sequence has a length of about 57 nucleotides. In embodiments, the first detection sequence and third detection sequence have a length of from about 20 to about 60 nucleotides. In embodiments, the first detection sequence and third detection sequence have a length of about 35, 36, 37, 38, 39, 40, 41, 42, 43, 44, 45, 46, 47, 48, 49, 50, 51, 52, 53, 54 or 55 nucleotides. In embodiments, the first detection sequence and third detection sequence are the same length. In embodiments, the first detection sequence and third detection sequence are different lengths. In some embodiments, the first detection sequence comprises at least 20%, at least 30%, at least 40%, at least 50%, at least 60%, at least 70%, at least 80%, at least 90%, at least 95%, at least 99%, or 100% sequence identity to SEQ ID NO: 9. In some embodiments, the third detection oligonucleotide comprises at least 20%, at least 30%, at least 40%, at least 50%, at least 60%, at least 70%, at least 80%, at least 90%, at least 95%, at least 99%, or 100% sequence identity to SEQ ID NO: 12.

In embodiments, the second detection sequence has a length of from about 18 to about 38 nucleotides. In embodiments, the second detection sequence has a length of from about 23 to about 33 nucleotides. In embodiments, the second detection sequence has a length of from about 20 to about 32 nucleotides. In embodiments, the second detection sequence has a length of from about 22 to about 30 nucleotides. In embodiments, the second detection sequence has a length of about 18, 19, 20, 21, 22, 23, 24, 25, 26, 27, 28, 29, 30, 31, 32, 33, 34, 35, 36, 37 or 38 nucleotides. In some embodiments, the second detection sequence comprises at least 20%, at least 30%, at least 40%, at least 50%, at least 60%, at least 70%, at least 80%, at least 90%, at least 95%, at least 99%, or 100% sequence identity to SEQ ID NO: 7 or 10.

In embodiments, the oligonucleotide insert has a length that allows it to hybridize with the complete sequence of the second detection sequence (splint oligonucleotide) that is not hybridized by the first or third detection sequences. In embodiments, the oligonucleotide insert has a length of about 10 to about 30 nucleotides. In embodiments, the oligonucleotide insert has a length of about 15 to about 25 nucleotides. In embodiments, the oligonucleotide insert has a length of about 20 nucleotides. In some embodiments, the oligonucleotide insert comprises at least 20%, at least 30%, at least 40%, at least 50%, at least 60%, at least 70%, at least 80%, at least 90%, at least 95%, at least 99%, or 100% sequence identity to SEQ ID NO: 11.

In embodiments of the method comprising three binding reagents and a capture reagent, the first binding reagent comprises a first conjugating oligonucleotide for “tethering” or “anchoring” the first detection sequence. In embodiments, the first detection sequence comprises a first connecting sequence, a first primer site, a first barcode sequence, a first blocker complement sequence, and a first hybridization sequence. In embodiments, the first connecting sequence is complementary to at least a portion of the first conjugating oligonucleotide. In some embodiments, the first connecting sequence comprises the first primer site. In embodiments, the first primer site is complementary to a forward primer. In some embodiments, the first conjugating oligonucleotide is linked directly or indirectly to a biotin. In some embodiments, the first conjugating oligonucleotide is linked directly or indirectly to an antibody. In some embodiments, the term “conjugating oligonucleotide” may be used interchangeably with the terms “tethering oligonucleotide.”

In some embodiments, the first conjugating oligonucleotide is about 10 to about 70 nucleotides long. In some embodiments, the first conjugating oligonucleotide is about 15 to about 30 nucleotides long. In some embodiments, the first conjugating oligonucleotide is about 5 to about 25 nucleotides long. In some embodiments, the first conjugating oligonucleotide is about 5, 10, 15, 20, 25, 30, 35, 40, 45, 50, 55, 60, 65, or 70 nucleotides long. In some embodiments, the first conjugating oligonucleotide is about 40 nucleotides long. In some embodiments, the first conjugating oligonucleotide comprises at least 20%, at least 30%, at least 40%, at least 50%, at least 60%, at least 70%, at least 80%, at least 90%, at least 95%, at least 99%, or 100% sequence identity to SEQ ID NO: 8.

In some embodiments, the first connecting sequence is about 10 to about 40 nucleotides long. In some embodiments, the first connecting sequence is about 15 to about 30 nucleotides long. In some embodiments, the first connecting sequence is about 5 to about 25 nucleotides long. In some embodiments, the first connecting sequence is about 5, 10, 15, 20, 25, 30, 35, 40, 45, or 50 nucleotides long. In some embodiments, the first connecting sequence is about 20 nucleotides long.

In some embodiments, the first primer site is about 5 to about 40 nucleotides long. In some embodiments, the first primer site is about 15 to about 30 nucleotides long. In some embodiments, the first primer site is about 5 to about 25 nucleotides long. In some embodiments, the first primer site is about 5, 10, 15, 20, 25, 30, 35, 40, 45, or 50 nucleotides long. In some embodiments, the first primer site is about 20 nucleotides long. In some embodiments, the first primer site comprises at least 20%, at least 30%, at least 40%, at least 50%, at least 60%, at least 70%, at least 80%, at least 90%, at least 95%, at least 99%, or 100% sequence identity to SEQ ID NO: 1.

In some embodiments, the second binding reagent of the method disclosed herein comprises a second conjugating oligonucleotide for “tethering” or “anchoring” the second detection sequence. In embodiments, the second detection sequence comprises a second connecting sequence, a second hybridization sequence, a third hybridization sequence comprising a second barcode sequence, and a fourth hybridization sequence. In embodiments, the second connecting sequence is complementary to at least a portion of the second conjugating oligonucleotide. In some embodiments, the second conjugating oligonucleotide comprises a first spacer region. In some embodiments, the second conjugating oligonucleotide is linked directly or indirectly to an antibody.

In some embodiments, the second conjugating oligonucleotide is about 10 to about 70 nucleotides long. In some embodiments, the second conjugating oligonucleotide is about 15 to about 30 nucleotides long. In some embodiments, the second conjugating oligonucleotide is about 5 to about 25 nucleotides long. In some embodiments, the second conjugating oligonucleotide is about 5, 10, 15, 20, 25, 30, 35, 40, 45, 50, 55, 60, 65, or 70 nucleotides long. In some embodiments, the second conjugating oligonucleotide is about 20 nucleotides long. In some embodiments, the second conjugating oligonucleotide is about 40 nucleotides long. In some embodiments, the second conjugating oligonucleotide comprises at least 20%, at least 30%, at least 40%, at least 50%, at least 60%, at least 70%, at least 80%, at least 90%, at least 95%, at least 99%, or 100% sequence identity to SEQ ID NO: 14.

In some embodiments, the second connecting sequence is about 10 to about 40 nucleotides long. In some embodiments, the second connecting sequence is about 15 to about 30 nucleotides long. In some embodiments, the second connecting sequence is about 5 to about 25 nucleotides long. In some embodiments, the second connecting sequence is about 5, 10, 15, 20, 25, 30, 35, 40, 45, or 50 nucleotides long. In some embodiments, the second connecting sequence is about 20 nucleotides long.

In some embodiments, the first spacer region is about 10 to about 40 nucleotides long. In some embodiments, the first spacer region is about 15 to about 30 nucleotides long. In some embodiments, the first spacer region is about 5 to about 25 nucleotides long. In some embodiments, the first spacer region is about 5, 10, 15, 20, 25, 30, 35, 40, 45, or 50 nucleotides long. In some embodiments, the first spacer region is about 20 nucleotides long.

In embodiments, the third binding reagent of the method disclosed herein comprises a third conjugating oligonucleotide for “tethering” or “anchoring” the third detection sequence. In embodiments, the third detection sequence comprises a fifth hybridization sequence, a second blocker complement sequence, a third barcode sequence, a second primer site, a third connecting sequence, and a third primer site. In some embodiments, the third detection sequence comprises a second spacer region. In embodiments, the third connecting sequence is complementary to at least a portion of the third conjugating oligonucleotide. In some embodiments, the third connecting sequence comprises the second primer site. In embodiments, the second primer site is complementary to a reverse primer. In embodiments, the third primer site is complementary to a strand-displacement primer. In some embodiments, the third conjugating oligonucleotide comprises a third spacer region. In some embodiments, the third conjugating oligonucleotide is linked directly or indirectly to an antibody.

In some embodiments, the third conjugating oligonucleotide is about 10 to about 70 nucleotides long. In some embodiments, the third conjugating oligonucleotide is about 15 to about 30 nucleotides long. In some embodiments, the third conjugating oligonucleotide is about 5 to about 25 nucleotides long. In some embodiments, the third conjugating oligonucleotide is about 5, 10, 15, 20, 25, 30, 35, 40, 45, 50, 55, 60, 65, or 70 nucleotides long. In some embodiments, the third conjugating oligonucleotide is about 40 nucleotides long. In some embodiments, the third conjugating oligonucleotide is about 20 nucleotides long. In some embodiments, the third conjugating oligonucleotide comprises at at least 20%, at least 30%, at least 40%, at least 50%, at least 60%, at least 70%, least 80%, at least 90%, at least 95%, at least 99%, or 100% sequence identity to SEQ ID NO: 14. In some embodiments, the third conjugating oligonucleotide and the second conjugating oligonucleotide are the same. In some embodiments, the third conjugating oligonucleotide and the second conjugating oligonucleotide are distinct.

In some embodiments, the third connecting sequence is about 10 to about 40 nucleotides long. In some embodiments, the third connecting sequence is about 15 to about 30 nucleotides long. In some embodiments, the third connecting sequence is about 5 to about 25 nucleotides long. In some embodiments, the third connecting sequence is about 5, 10, 15, 20, 25, 30, 35, 40, 45, or 50 nucleotides long. In some embodiments, the third connecting sequence is about 20 nucleotides long.

In some embodiments, the second spacer region is about 1 to about 40 nucleotides long. In some embodiments, the second spacer region is about 1 to about 10 nucleotides long. In some embodiments, the second spacer region is about 3 to about 15 nucleotides long. In some embodiments, the second spacer region is about 1, 2, 3, 4, 5, 6, 7, 8, 9, or 10 nucleotides long. In some embodiments, the second spacer region is about 3 nucleotides long.

In some embodiments, the third spacer region is about 10 to about 40 nucleotides long. In some embodiments, the third spacer region is about 15 to about 30 nucleotides long. In some embodiments, the third spacer region is about 5 to about 25 nucleotides long. In some embodiments, the third spacer region is about 5, 10, 15, 20, 25, 30, 35, 40, 45, or 50 nucleotides long. In some embodiments, the third spacer region is about 20 nucleotides long.

In some embodiments, the second primer site is about 5 to about 40 nucleotides long. In some embodiments, the second primer site is about 15 to about 30 nucleotides long. In some embodiments, the second primer site is about 5 to about 25 nucleotides long. In some embodiments, the second primer site is about 5, 10, 15, 20, 25, 30, 35, 40, 45, or 50 nucleotides long. In some embodiments, the second primer site is about 20 nucleotides long. In some embodiments, the second primer site comprises at least 20%, at least 30%, at least 40%, at least 50%, at least 60%, at least 70%, at least 80%, at least 90%, at least 95%, at least 99%, or 100% sequence identity to SEQ ID NO: 6.

In some embodiments, the third primer site is about 5 to about 40 nucleotides long. In some embodiments, the third primer site is about 15 to about 30 nucleotides long. In some embodiments, the third primer site is about 5 to about 25 nucleotides long. In some embodiments, the third primer site is about 5, 10, 15, 20, 25, 30, 35, 40, 45, or 50 nucleotides long. In some embodiments, the third primer site is about 20 nucleotides long. In some embodiments, the third primer site is about 16 nucleotides long. In some embodiments, the third primer site comprises at least 20%, at least 30%, at least 40%, at least 50%, at least 60%, at least 70%, at least 80%, at least 90%, at least 95%, at least 99%, or 100% sequence identity to SEQ ID NO: 13.

In embodiments of the method, a single output oligonucleotide attached to the surface of the EV is formed comprising: (i) hybridizing the first hybridization sequence and the second hybridization sequence, hybridizing the third hybridization sequence and the oligonucleotide insert sequence, and hybridizing the fourth hybridization sequence and the fifth hybridization sequence so that the hybridized first detection sequence is in proximity to one end of the oligonucleotide insert sequence and the fifth hybridization sequence (of the third detection sequence) is in proximity to the other end of the oligonucleotide insert sequence; (ii) ligating the first hybridization sequence of the hybridized first detection sequence to the hybridized oligonucleotide insert; and (iii) ligating the hybridized oligonucleotide insert to the fifth hybridization sequence of the hybridized third detection sequence.

In embodiments, the method comprises amplifying the single output oligonucleotide using a strand-displacement primer that hybridizes to the third primer site and is used with a strand-displacement polymerase to amplify and displace the output oligonucleotide. In embodiments, the method further comprises amplifying the displaced output oligonucleotide using a first primer that hybridizes to the first primer site and a second primer that hybridizes to the second primer site. Amplification methods are described herein and are known in the art.

In embodiments of the methods herein, the methods further comprise detecting the amplified single output oligonucleotide. Detection methods are known in the art, and include quantitative PCR (qPCR), array binding and hybridization to labeled probes.

The steps in the above detection method may also be performed to determine the surface markers of an SMDA (such as, for example, and EV), to identify SMDAs that harbor combinations of surface markers, to detect populations of SMDAs having certain surface markers, and/or to detect or quantify multiple populations of SMDAs where each population has a specific set of surface markers. In embodiments of the method, the SMDA is an EV. EVs are described herein.

In embodiments, provided herein is a method of determining surface markers of a SMDA comprising contacting the SMDA with a plurality of unique binding reagents and an oligonucleotide insert. In embodiments, any one of the detection sequences comprises a third primer site. In embodiments, the third detection sequence comprises a third primer site.

In embodiments of the methods herein, the methods further comprise amplifying the single output oligonucleotide using a first primer that hybridizes to the first primer site and a second primer that hybridizes to the second primer site. Primers may be synthesized using known methods or purchased from a commercial supplier. Any suitable amplification technique can be used to amplify the output oligonucleotide (or amplicon), including but not limited to, PCR (Polymerase Chain Reaction), LCR (Ligase Chain Reaction), and isothermal amplification methods, e.g., helicase-dependent amplification, rolling circle amplification (RCA), 3SR (Self-Sustained Synthetic Reaction), transcription mediated amplification (TMA), nucleic acid sequence-based amplification (NASBA), signal mediated amplification of RNA technology, strand displacement amplification (SDA), loop-mediated isothermal amplification of DNA (LAMP), isothermal multiple displacement amplification, single primer isothermal amplification, and circular helicase-dependent amplification. In embodiments, the amplification technique is proximity ligation amplification (PLA) using RCA, which is known in the art, and disclosed in International Appl. No. PCT/US2015/030925, published as WO 2015/175856, which is incorporated by reference in its entirety.

In embodiments, the method comprises a first amplification step of the output oligonucleotide using strand-displacement amplification to amplify and displace the output oligonucleotide. In embodiments, the method comprises a second amplification step of the displaced output oligonucleotide using a first or forward primer that hybridizes to the first primer site and a second or reverse primer that hybridizes to the second primer site.

In embodiments of the methods herein, the methods further comprise detecting the amplified single output oligonucleotide. Detection methods are known in the art, and include quantitative PCR (qPCR), array binding and hybridization to labeled probes.

In embodiments, each sequencing read contains at least three barcode oligonucleotide sequences, which will be mapped to the identity of the binding reagent. In embodiments, the frequency of a specific combination of binding reagents or entities will be related to the abundance of the three markers (e.g., surface markers on an EV or epitopes on a protein). In embodiments, the abundance of a single marker (e.g., surface markers on an EV or epitopes on a protein) can be determined from the frequency with which the marker is identified from the barcode oligonucleotide sequencing results. In embodiments, multiple binding reagents or entities targeting the same marker can be compared using the barcode oligonucleotide sequencing results. For example, the highest affinity binding reagent or entity can be identified as the binding reagent most represented by its barcode in the sequencing data.

In embodiments, the sequencing is performed with high-throughput sequencing. In embodiments, the sequencing produces at least 10⁶ reads. In embodiments, the sequencing produces at least 10⁷ reads. In embodiments, the sequencing produces at least 10⁸ reads. In embodiments, the sequencing produces at least 10⁹ reads.

### Proximity Extension Strand Displacement

Provided herein are proximity-based extension assays (PEAs) combined with binding assay methodologies for measuring biomarkers, such as antibody-based immunoassays, and DNA-based methodologies (PCR and readout using either quantitative real-time PCR or next generation sequencing (NGS)), in, e.g., multiplexed formats, to simultaneously quantify the concentration of multiple protein biomarkers and/or SMDAs.

Provided herein are methods for analyzing a sample containing an analyte, e.g., an SMDA, multiplexed methods for analyzing a sample, methods for identifying a pairwise combination of binding moieties that can both be bound (or be simultaneously bound) to a binding target, for use in sandwich-type assays, and related compositions.

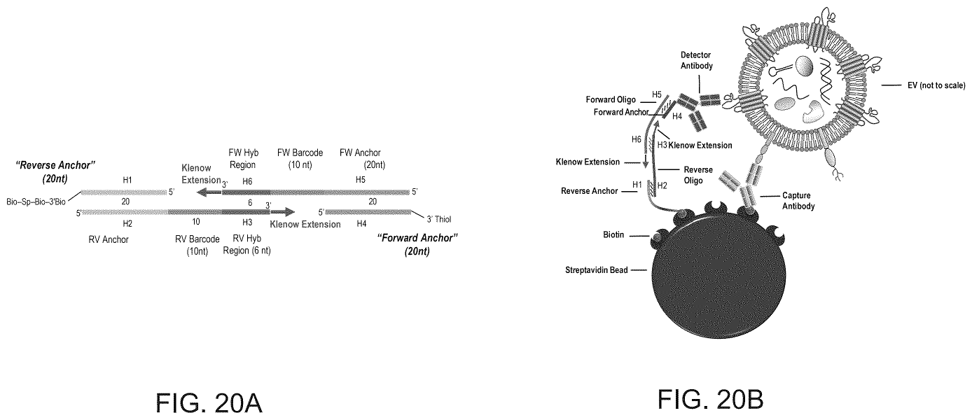

In embodiments, a proximity-based extension assay is any suitable assay that uses a binding moiety pair, wherein each member of the pair is bound (or simultaneously bound) to an analyte to bring two oligonucleotides in proximity such that hybridization between the oligonucleotides generates a template for an extension reaction. In some embodiments, the proximity-based extension assay is a proximity extension strand displacement (PESD) assay as disclosed in US20240409983A1 embodiments of which are illustrated in FIGS. 20A and 20B.

In embodiments, the present disclosure provides a method of determining surface markers of an SMDA comprising: contacting the SMDA with: (a) a capture reagent that binds to a first surface marker of the SMDA; (b) an anchoring reagent (“reverse anchor” in FIGS. 20A and 20B) comprising a first hybridization sequence (“H1” in FIGS. 20A and 20B); (c) a reverse oligonucleotide comprising: (i) a first barcode sequence; (ii) a second hybridization sequence (“H2” in FIGS. 20A and 20B); and (iii) a third hybridization sequence (“H3” in FIGS. 20A and 20B); (d) a binding reagent that binds to a second surface marker of the SMDA, wherein the binding reagent comprises a fourth hybridization sequence (“forward (FW) anchor or H4” in FIGS. 20A and 20B); and (e) a forward oligonucleotide comprising: (i) a fifth hybridization sequence (“H5” in FIGS. 20A and 20B); (ii) a second barcode sequence; and (iii) a sixth hybridization sequence (“H6” in FIGS. 20A and 20B). In embodiments, the first hybridization sequence and the second hybridization sequence are complementary. In embodiments, the third hybridization sequence and the sixth hybridization sequence are complementary. In embodiments, the fourth hybridization sequence and the fifth hybridization sequence are complementary

In embodiments, the method further comprises generating an output oligonucleotide by extending the hybridized reverse oligonucleotide and the hybridized forward oligonucleotide to form an output oligonucleotide. In embodiments, the output oligonucleotide generated comprises the first barcode sequence and the second barcode sequence.

In embodiments, the output oligonucleotide comprises a first primer site and a second primer site. In embodiments, the method further comprises releasing the output oligonucleotide by strand-displacement amplification followed by further amplification of the displaced output oligonucleotide. The method further comprises sequencing the output oligonucleotide to identify the first and second barcode sequences, thereby determining the surface markers of the SMDA.

In some embodiments, the reverse oligonucleotide comprises a first primer site. In embodiments, the reverse oligonucleotide comprises a 3′ to 5′ orientation or directionality. In embodiments, the reverse oligonucleotide comprises a 5′ to 3′ orientation or directionality. In some embodiments, the forward oligonucleotide comprises a second primer site. In embodiments, the forward oligonucleotide comprises a 5′ to 3′ orientation or directionality. In embodiments, the forward oligonucleotide comprises a 3′ to 5′ orientation or directionality. In some embodiments, the SMDA is an EV. In some embodiments, the SMDA is an exosome.

In embodiments, the hybridized reverse oligonucleotide and the hybridized forward oligonucleotide are extended by a strand-displacing DNA polymerase, and the extension product is released from the solid support by the strand-displacing DNA polymerase.

In embodiments, the reverse oligonucleotide comprises a first barcode sequence that identifies a first surface marker SMDA, and the forward oligonucleotide comprises a second barcode sequence that identifies a second surface marker of the SMDA. In embodiments, the first and second surface markers are the same. In embodiments, the first and second surface markers are different.

In embodiments, the forward oligonucleotide is about 20 to about 60 nucleotides long. In embodiments, the forward oligonucleotide is about 30 to about 50 nucleotides long. In embodiments, the forward oligonucleotide is about 15 to about 40 nucleotides long. In embodiments, the forward oligonucleotide is about 30, 32, 34, 36, 38 or 40 nucleotides long.

In embodiments, the reverse oligonucleotide is about 20 to about 60 nucleotides long. In embodiments, the reverse oligonucleotide is about 30 to about 50 nucleotides long. In embodiments, the reverse oligonucleotide is about 15 to about 40 nucleotides long. In embodiments, the reverse oligonucleotide is about 30, 32, 34, 36, 38 or 40 nucleotides long.

In some embodiments, the first primer site is about 5 to about 40 nucleotides long. In some embodiments, the first primer site is about 15 to about 30 nucleotides long. In some embodiments, the first primer site is about 5 to about 25 nucleotides long. In some embodiments, the first primer site is about 5, 10, 15, 20, 25, 30, 35, 40, 45, or 50 nucleotides long. In some embodiments, the first primer site is about 20 nucleotides long.

In some embodiments, the second primer site is about 5 to about 40 nucleotides long. In some embodiments, the second primer site is about 15 to about 30 nucleotides long. In some embodiments, the second primer site is about 5 to about 25 nucleotides long. In some embodiments, the second primer site is about 5, 10, 15, 20, 25, 30, 35, 40, 45, or 50 nucleotides long. In some embodiments, the second primer site is about 20 nucleotides long.

In some embodiments, the third primer site is about 5 to about 40 nucleotides long. In some embodiments, the third primer site is about 15 to about 30 nucleotides long. In some embodiments, the third primer site is about 5 to about 25 nucleotides long. In some embodiments, the third primer site is about 5, 10, 15, 20, 25, 30, 35, 40, 45, or 50 nucleotides long. In some embodiments, the third primer site is about 20 nucleotides long. In some embodiments, the third primer site is about 16 nucleotides long.

The hybridization sequences are complementary sequences that hybridize under stringent conditions. Hybridization under stringent conditions refers to conditions that permit hybridization of complementary nucleic acid sequences but preclude hybridization of sequences having significant mismatches. Stringent conditions are sequence-dependent and will vary with factors such as oligonucleotide length, base composition, and buffer composition. Generally, stringent conditions comprise hybridization in a buffer containing approximately 0.1-0.2×SSC (15-30 mM sodium chloride, 1.5-3 mM sodium citrate) at a temperature of about 50-68° C., followed by washes at the same or higher stringency (e.g., 0.1×SSC at 60-68° C.). In some embodiments, high stringency hybridization is carried out at about 65° C. in 0.1×SSC, 0.1% SDS, such that hybridization occurs only between perfectly complementary sequences, while lower stringency conditions (e.g., 2×SSC at 42° C.) may be used to detect sequences having up to a few mismatches.

In embodiments, hybridization sequences may also be referred to as “common overlap” or “overlap” sequences. In embodiments of the method, each hybridization sequence has a length of about 5-10 nucleotides. In embodiments, each hybridization sequence has a length of about 5-7 nucleotides. In embodiments, each hybridization sequence has a length of about 3-15 nucleotides. In embodiments, each hybridization sequence has a length of about 4-13 nucleotides. In embodiments, each hybridization sequence has a length of about 5-12 nucleotides. In embodiments, each hybridization sequence has a length of about 5-11 nucleotides. In embodiments, each hybridization sequence has a length of about 3, 4, 5, 6, 7, 8, 9, 10, 11, 12, 13, 14 or 15 nucleotides. In embodiments, each hybridization sequence is the same length. In embodiments, one or more of the hybridization sequences is a different length. In embodiments, the hybridization includes one or more non-canonical base-pairing.

In some embodiments, first hybridization sequence is about 10 to about 70 nucleotides long. In some embodiments, the anchor region is about 15 to about 30 nucleotides long. In some embodiments, the anchor region is about 5 to about 25 nucleotides long. In some embodiments, the anchor region is about 5, 10, 15, 20, 25, 30, 35, 40, 45, 50, 55, 60, 65, or 70 nucleotides long. In some embodiments, the anchor region is about 20 nucleotides long.

In some embodiments, the second hybridization sequence is about 10 to about 40 nucleotides long. In some embodiments, the second hybridization sequence is about 15 to about 30 nucleotides long. In some embodiments, the second hybridization sequence is about 5 to about 25 nucleotides long. In some embodiments, the second hybridization sequence is about 5, 10, 15, 20, 25, 30, 35, 40, 45, or 50 nucleotides long. In some embodiments, the second hybridization sequence is about 20 nucleotides long.

In some embodiments, the third hybridization sequence is 1 to about 40 nucleotides long. In some embodiments, the third hybridization sequence is about 15 to about 30 nucleotides long. In some embodiments, the third hybridization sequence is about 5 to about 25 nucleotides long. In some embodiments, the third hybridization sequence is about 3, 6, 9, 12 or 15 nucleotides long. In some embodiments, the second hybridization sequence is about 6 nucleotides long.

In some embodiments, the fourth hybridization sequence is about 10 to about 70 nucleotides long. In some embodiments, the fourth hybridization sequence is about 15 to about 30 nucleotides long. In some embodiments, the fourth hybridization sequence is about 5 to about 25 nucleotides long. In some embodiments, the fourth hybridization sequence is about 5, 10, 15, 20, 25, 30, 35, 40, 45, 50, 55, 60, 65, or 70 nucleotides long. In some embodiments, the fourth hybridization sequence is about 20 nucleotides long.

In embodiments, the fifth hybridization sequence is about 10 to about 40 nucleotides long. In some embodiments, the fifth hybridization sequence is about 15 to about 30 nucleotides long. In some embodiments, the fifth hybridization sequence is about 5 to about 25 nucleotides long. In some embodiments, the fifth hybridization sequence is about 5, 10, 15, 20, 25, 30, 35, 40, 45, or 50 nucleotides long. In some embodiments, the fifth hybridization sequence is about 20 nucleotides long.

In some embodiments, the sixth hybridization sequence is 1 to about 40 nucleotides long. In some embodiments, the sixth hybridization sequence is about 15 to about 30 nucleotides long. In some embodiments, the sixth hybridization sequence is about 5 to about 25 nucleotides long. In some embodiments, the sixth hybridization sequence is about 3, 6, 9, 12 or 15 nucleotides long. In some embodiments, the sixth hybridization sequence is about 6 nucleotides long.

In embodiments, the hybridization sequences are complexed to blocker oligonucleotides. In embodiments of the method, each blocker oligonucleotide has a length of about 5-20 nucleotides. In embodiments, each blocker oligonucleotide has a length of about 5-15 nucleotides. In embodiments, each blocker oligonucleotide has a length of about 5-10 nucleotides. In embodiments, each blocker oligonucleotide has a length of about 4-13 nucleotides. In embodiments, each blocker oligonucleotide has a length of about 5, 6, 7, 8, 9, 10, 11, 12, 13, 14 or 15 nucleotides. In embodiments, each blocker oligonucleotide is the same length. In embodiments, one or more of the blocker oligonucleotides is a different length.

In some embodiments, the reverse oligonucleotide is complexed with a first blocker oligonucleotide. In some embodiments, the first blocker oligonucleotide is hybridized to at least a portion of the third hybridization sequence. In some embodiments, the reverse oligonucleotide comprises a first blocker complement sequence. In some embodiments, the first blocker oligonucleotide is hybridized to the first blocker complement sequence. In some embodiments, the forward oligonucleotide is complexed with a second blocker oligonucleotide. In some embodiments, the second blocker oligonucleotide is hybridized to at least a portion of the sixth hybridization sequence. In some embodiments, the forward oligonucleotide comprises a second blocker complement sequence. In some embodiments, the second blocker oligonucleotide is hybridized to the second blocker complement sequence. In some embodiments, the reverse oligonucleotide comprises a third primer site. In some embodiments, the forward oligonucleotide comprises a third primer site.

In embodiments, the reverse or forward oligonucleotide can comprise one or more additional barcode sequences. In embodiments, the reverse or forward oligonucleotide can comprise one or more unique molecular identifiers.

A “barcode sequence” or “barcode oligonucleotide sequence,” as used herein, refers to a short nucleotide (typically from about 4 to about 40, 5 to about 30, about 6 to about 20, about 8 to about 18 or about 10 to about 16 nucleotides in length) that allows a corresponding nucleotide or molecule to be identified. In embodiments, the corresponding nucleotide or molecule is attached to the barcode sequence. In embodiments, the molecule is a peptide, a protein, a protein complex, an antibody, or a vesicle. In embodiments, the barcode sequence is a unique nucleotide identifiable by sequencing. In embodiments, the barcode sequence is hybridizable to a complementary detectable probe. In such embodiments, the complementary detectable probe hybridizes to the barcode sequence, allowing the corresponding nucleotide or molecule to be detected.

Provided herein are proximity-based assays that, in some non-limiting embodiments, employ capture surfaces and detection antibodies that engage in PESD to produce unique amplicons in the presence of a specific SMDA. Using these methods, the presence and/or identification of at least one surface marker in a sample can be detected and quantified by sequencing the resulting amplicons and/or performing PCR.

In some embodiments, a PESD assay of the present disclosure involves: hybridization of 2 overlapping oligonucleotides in close proximity to each other when a target analyte is present; one oligo is immobilized to a surface (e.g., a bead or culture plate well) and the other is attached to an antibody that specifically binds to the target analyte; extension of the overlapping, hybridized oligonucleotides to produce a double-stranded nucleic acid; release and amplification of the double-stranded nucleic acid; analysis of the resulting amplicons by PCR and/or NGS.

In some embodiments, a method of the present disclosure provides one or more of the following advantages: Extension and amplicon release can occur in the same step, which can reduce assay time as compared to methods that employ a separate step to release amplicons after their formation; antibody conjugates can share a common “tether,” which can reduce oligonucleotide cost and simplify conjugations. In some embodiments, unextended oligonucleotides (e.g., on bead or on detection conjugate) are not released into the sample/extension supernatant, which can reduce or effectively eliminate downstream PCR interference.

In some embodiments, during a PESD assay of the present disclosure, the extension and release steps are coupled because a single enzyme (e.g., a polymerase with strong strand displacement activity) performs both actions. This can improve the simplicity of the workflow and/or the efficiency of converting immunosandwiches (capture antibody-analyte-detection antibody) into amplifiable product. Using a polymerase with strong strand displacement activity (e.g., Klenow Fragment (3′→5′ exo-) to extend the oligos during primer extension (PE) can allow the strand displacement (SD) activity to selectively release the full-length amplicons of interest. Using the Klenow/SD PESD method can release more amplicon into the supernatant than other proximity-based extension assays (e.g., PEA) that release amplicon using enzymatic treatment (e.g., Proteinase K (ProK)).

In a specific embodiment, the methods of the invention can be used in a multiplexed format by binding a plurality of different analytes to a plurality of capture reagents for those analytes, the capture analytes being immobilized on coded bead, such that the coding identifies the capture reagent (and analyte target) for a specific bead. The method may further comprise counting the number of beads that have a bound analyte (using the assays described herein).

Alternatively or additionally, the capture reagents can be bound, directly or indirectly, to different discrete binding domains on one or more solid phases, e.g., as in a binding array wherein the binding domains are individual array elements, or in a set of beads wherein the binding domains are the individual beads, such that discrete assay signals are generated on and measured from each binding domain. If capture reagents for different analytes are immobilized in different binding domains, the different analytes bound to those domains can be measured independently. In one example of such an embodiment, the binding domains are prepared by immobilizing, on one or more surfaces, discrete domains of capture reagents that bind analytes of interest. Optionally, the surface(s) may define, in part, one or more boundaries of a container (e.g., a flow cell, well, cuvette, etc.) which holds the sample or through which the sample is passed. In a preferred embodiment, individual binding domains are formed on electrodes for use in electrochemical or electrochemiluminescence assays. Multiplexed measurement of analytes on a surface comprising a plurality of binding domains using electrochemiluminescence has been used in the Meso Scale Diagnostics, LLC, MULTI-ARRAY® and SECTOR® Imager line of products (see, e.g., U.S. Pat. Nos. 10,201,812, 7,842,246 and 6,977,722, the disclosures of which are incorporated herein by reference in their entireties).

### Blocker Oligonucleotides

The present disclosure provides methods for blocking undesirable, non-specific oligonucleotide-oligonucleotide hybridizations between the detection sequences (i.e., the 3′-, splint and 5′-conjugates) using blocker oligonucleotides. For example, the blocker oligonucleotides can hybridize to the first, second and/or third detection sequences such that non-specific, or non-complementary, detection sequences do not hybridize to each other. In embodiments, the blocker oligonucleotide-bound duplex region prevents the first detection (i.e., the 5′-conjugate) hybridization sequence from being hybridized to a non-complementary second detection sequence (i.e. splint conjugate), and/or from being extended and/or amplified. In embodiments, the blocker oligonucleotide-bound duplex region prevents the third detection (i.e., 3′-conjugate) hybridization sequence from being hybridized to a non-complementary second detection sequence (i.e. splint conjugate), and/or from being extended and/or amplified. In embodiments, hybridization of the blocker oligonucleotides reduces sequencing reads of the non-specific, non-complementary oligonucleotide hybridization interactions, which in turn increases sequencing reads of one or more output oligonucleotides. In embodiments, the first blocker oligonucleotide hybridizes to the 3′ end of the first detection sequence (i.e., the 5′ conjugate). In embodiments, the first blocker oligonucleotide hybridizes to at least a portion of the first hybridization sequence in the first detection sequence. In embodiments, the second blocker oligonucleotide hybridizes to at least a portion of the 5′ end of the third detection sequence (i.e., the 3′ conjugate). In embodiments, the second blocker oligonucleotide hybridizes to at least a portion of the fifth hybridization sequence in the third detection sequence. In embodiments, the second blocker oligonucleotide hybridizes to a second blocker-stabilizing sequence. In embodiments, the first blocker oligonucleotide comprises at least 20%, at least 30%, at least 40%, at least 50%, at least 60%, at least 70%, at least 80%, at least 90%, at least 95%, at least 99%, or 100% sequence identity to SEQ ID NO: 15. In some embodiments, the second blocker oligonucleotide comprises at least 20%, at least 30%, at least 40%, at least 50%, at least 60%, at least 70%, at least 80%, at least 90%, at least 95%, at least 99%, or 100% sequence identity to SEQ ID NO: 16.

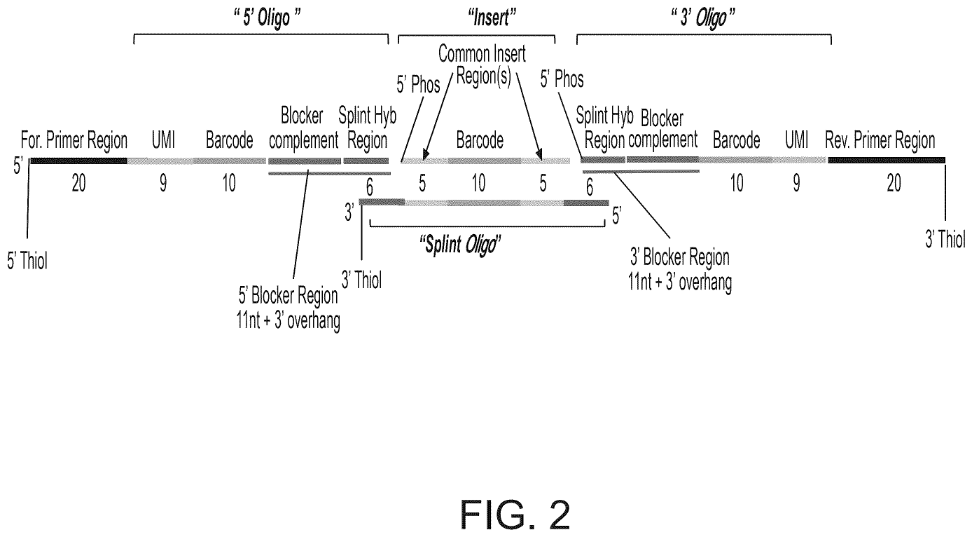

In embodiments, the first detection sequence comprises a first blocker complement sequence. In embodiments, the first blocker complement sequence is located between the first barcode sequence and the first hybridization sequence (5′ “Splint Hyb Region” in FIG. 2), as illustrated in FIG. 2. In embodiments, the first blocker complement sequence is about 5 to 50 nucleotides in length. In embodiments, the first blocker complement sequence is about 3 to 10 nucleotides in length. In embodiments, the first blocker complement sequence is about 5 to 15 nucleotides in length. In embodiments, the first blocker complement sequence is about 14 nucleotides in length. In embodiments, the first blocker complement sequence is about 11 nucleotides in length. In some embodiments, the first blocker complement sequence is about 15 to 35 nucleotides in length. In embodiments, the first blocker complement sequence is about 50 to 100 nucleotides in length. In some embodiments, the first blocker complement sequence is less than 10 nucleotides. In some embodiments the first blocker oligonucleotide is complementary to at least about 50%, about 60%, about 70%, about 80%, about 90% to the first blocker complement sequence. In embodiments the first blocker oligonucleotide hybridizes to at least a portion of the first blocker complement sequence and the first hybridization sequence.

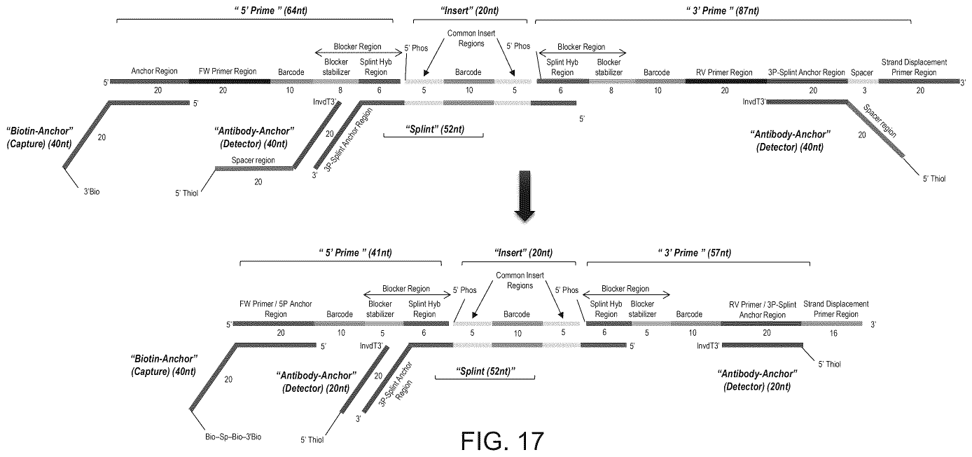

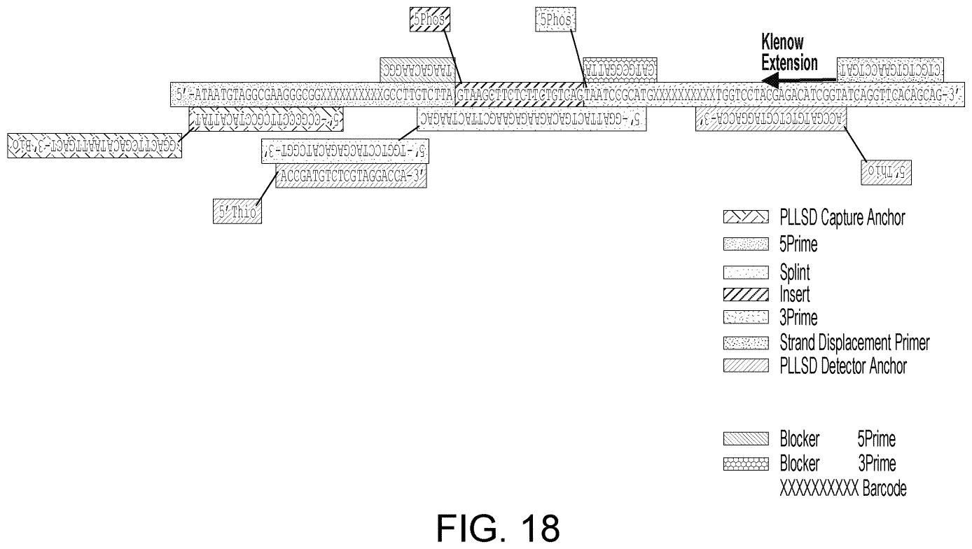

In some embodiments, the first blocker complement sequence comprises a first blocker-stabilizing sequence as exemplified in FIG. 17. In some embodiments, the first blocker complement sequence comprises at least a portion of the first hybridization sequence (5′ “Splint Hyb Region”) and a first blocker-stabilizing sequence. In some embodiments, the first blocker complement sequence comprises the full portion of the first hybridization sequence and a first blocker-stabilizing sequence. In embodiments, the first hybridization sequence comprises from about 3 to about 15 nucleotides. In embodiments, the first hybridization sequence comprises about 6 nucleotides. In embodiments, the first blocker-stabilizing sequence comprises from about 3 to about 15 nucleotides. In embodiments, the first blocker-stabilizing sequence comprises about 8 nucleotides. In embodiments, the first blocker-stabilizing sequence comprises about 5 nucleotides.

In embodiments, the third detection sequence comprises a second blocker complement sequence. In embodiments, the second blocker complement sequence is located between the third barcode sequence and the fifth hybridization sequence (3′ “Splint Hyb Region”), as illustrated in FIG. 2. In embodiments, the second blocker complement sequence is about 5 to 50 nucleotides in length. In embodiments, the second blocker complement sequence is about 3 to 10 nucleotides in length. In embodiments, the second blocker complement sequence is about 5 to 15 nucleotides in length. In embodiments, the first blocker complement sequence is about 14 nucleotides in length. In embodiments, the first blocker complement sequence is about 11 nucleotides in length. In some embodiments, the second blocker complement sequence is about 15 to 35 nucleotides in length. In embodiments, the second blocker complement sequence is about 50 to 100 nucleotides in length. In some embodiments, the second blocker complement sequence is less than 10 nucleotides. In some embodiments the second blocker oligonucleotide is complementary to at least about 50%, about 60%, about 70%, about 80%, about 90% to the second blocker complement sequence. In embodiments the second blocker oligonucleotide hybridizes to at least a portion of the second blocker complement sequence and the fifth hybridization sequence.

In some embodiments, the second blocker complement sequence comprises a second blocker-stabilizing sequence. In some embodiments, the second blocker complement sequence comprises at least a portion of the fifth hybridization sequence and a second blocker-stabilizing sequence as exemplified in FIG. 17. In some embodiments, the second blocker complement sequence comprises the full portion of the fifth hybridization sequence (3′ “Splint Hyb Region”) and a second blocker-stabilizing sequence. In embodiments, the fifth hybridization sequence comprises from about 3 to about 15 nucleotides. In embodiments, the fifth hybridization sequence comprises about 6 nucleotides. In embodiments, the second blocker-stabilizing sequence comprises from about 3 to about 15 nucleotides. In embodiments, the second blocker-stabilizing sequence comprises about 8 nucleotides. In embodiments, the second blocker-stabilizing sequence comprises about 5 nucleotides.

In some embodiments, “blocker-stabilizing sequences” are added to the first (5′ Prime) and third (3′ Prime) detection oligonucleotides, that allow for additional base pairing between the blocker complement sequences and barcode sequences, or the blocker complement sequences and hybridization sequences. In some embodiments, the hybridization region is 5-7 nt long, which would not create enough hybridization energy to stably duplex with a blocker under most conditions. Thus, additional neighboring bases are added to the blocker complement sequences that “stabilize” the blocker: detection duplex.

In embodiments, the nucleotide sequence of the first blocker complement sequence and the second blocker complement sequence are the same. In embodiments, the nucleotide sequence of the first blocker complement sequence and the second blocker complement sequence are substantially the same. In embodiments, the nucleotide sequence of the first blocker complement sequence and the second blocker complement sequence are 100% identical. In embodiments, the nucleotide sequence of the first blocker complement sequence and the second blocker complement sequence are at least about 80% identical, at least about 85% identical, at least about 90% identical, at least about 95% identical, or at least about 99% identical.

In embodiments, the present disclosure further describes a method for displacing the blocker oligonucleotides prior to forming the output oligonucleotide. In embodiments, the present disclosure describes a method for displacing the blocker oligonucleotides prior to hybridization of the first detection sequence (5′ conjugate) and the third detection sequence (3′ conjugate) to the second detection sequence (splint conjugate). In embodiments, the first and second blocker oligonucleotides are displaced from the first hybridization sequence and the fifth hybridization sequence prior to hybridization to the second detection sequence to form the output oligonucleotide. In embodiments, the first blocker oligonucleotide is displaced from the first hybridization sequence prior to hybridization of the first hybridization sequence to the second hybridization sequence. In embodiments, the first blocker oligonucleotide is displaced from the first blocker complement sequence and the first hybridization sequence prior to hybridization of the first hybridization sequence to the second hybridization sequence. In embodiments, the second blocker oligonucleotide is displaced from the fifth hybridization sequence prior to hybridization of the fifth hybridization sequence to the fourth hybridization sequence. In embodiments, the second blocker oligonucleotide is displaced from the second blocker complement sequence and the fifth hybridization sequence prior to hybridization of the fifth hybridization sequence to the fourth hybridization sequence.

In embodiments, hybridization of the blocker oligonucleotides can decrease the population (e.g., in terms of concentration) of non-specific, non-complementary oligonucleotide hybridization interactions by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to a reference biological sample to which the blocker oligonucleotides are not added. In embodiments, hybridization of the blocker oligonucleotides can increase the population of specific and complementary first, second, and third detection sequences that hybridize to each other by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to a reference biological sample to which the blocker oligonucleotides are not added.

In embodiments, hybridization of the blocker oligonucleotides can increase efficiency of identifying a surface marker on a SMDA by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more as compared to the efficiency of identifying the surface marker on the same SMDA in a reference biological sample to which the blocker oligonucleotides are not added. For example, in embodiments, hybridization of the blocker oligonucleotides can decrease sequencing reads of the non-specific, non-complementary oligo-oligo hybridizations by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to sequencing reads of the non-specific, non-complementary hybridizations from a reference biological sample to which the blocker oligonucleotides are not added. Consequently, hybridization of the blocker oligonucleotides can increase sequencing reads of one or more of a surface marker on the SMDA by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% about as compared to sequencing reads of the surface marker on the same SMDA from the reference biological sample to which the blocker oligonucleotides are not added.

In embodiments, the blocker oligonucleotides can be added to any solution or buffer described herein. In embodiments, the blocker oligonucleotides are added to the first, second, and third binding agent solutions prior to mixing. In embodiments, the blocker oligonucleotides can be added more than once (e.g., once, twice, three times or more) to the solutions or buffers described herein. In embodiments, the blocker oligonucleotide is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides in length. In embodiments, the blocker oligonucleotide is about 5 to about 20 nucleotides in length. In embodiments, the blocker oligonucleotide is about 10 to about 15 nucleotides in length. In embodiments, the blocker oligonucleotide is about 10 to about 20 nucleotides in length. In embodiments, the blocker oligonucleotide is about 5 to about 15 nucleotides in length. In embodiments, the blocker oligonucleotide is about 11 nucleotides in length.

In embodiments, the first blocker oligonucleotide comprises a 3′ overhang. In embodiments, the first blocker oligonucleotide comprises a 5′ overhang. In embodiments, the second blocker oligonucleotide comprises a 3′ overhang. In embodiments, the second blocker oligonucleotide comprises a 5′ overhang. The simplest DNA end of a double stranded molecule is called a blunt end. Blunt ends are also known as non-cohesive ends. In a blunt-ended molecule, both strands terminate in a base pair. Blunt ends are not always desired in biotechnology since when using a ligase to join two molecules into one, the yield is significantly lower with blunt ends. Non-blunt ends are created by various overhangs. An overhang is a stretch of unpaired nucleotides in the end of a DNA molecule. In embodiments, the overhangs are palindromic. In embodiments, the overhang comprises at least one nucleotide. In embodiments, the overhang comprises a single nucleotide. In embodiments, the single nucleotide is adenine and is created as a 3′ overhang by DNA polymerases known in the art. In embodiments, the product is joined with a linear DNA molecule with a 3′ thymine overhang. In embodiments, the overhang comprises two nucleotides. In embodiments, the overhang comprises three nucleotides. In embodiments, the overhang comprises one to five nucleotides.

In embodiments, any one of the blocker oligonucleotides of the present disclosure is in the form of a linear structure. In embodiments, any one of the blocker oligonucleotides of the present disclosure is in the form of a circular structure. In embodiments, any one of the blocker oligonucleotides of the present disclosure is in the form of a stem-loop structure.

In embodiments, the present disclosure provides a method, kit, or construct comprising one, two, three, four five, six, seven, eight, nine, or ten blocker oligonucleotides. In embodiments, the method, kit, or construct comprises two blocker oligonucleotides. In embodiments, the first blocker oligonucleotide comprises a single-stranded oligonucleotide sequence partially or completely complementary to the first detection sequence of the 5′-conjugate oligonucleotide. In embodiments, the first blocker oligonucleotide is at least 10%, at least 20%, at least 30%, at least 40%, at least 50%, at least 75%, at least 80%, at least 85%, at least 90%, at least 91%, at least 92%, at least 93%, at least 94%, at least 95%, at least 96%, at least 97%, at least 98%, or at least 99% complementary to the first detection sequence. In embodiments, the first blocker oligonucleotide is partially or completely complementary to the 3′ end of the first detection sequence. In embodiments, the first blocker oligonucleotide is at least 50%, at least 75%, at least 80%, at least 85%, at least 90%, at least 91%, at least 92%, at least 93%, at least 94%, at least 95%, at least 96%, at least 97%, at least 98%, or at least 99% complementary to the 3′ end of the first detection sequence. In embodiments, the blocker oligonucleotide is 100% (i.e., completely) complementary to the 3′ end of the first detection sequence. In embodiments, the second blocker oligonucleotide comprises a single-stranded oligonucleotide sequence partially or completely complementary to the third detection sequence of the 3′ conjugate oligonucleotide. In embodiments, the second blocker oligonucleotide is at least 10%, at least 20%, at least 30%, at least 40%, at least 50%, at least 75%, at least 80%, at least 85%, at least 90%, at least 91%, at least 92%, at least 93%, at least 94%, at least 95%, at least 96%, at least 97%, at least 98%, or at least 99% complementary to the third detection sequence. In embodiments, the second blocker oligonucleotide is partially or completely complementary to the 5′ end of the third detection sequence. In embodiments, the second blocker oligonucleotide is at least 50%, at least 75%, at least 80%, at least 85%, at least 90%, at least 91%, at least 92%, at least 93%, at least 94%, at least 95%, at least 96%, at least 97%, at least 98%, or at least 99% complementary to the 5′ end of the third detection sequence. In embodiments, the blocker oligonucleotide is 100% (i.e., completely) complementary to the 5′ end of the third detection sequence.

The blocker oligonucleotide can be produced by techniques known in the art. For example, in embodiments, the blocker oligonucleotide can be produced using chemical synthesis, in vitro expression from recombinant nucleic acid molecules, in vivo expression from recombinant nucleic acid molecules, or combinations thereof. The blocker oligonucleotide may also be produced by amplification of the undesirable nucleic acid, e.g., RT-PCR, asymmetric PCR, or rolling circle amplification, followed by incorporation of one or more non-natural nucleic acids. In embodiments, the blocker oligonucleotide comprises one or more (e.g., 1, 2, 3, 4, 5, 6, 7, 8, 9, 10, or more) modifications to its structure. For example, the modifications include one or more carbon moieties. In embodiments, the modifications include modified bases, e.g., 2′-O-methoxy-ethyl Bases (2′-MOE) (e.g., 2-MethoxyEthoxy A, 2-MethoxyEthoxy MeC, 2-MethoxyEthoxy G, and/or 2-MethoxyEthoxy T); 2′-O-Methyl RNA Bases (e.g., 2′-O-Methyl RNA Bases); Fluoro Bases (e.g., Fluoro C, Fluoro U, Fluoro A, and/or Fluoro G); 2-Aminopurine, 5-Bromo dU, deoxyUridine, 2,6-Diaminopurine (2-Amino-dA), Dideoxy-C, deoxyInosine, Hydroxymethyl dC, inverted dT, Iso-dG, Iso-dC, inverted Dideoxy-T, 3′-3′-inverted thymine, 5-Methyl dC, 5-Nitroindole, Super T (5-hydroxybutynl-2′-deoxyuridine), Super G (8-aza-7-deazaguanosine) and/or combinations thereof. In embodiments, the blocker oligonucleotide comprises DNA, RNA or LNA bases. In embodiments, the blocker oligonucleotide comprises a mixture of DNA, RNA and/or LNA bases.

In embodiments of the method, the plurality of binding reagents employed comprises: a. a first binding reagent comprising a first detection sequence that comprises a first hybridization sequence, and a first primer site, wherein the first hybridization sequence is hybridized, or complexed, to a first blocker oligonucleotide; b. a second binding reagent comprising a second detection sequence that comprises a second hybridization sequence, a third hybridization sequence, and a fourth hybridization sequence; and c. a third binding reagent comprising a third detection sequence that comprises a fifth hybridization sequence, and a second primer site, wherein the fifth hybridization sequence is hybridized, or complexed, to a second blocker oligonucleotide.

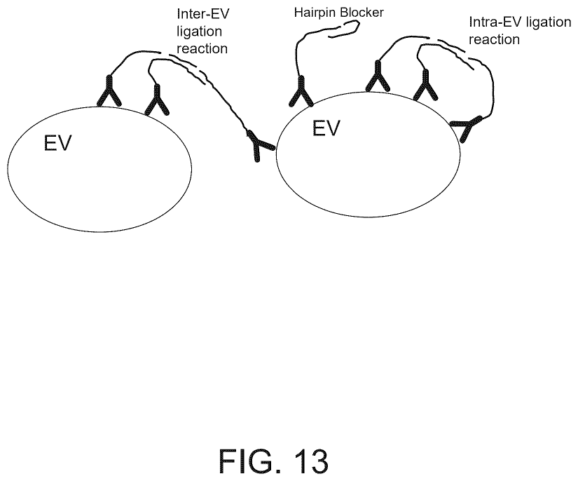

In embodiments, the method in the present disclosure employs use of hairpin blockers that hybridize and ligate to the free ends of oligonucleotides and eliminate the further possibility of inter-EV reactions in a sample (FIG. 13). In embodiments, the hairpin blockers are oligonucleotides with a circular structure. In embodiments, the hairpin blockers are oligonucleotides with a stem-loop structure. In embodiments, the sequence of the loop is perfectly complementary to the free oligonucleotide sequences. In embodiments, the 3′ end of the blocker is capped to prevent extension and several nucleotides at the 5′ end of the blocker compete for target binding with the 3′end of the free oligonucleotide sequence. In embodiments, the hairpin blockers are oligonucleotides with a linear structure. In embodiments, the sequence of the hairpin blocker is perfectly complementary to the free oligonucleotide sequences. In embodiments, the sequence of the hairpin blocker is partially complementary to the free oligonucleotide sequences. In embodiments, the sequence of the hairpin blocker is about 20%, 30%, 40%, 50%, 60%, 70%, 80%, 90%, or 100% complementary to the free oligonucleotide sequences. In embodiments, the hairpin blocker is about 5 to about 50 nucleotides in length. In embodiments, the hairpin blocker is about 10 to about 40 nucleotides in length. In embodiments, the hairpin blocker is about 20 to about 30 nucleotides in length. In embodiments, the hairpin blocker is about 5 to about 20 nucleotides in length. In embodiments, the hairpin blocker is about 5 to about 15 nucleotides in length.

In embodiments where EVs are released from the capture surface before the proximity reaction (PLL and/or PLLSD) the reaction conditions are tuned to favor the intra-EV ligation, rather than the inter-EV ligation to ensure that each sequencing amplicons comprises three barcodes from the same EV and combinations of barcodes from more than one EV (FIG. 13). In embodiments, the intra-EV ligation reaction can be favored by diluting the EVs so that the average spacing between EVs is high. In embodiments, the concentration of ligase is adjusted so that the intra-EV ligation reaction occurs quickly. In embodiments, hairpin blockers can be added to a reaction mixture at the same time as the ligase at a low concentration such that the ligation of these hairpin blockers to the unreacted oligos on EV-bound antibodies is slower than the formation of intra-EV ligation products. Over time most of the oligos on EV-bound antibodies will participate in intra-EV ligation reactions. In embodiments, those that are unavailable for intra-EV reaction, due to lack of suitable reaction partners, will be ligated by a hairpin blocker, thus terminated and unable to participate in inter-EV ligation reactions. In embodiments, the hairpin blockers could be added into the solution at high concentration in a second step after suitable time for most intra-EV reactions to take place. These blockers would then terminate all further proximity ligation reactions.

### Strand Displacement Amplification

In embodiments, the present disclosure provides a method of determining surface markers of a surface marker displaying agent (SMDA), comprising contacting a sample comprising a SMDA with: (a) an anchoring reagent hybridized to a first detection sequence, wherein the first detection sequence comprises a first unique barcode sequence and a first primer site; (b) a first binding reagent hybridized to a second detection sequence, wherein the second detection sequence comprises a second unique barcode sequence, wherein the first detection sequence and the second detection sequence comprise complementary nucleotide sequences; (c) a second binding reagent hybridized to a third detection sequence, wherein the third detection sequence comprises a third unique barcode sequence, a second primer site and a third primer site, wherein the second detection sequence and the third detection oligonucleotide comprise complementary nucleotide sequences; (d) a capture reagent; and (e) an oligonucleotide insert complementary to the second detection sequence, wherein if (i) the SMDA binds to the capture reagent, the first and second binding reagents, and (ii) the first detection sequence hybridizes to the second detection sequence, the second detection sequence hybridizes to the third detection sequence, and the oligonucleotide insert hybridizes to the second detection sequence, then a single-strand output oligonucleotide is generated that comprises the first, second, and third unique barcode sequences.

In embodiments, the first detection sequence comprises a first primer site and a first hybridization sequence. In embodiments, the second detection sequence comprises a second hybridization sequence, a third hybridization sequence, and a fourth hybridization sequence. In embodiments, the third detection sequence comprises a fifth hybridization sequence, a second primer site, and a third primer site. In embodiments, the first hybridization sequence and the second hybridization sequences are complementary. In embodiments, the third hybridization sequence and the oligonucleotide insert are complementary. In embodiments, the fourth hybridization sequence and the fifth hybridization sequences are complementary. Thus, in embodiments, the first and third detection sequences can each hybridize to the second detection sequence. In embodiments, after the first and third detection sequences hybridize to the second detection sequence, ligase is added to ligate the first detection sequence, the oligonucleotide insert, and the third detection sequence into a single output oligonucleotide. In embodiments, after ligation, strand-displacement polymerase is added to remove any gaps in the ligated sequence, and to amplify and release the output oligonucleotide using the third primer site. In embodiments, the ligase and strand displacement polymerase are added at the same time. In embodiments, after ligation, the strand-displacement polymerase is added to remove any gaps in the ligated sequence, and to amplify and release the output oligonucleotide using the third primer site. In embodiments, after displacement, a second polymerase is added to further amplify the output oligonucleotide using the first primer site and the second primer site. In embodiments, the amplification further comprises adding one or more sequencing primer sites to the ends of the output oligonucleotide. In embodiments, sequencing is performed using the sequencing primer sites. In embodiments, sequencing is performed using the first primer site and the second primer site.

In embodiments, the method comprises generating a double-strand output oligonucleotide from the single-strand output oligonucleotide by binding a primer complementary to the third primer site and extending the primer to form a second strand complementary to the single-strand output oligonucleotide. In embodiments, the third detection sequence in the present disclosure comprises a third primer site. In embodiments, the method employed in the present disclosure comprises (i) binding a strand displacement primer to the third primer site and extending the single-strand output oligonucleotide to form a double-strand output oligonucleotide, and (ii) releasing a single-strand output oligonucleotide. In the context of strand-displacement, the terms “released” and “displaced” may be used interchangeably throughout the application and have the same meaning.

Strand displacement amplification (SDA) is an isothermal method of nucleic acid amplification in which extension of primers, displacement of single stranded extension products, annealing of primers to the extension products (or the original target sequence) and subsequent extension of the primers occurs concurrently in the reaction mix. This is in contrast to the PCR, in which the steps of the reaction occur in discrete phases or cycles as a result of the temperature constraints of the reaction. In some embodiments, during the annealing and elongation of primers on the template output oligonucleotide, a single-strand break (nick) is introduced in the nucleotide sequence of the annealed primer to the third primer site. The resulting nicks serve as the initiation sites for the synthesis of a new output oligonucleotide, and the output oligonucleotide initially synthesized as a result of primer elongation is displaced by the output oligonucleotide-displacing activity of a polymerase. The result is a double-stranded output oligonucleotide and displaced single-stranded output oligonucleotide. The displaced output oligonucleotide strand is primed by a complementary oligonucleotide primer, which again leads to primer elongation, the introduction of a break in the nucleotide sequence of the primer, and the displacement of the new output oligonucleotide strand with the simultaneous formation of a free double-stranded output oligonucleotide.

SDA is based upon 1) the ability of a restriction endonuclease to nick the unmodified strand of a hemiphosphorothioate form of its double stranded recognition site and 2) the ability of certain polymerases, such as the exo-Klenow DNA polymerase, to initiate replication at the nick and displace the downstream non-template strand. Production of each new copy of the target sequence consists of five steps: 1) binding of primers to an original target sequence or a displaced single-stranded extension product previously polymerized, 2) extension of the primers by exonuclease deficient (exo-) Klenow polymerase incorporating an α-thio deoxynucleoside triphosphate, 3) nicking of a hemiphosphorothioate double stranded restriction site, 4) dissociation of the restriction enzyme from the nick site, and 5) extension from the 3′ end of the nick by exo-Klenow with displacement of the downstream non-template strand. Nicking, polymerization and displacement occur concurrently and continuously at a constant temperature because extension from the nick regenerates another nickable restriction site. When primers which hybridize to a strand of a double stranded target sequence are used, amplification is exponential, as the sense and antisense strands serve as templates for the opposite primer in subsequent rounds of amplification. SDA is described by G. T. Walker, et al. (1992a. Proc. Natl. Acad. Sci. USA 89, 392-396 and 1992b. Nuc. Acids. Res. 20, 1691-1696). Examples of restriction enzymes which nick their double stranded recognition sites when an α-thio dNTP is incorporated are HincII, HindII, AvaI, NciI and Fnu4HI. All of these restriction enzymes and others which display the required nicking activity are suitable for use in SDA.

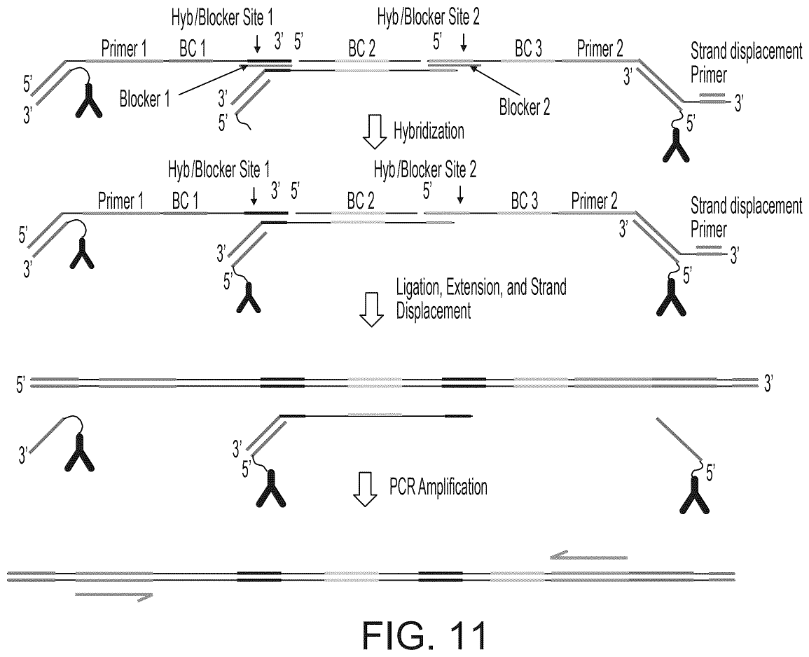

In embodiments, the 3′ and/or 5′ conjugate oligonucleotide comprises a third primer site. In embodiments, following ligation between the oligonucleotide insert, the first and the third detection sequence to form the output oligonucleotide, a third primer binds to the third primer site and extends the single-strand output oligonucleotide to form a double-strand output oligonucleotide, and simultaneously releases the double-strand output oligonucleotide from the splint conjugate, as shown in FIG. 11. In embodiments, the 3′ end of the third detection sequence of the third binding reagent comprises the third primer.

### CRC Markers

In embodiments of the methods disclosed herein, the methods comprise performing an assay to detect colorectal cancer (CRC)-derived EVs that comprise both CEA and CD73 (CEA+/CD73+ EVs). In embodiments, the method comprises a 3-marker proximity assay, such as a proximity ligation assay (PLA) or a proximity extension assay (PEA) that employs a first binding reagent that is a capture reagent, and two additional binding agents that are proximity probes, wherein each of the three binding reagents binds to one of CEA, CD73, and a tetraspanin.

In embodiments, a three-marker assay for intact EVs is disclosed herein is used, using a first binding reagent that binds CEA or CD73, and a second binding reagent that binds CEA or CD73, and a third binding reagent that binds a common EV marker (tetraspanin), such as CD63, CD81, or CD9.

Three-marker proximity assays and proximity probes are well known in the art. Proximity probes comprise an analyte binding domain (e.g., and antibody or antibody fragment) linked directly or indirectly to a functional domain (e.g., an oligonucleotide). Any of the 3-marker proximity assay formats disclosed in WO2019222708, WO2020086751, and WO2022051481 can be used to detect CEA+/CD73+ EVs if the format uses binding reagents that bind to CEA, CD73, and a tetraspanin. In embodiments, the binding reagents employed in an assay to detect CEA+/CD73+ EVs employ any of the binding reagent configurations listed in Table 1. In embodiments, the binding reagents employed in an assay to detect CRC-derived EVs employ first, second and third binding reagents that bind to any surface marker on the EV selected from: CD9, CD73, CD324, CD325, CD326, CD13, CD66a, CD66e, CD10, CD31, CD36, CD141, CD14, CD54, CD26, MHC Class II molecules, and FLT1, in any combination thereof. In embodiments, the first binding reagent binds to a surface marker on the EV selected from: CD9, CD73, CD324, CD325, CD326, CD13, CD66a, CD66e, CD10, CD31, CD36, CD141, CD14, CD54, CD26, MHC Class II molecules, and FLT1. In embodiments, the second binding reagent binds to a surface marker on the EV selected from: CD9, CD73, CD324, CD325, CD326, CD13, CD66a, CD66e, CD10, CD31, CD36, CD141, CD14, CD54, CD26, MHC Class II molecules, and FLT1. In embodiments, the third binding reagent binds to a surface marker on the EV selected from: CD9, CD73, CD324, CD325, CD326, CD13, CD66a, CD66e, CD10, CD31, CD36, CD141, CD14, CD54, CD26, MHC Class II molecules, and FLT1. In embodiments, the capture reagent binds to a surface marker on the EV selected from: CD9, CD73, CD324, CD325, CD326, CD13, CD66a, CD66e, CD10, CD31, CD36, CD141, CD14, CD54, CD26, MHC Class II molecules, and FLT1.

TABLE 1

| | first binding reagent | second binding reagent | third binding reagent |
|---|---|---|---|
| Configuration 1 | CEA | CD81 or other TTSP | CD73 |
| Configuration 2 | CEA | CD73 | CD81 or other TTSP |
| Configuration 3 | CD73 | CEA | CD73 |
| Configuration 4 | CD73 | CD81 or other TTSP | CEA |
| Configuration 5 | CD81 or other TTSP | CD73 | CEA |
| Configuration 6 | CD81 or other TTSP | CEA | CD73 |
| Configuration 7 | CD73 | CD81 or other TTSP | CD73 |

In embodiments, the present disclosure employs method for detecting colorectal tumor-derived SMDA in a sample from a subject suspected of having a colorectal tumor or suspected of having CRC, comprising detecting at least two CRC-related markers on the SMDA in the sample. In embodiments, the method comprises detecting three CRC-related markers on the SMDA in the sample. In embodiments, any combination of the surface markers selected from: CD9, CD73, CD324, CD325, CD326, CD13, CD66a, CD66e, CD10, CD31, CD36, CD141, CD14, CD54, CD26, MHC Class II molecules, and FLT1, is enriched in a CRC sample from a subject relative to a healthy subject.

In embodiments, the present disclosure employs a method for determining eligibility of a subject to participate in a clinical trial of a therapeutic drug for preventing or delaying CRC, conducting a clinical trial of CRC, distinguishing a subject afflicted with CRC, or treating CRC in a subject in need thereof. In embodiments, the methods comprise: (a) determining the eligibility of the subject for the clinical trial based on a measurement of at least two CRC-related markers on a colorectal tumor-derived SMDAs in a sample from the subject; (b) identifying, based on the measurement of the at least two CRC-related markers, the subject as (i) afflicted with CRC, or (ii) without CRC; and (c) administering the therapeutic drug to the subject.

In embodiments, the present disclosure employs a method of monitoring response to treatment for colorectal CRC in a subject, the method comprising: (a) determining, based on a first measurement of at least two CRC-related markers on colorectal tumor-derived SMDAs in a sample of the subject, wherein the first measurement is obtained prior to administration of a treatment regimen for CRC and a second measurement of the at least two CRC-related markers in the subject at one or more time points after administration of the treatment regimen for CRC has been initiated, that the subject is responding positively to the CRC treatment regimen, and (b) continuing to administer the treatment regimen for CRC to the subject.

In embodiments, the present disclosure employs a method of identifying colorectal CRC in a human, the method comprising: obtaining measured levels of three CRC-related markers on colorectal tumor-derived SMDAs in a sample suspected of containing SMDAs from the human, wherein the sample is selected from the group consisting of whole blood, serum, plasma, and combinations thereof, and wherein the levels of the three CRC-related markers are obtained by a multimarker immunoassay comprising contacting the sample with a first oligonucleotide-conjugated capture entity that binds a first CRC-related surface marker of the SMDA, a second oligonucleotide-conjugated splint entity that binds a second CRC-related surface marker of the SMDA, a third oligonucleotide-conjugated staple entity that binds a third CRC-related surface marker of the SMDA, and a surface.

### Samples

In embodiments of the methods of the invention, SMDAs of interest are isolated from samples using the methods of the invention. In embodiments of the invention, the sample comprises the SMDAs of interest and unwanted components. In embodiments of the methods of the invention, before contacting the sample with a surface and selectively binding the SMDAs of interest, the sample, e.g., mammalian fluid, secretion, or excretion, is purified by, for instance, differential centrifugation, ultrafiltration, size-exclusion chromatography, immuno-affinity, precipitation, or a combination thereof. In embodiments, the unwanted components are soluble in the sample and/or the washing fluid. Further unwanted components can include, but are not limited to, SMDAs that do not have the marker that the capture reagent binds to, the marker that the binding reagent binds to, or both. In embodiments, the unwanted components include SMDAs that bind to the capture reagent, but not the binding reagent. In the methods of the invention, SMDAs that bind to the capture reagent, but not the binding reagent, will be eluted following releasing the capture reagent from the surface. In embodiments, the SMDA is a cell. In embodiments, the SMDA is a virus or viral particle. In embodiments, the SMDA is an organelle. In embodiments, the SMDA is a vesicle. In embodiments, the SMDA is an EV or exosome.

In embodiments of the methods of the invention, EVs of interest are isolated from samples using the methods of the invention. In embodiments of the invention, the sample comprises the EVs of interest and unwanted components. In embodiments of the methods of the invention, before contacting the sample with a surface and selectively binding the EV of interest, the sample, e.g., mammalian fluid, secretion, or excretion, is purified by, for instance, differential centrifugation, ultrafiltration, size-exclusion chromatography, immuno-affinity, precipitation, or a combination thereof. In embodiments, the unwanted components are soluble in the sample and/or the washing fluid. Further unwanted components can include, but are not limited to, EVs that do not have the marker that the capture reagent binds to, the marker that the binding reagent binds to, or both. In embodiments, the unwanted components include EVs that bind to the capture reagent, but not the binding reagent. In the methods of the invention, EVs that bind to the capture reagent, but not the binding reagent, will be eluted following releasing the capture reagent from the surface.

In embodiments of the methods of the invention, the sample comprises EVs produced from a cell differentiated from a cell-line, differentiated from an induced pluripotent stem cell, a primary cell, or a combination thereof. Samples further include cell supernatants, such as those from neuronal and astrocyte cultures, which include at least the following: human cortical neurons differentiated from induced pluripotent stem cells (iPSC) and from the HCN-2 cell line, adult NPC derived neurons, and adult primary neurons, as well as mature astrocytes differentiated from iPSC and primary human astrocytes. In embodiments, samples include supernatants from oligodendrocytes derived from iPSC cells, which are commercially available, and from cell lines such as HOG or M03.13 which can be differentiated to mature oligodendrocytes using established protocols. Samples further include iPSC derived microglia, which are commercially available, as well as primary microglia which can be expanded in culture. Non-limiting examples of cell lines include MOLT-4 (differentiated or undifferentiated), Jurkat, HL60 (differentiated or undifferentiated), U-937 (differentiated or undifferentiated), HDLM-2, THP-1 (differentiated or undifferentiated), GA10, Ramos, HUVEC, PANC-1, Expi293, HaCat, HCT-15, H-2228, peripheral blood mononuclear cells (PBMCs), KU-812, MC-04, HT-1376, TT, HCT-1116, MCF-7, Calu-3, and the like. Exemplary monocytic cell lines include THP-1, differentiated THP-1, HL60, and differentiated HL60. An exemplary NK cell line is NK92. Exemplary T cell lines include Jurkat and Molt-4. An exemplary B cell line is GA-10. Exemplary endothelial cell lines include HUVEC and differentiated HUVEC. Exemplary hepatocytic cell lines include HepG2 and differentiated HepG2. Exemplary epithelial cell lines include A549, A431, Caco-2, HT29, LNCap, SKOV3, SW480, PC3, MDMB-468, MDMB-231, MCF7, HT-1376, PANC-1, HCT15, Calu-3, Skov3, Bewo, K562, and HeLa. Further additional cell lines include, e.g., HT-29 sARPE-19, SH-SY5Y, and U87-MG.

In embodiments, the samples comprise EVs produced from a T cell, a B cell, a dendritic cell, an NK cell, a monocyte, a macrophage, a granulocyte, a platelet, an erythrocyte, an endothelial cell (e.g., an aortic endothelial cell), an epithelial cell, a stem cell precursor cell, a mesenchymal stem cell, a hematopoietic stem cell, a leukocyte, a senescent cell, an adipose cell, a hepatocyte, a myocyte, or a skeletal muscle cell. T cells include, e.g., helper T cells, such as the subtypes Th1, Th2, Th9, Th17, Th22, and Tfh; regulatory T cells; killer T cells; γδ TCR+ T cells; and natural killer T cells. Adipose cells include, e.g., normal adipocytes, diabetic adipocytes, omental adipocytes, MSC-derived adipocytes, preadipocytes, and omental preadipocytes.

In embodiments, the sample comprises tissue explants in suspension culture. In embodiments, the tissue explant comprises adipocytes or monocytes.

In embodiments of the invention, the sample is a mammalian fluid, secretion, or excretion. In embodiments, the sample is a purified mammalian fluid, secretion, or excretion.

In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions and excretions. In embodiments, the sample is cerebrospinal fluid.

In embodiments, the sample is obtained from an individual, e.g., a human. In embodiments, the sample comprises a plasma sample from an individual. In embodiments, the sample is obtained from a healthy individual. In embodiments, the sample is obtained from an individual having or at risk of a disease. In embodiments, the disease is a cardiovascular disease, a viral infection, cancer, or combination thereof. For instance, the sample can include a plasma sample from a healthy individual, an individual having but not treated for CRC, and an individual with CRC and treated with a therapeutic drug. In embodiments, a library comprising multiple pools of binding reagents provided herein is used to detect surface markers described herein, the binding reagents in the pools configured such that any combination of markers in the sample can be detected.

In embodiments, the sample comprises purified EVs. Methods of purification include, but are not limited to, precipitation, ultracentrifugation, size exclusion chromatography, ultrafiltration, or affinity purification. In embodiments, the affinity purification may be performed with magnetic or non-magnetic beads. In embodiments, the sample provided herein is initially depleted of undesirable EVs. In embodiments, plasma samples are depleted from platelet-derived EVs. In embodiments, anti-CD41 and CD61 beads are employed to deplete platelet-derived EVs.

Biological samples that may be analyzed include, but are not limited to, physiological samples and/or samples containing suspensions of cells, such as mucosal swabs, tissue aspirates, tissue homogenates, cell cultures, and cell culture supernatant, including cultures of eukaryotic and prokaryotic cells. In embodiments, cells are removed, before contacting the surface with EVs, by, for instance centrifugation or filtration.

In embodiments, different cells are identified and distinguished from each other, based on the EVs detected using the present methods. For example, the present methods may be used to distinguish between differentiated and undifferentiated cells, or between diseased and normal (healthy) cells, based on the EVs secreted by each type of cell. The present methods may also be used to determine the growth stage or stimulated state of a cell or cell population. For example, the present methods may be used to compare multiple growth or stimulation conditions on a single cell line. Advantageously, the present methods facilitate comparison of EV secretion in multiple cell lines and reduce potentially tedious sample preparation. In embodiments, the detection of EVs is used to diagnose or assess risk of a disease in a subject.

In embodiments, EVs detected by the present methods are used to assess the presence of different cell types in a sample. For example, detection of a surface marker known to be on EVs from a particular cell type in a sample would indicate presence of that cell type in the sample. Thus, in embodiments, the present methods are used to detect the presence of contaminating cell types in a sample. For example, a sample derived from CNS cells would not be expected to have a surface marker for an endothelial cell; however, if an EV with a marker for a non-CNS cell, e.g., an endothelial cell-specific surface marker, is detected, the sample may be contaminated with non-CNS cells. Non-limiting examples of surface markers that can be used to assess for the presence of endothelial cells in a sample include endoglin, thrombomodulin, PECAM, ICAM-1, ICAM-3, and CD276. Non-limiting examples of surface markers that can be used to assess for the presence of platelet cells in a sample include P-selectin. Non-limiting examples of surface markers that can be used to assess for the presence of lymphoid cells in a sample include CD3, CD4, CD8, and CD19. Non-limiting examples of surface markers that can be used to assess for the presence of myeloid cells in a sample include CD15 and CD66b. Non-limiting examples of surface markers that can be used to assess for the presence of epithelial cells in a sample include EpCAM, EGFR, EphA2, and E-cadherin.

In embodiments, the relative abundance of surface markers on SMDAs is determined using a library comprising multiple pools of binding reagents as provided herein, wherein each pool contains a specified number of a same binding reagent. In embodiments, the relative abundance of EVs in a sample can be measured by using a plurality of different capture reagents to capture different EVs expressing different markers, then detecting each type of captured EVs with the same detection reagent to a common marker on the different EVs. In embodiments, the relative abundance of EVs in a sample can be measured by using the same capture reagent to capture different EVs expressing a common marker, then using a plurality of different detection reagents to determine the different markers expressed by the EVs.

Samples may be obtained from a single source described herein, or may contain a mixture from two or more sources, e.g., two or three or four cell lines or other sources described herein.

In embodiments, the biological sample is obtained from a subject, e.g., a human. In embodiments, the biological sample comprises a plasma sample from a subject. In embodiments, the biological sample is obtained from a healthy subject. In embodiments, the biological sample is obtained from a subject suspected of having a colorectal tumor or suspected of having CRC. In embodiments, the subject is suspected of having late-stage CRC or stage IV CRC. In embodiments, the subject is diagnosed with having a colorectal tumor, CRC, late-stage CRC, or stage IV CRC. In embodiments, the subject is receiving treatment for CRC. In embodiments, the subject previously had CRC and was previously classified as being in remission.

“Stage IV” colorectal cancer as used herein includes Stages IV A, IV B, and IV C of the CRC TNM staging system established by the American Joint Committee on Cancer (AJCC) and encompasses colorectal cancer that (i) might or might not have grown through the wall of the colon or rectum (Any T), (ii) might or might not have spread to nearby lymph nodes (Any N), and (iii) has spread to 1 distant organ (such as the liver or lung) or distant set of lymph nodes, but not to distant parts of the peritoneum (the lining of the abdominal cavity) (Mla), or has spread to more than 1 distant organ (such as the liver or lung) or distant set of lymph nodes, but not to distant parts of the peritoneum (the lining of the abdominal cavity) (M1b), or has spread to distant parts of the peritoneum (the lining of the abdominal cavity), and may or may not have spread to distant organs or lymph nodes (M1c).

“Late stage” colorectal cancer as used herein includes Stages III and IV of the CRC TNM staging system established by the American Joint Committee on Cancer (AJCC).

### Surface Comprising Capture Reagent and Anchoring Reagent/Adaptor Oligos

In embodiments of the disclosure, a sample comprising a SMDA of interest is contacted with a surface, wherein the surface comprises a capture reagent; an anchoring reagent; a first binding reagent comprising a first unique barcode sequence; and a second binding reagent comprising a second unique barcode sequence. In embodiments, the first and second binding reagent comprise complementary nucleotide sequences.

In embodiments, the anchoring reagent comprises a third unique barcode sequence, wherein the anchoring reagent comprises a nucleotide sequence complementary to the second binding reagent nucleotide sequence. In some embodiments, the anchoring reagent comprises an anchoring oligonucleotide. In embodiments the anchoring oligonucleotide is about 10 to about 70 nucleotides long. In embodiments the anchoring oligonucleotide is about 10 to about 30 nucleotides long. In embodiments, the anchoring oligonucleotide is about 20 nucleotides long. In embodiments, the “first” and “second” binding reagents may be used interchangeably.

In embodiments, if the SMDA binds to the capture reagent, the first and second binding reagents, and if the anchoring reagent nucleotide sequence hybridizes to the second binding reagent nucleotide sequence, then a single-strand output oligonucleotide is generated that comprises the barcode oligonucleotide sequences of each of the anchoring reagent, and the first and second binding reagents.

In embodiments, the single-strand output oligonucleotide is extended by strand-displacement amplification to form a double-strand output oligonucleotide. In embodiments, the double-strand output oligonucleotide is amplified, and the amplified output oligonucleotide is sequenced to identify the barcode oligonucleotide sequences, thereby determining at least three unique surface markers of the SMDA.

In embodiments, at least a portion of the nucleotide sequence of the first binding reagent is hybridized to a blocker sequence. In embodiments, at least a portion of the nucleotide sequence of the anchoring reagent is hybridized to a blocker sequence. In embodiments, the nucleotide sequence of the first binding reagent comprises a first connecting sequence hybridized to a complementary sequence on the first binding reagent. In embodiments, the nucleotide sequence of the second binding reagent comprises a second connecting sequence hybridized to a complementary sequence on the second binding reagent. In embodiments, the anchoring reagent comprises a third connecting sequence. In embodiments, the first connecting sequence, the second connecting sequence, and the third connecting sequence are unique. In embodiments, the first connecting sequence, the second connecting sequence, and the third connecting sequence are the same. In embodiments, the first connecting sequence, the second connecting sequence, and the third connecting sequence are substantially the same. In embodiments, the first connecting sequence, the second connecting sequence, and the third connecting sequence comprise about 80%, 85%, 90%, 95%, 96%, 97%, 98%, 99%, or 100% identical sequences.

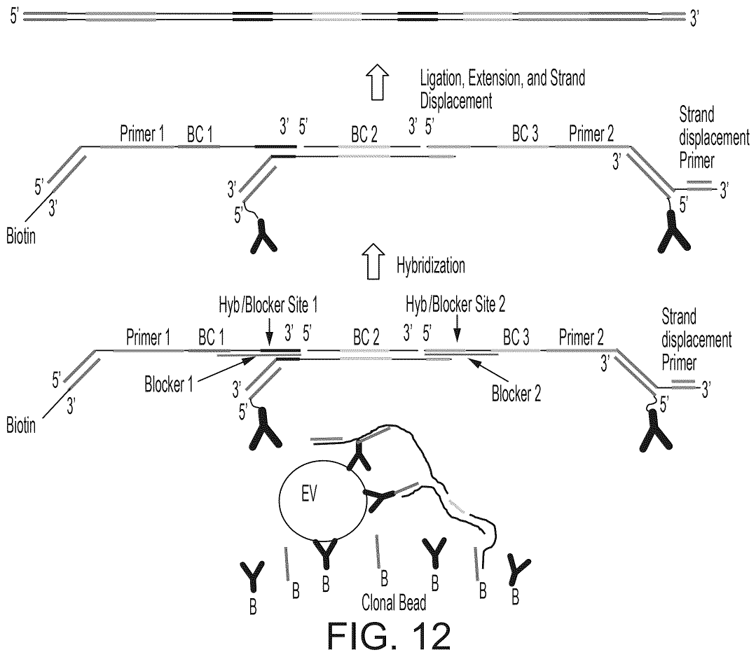

In embodiments, the present disclosure employs a method of determining surface markers of a SMDA, as illustrated in FIG. 12, comprising: a) contacting a sample comprising a SMDA with (i) a first binding reagent comprising a first unique barcode sequence and a second binding reagent comprising a second unique barcode sequence, wherein the first and second binding reagent comprise complementary nucleotide sequences; (ii) a capture reagent; and (iii) an anchoring reagent, wherein the anchoring reagent comprises a third unique barcode sequence, wherein the anchoring reagent comprises a nucleotide sequence complementary to the second binding reagent nucleotide sequence. In embodiments, the capture reagent is specific to a first common EV marker. In embodiments, the first binding reagent is specific to a second common EV marker. In embodiments, the second binding reagent is specific to a third common EV marker. In embodiments, the capture reagent is specific to a tumor-derived EV marker. In embodiments, the capture reagent is specific to a CRC-related EV marker. In embodiments, the first binding reagent is specific to a second tumor-derived EV marker. In embodiments, the first binding reagent is specific to a second CRC-related EV marker. In embodiments, the second binding reagent is specific to a third tumor-derived EV marker. In embodiments, the second binding reagent is specific to a third CRC-related EV marker.

In embodiments, a sample comprising an SMDA of interest is contacted with a surface, wherein the surface comprises a releasably bound capture reagent and an anchoring reagent. The term “contacting” has its ordinary meaning to one of skill in the art. Methods of contacting samples, e.g., liquids, solids, gels, etc., are known to those of ordinary skill in the art. In embodiments, the capture reagent and the anchoring reagent are bound to the same surface. In embodiments, the capture reagent and the anchoring reagent are releasably bound to the same surface. In embodiments, the capture reagent and the anchoring reagent are bound to separate surfaces. In embodiments, the capture reagent and the anchoring reagent are releasably bound to separate surfaces.

In embodiments of the methods of the invention, the capture reagent is releasably bound to the surface by a labile linker. In embodiments, the labile linker is a heat-labile, a photolabile, or a chemically labile linker. In additional embodiments, the labile linker is an oligonucleotide that is complementary to an oligonucleotide bound to the surface or is an oligonucleotide comprising a restriction site cleavable by a restriction endonuclease. In embodiments, the labile linker is a small molecule that binds to a protein on the surface. In embodiments, the capture reagent is biotinylated, and the surface is coated with streptavidin. The surface can be, for example, an MSD plate electrode or a particle. In some embodiments, the surface is directly coated with the capture reagent.

In embodiments, the capture reagent is an antibody, antigen, ligand, receptor, oligonucleotide, hapten, epitope, mimotope, or an aptamer. In embodiments of the methods of the invention, the capture reagent and the binding reagent are antibodies, or epitope binding portions thereof, capable of specifically binding a target molecule in or on the surface of the EV of interest.

In embodiments, the EV surface marker to which the capture reagent binds is common to EVs. Such surface markers include, but are not limited e.g., tetraspanins, such as CD9, CD37, CD63, CD81, CD82. In embodiments, the EV surface marker to which the capture reagent binds is specific to a tumor-derived EV. In embodiments, the EV surface marker to which the capture reagent binds is specific to a CRC-derived EV.

In embodiments, the EV surface marker to which the capture reagent binds is CD73, CD324, CD325, CD326, CD13, CD66a, CD10, CD31, CD36, CD141, CD14, CD54, CD26, MHC Class II molecules, FLT1.

In embodiments of the invention, the capture reagent is an antibody to a disease-specific target molecule in or on the surface of the EV. In embodiments, the EV surface marker to which the binding reagent binds is a cancer antigen. In embodiments, the cancer antigen to which the capture reagent and/or the binding reagent binds is CEA, CA19.9, CA50, CA125, CA15.3, mesothelin, cytokeratin-8, E-cadherin, EGFR, EpCAM, EphA2, NCAM, P-cadherin, cMET, Flt-3L, TNFR-2, cKit, ErbB2, FAP-a, or ANXA1.

In embodiments, the cancer antigen to which the capture reagent and/or the binding reagent binds is ABCB5, ASCT2, CA19-9, CCR7, CD10, CD105 (Endoglin), CD11b (ITGAM), CD13 (Aminopeptidase N), CD133 (PROM1), CD137, CD14, CD146 (MCAM), CD151, CD16, CD163, CD166 (ALCAM), CD171 (L1CAM), CD18, CD206, CD227 (MUC1), CD24, CD25, CD271, CD29, CD326, CD34, CD38, CD40, CD44, CD49e, CD49f, CD54 (ICAM-1), CD56 (NCAM1), CD66b (CEACAM8), CD66e (CEA), CD68, CD70, CD71, CD80, CD86, CD90, CD95, CD98, CL12A, Claudin-18.2, CXCR4, DLL3, DLL4, EGFR, Ephrin-B2, ERBB2 (HER2), FRα, GLUT1, GPC3, GPNMB (Osteoactivin), CEACAM6, HLA-DR, IL-13Ra2, ITGB3, LAT1, LGR5, LIV-1, LRC15, MCT1, MCT4, Mesothelin, MET, MMP14, Mucin-16 (CA125), CA 15-3, N-cadherin (CD325), Nectin-4, Notchl, Neuropilin-1, P-Cadherin, PDGFR-α/β, PSMA, ROR1, ROR2, TEM8, Tie-2, TLR4, TNFR1, TRAIL-R1, TRAIL-R2, uPAR, VCAM-1, VEGFR-1, VEGFR-2, VEGFR-3, xCT, CA9, CA12, TMPRSS4, Claudin-3, FGFR1, E-Cadherin (CD324), RANKL, EPHA2, Notch3, KIT, FGFR2, INSR, IGF1R, PSCA, IGF1R, PDGFRB, CRIPTO, BRAF, CD19, FGFR2, CCR2, CCR4, CCR5, TNK2, DDR1, LOXL2, or any combination thereof.

In embodiments, the EV comprises a surface marker of a surface-associated marker specific to immune cells, tumor immune micro-environment, tumor stroma to which the capture reagent and/or the binding reagent binds, such as: VTCN1, BST2, CTLA4, MRC1, DPP4, CD274, CD276, PDCD1, PECAM1, CD33, ENTPD1, CD47, NT5E, CLEC12A, CXCL10, FAP, FASLG, LGALS9, IL13RA2, ITGB6, LAG3, ROR1, SIGLEC10, SIGLEC15, SIGLEC7, SIGLEC9, HAVCR2, TACSTD2, C10orf54, CD4, CD2, CSF1R, IL6R, CD28, CD22, KLRK1, CD79B, CCR4, ICOS, MARCO, SIRPA, or any combination thereof.

In embodiments, the EV comprises a surface marker of a surface-associated marker specific or associated to platelets to which the capture reagent and/or the binding reagent binds, such as: PECAM1, CD36, CD40LG, ITGA2B, GP1BA, ITGB3, SELP, CD63, CLEC2A, GP6, TREM1, CD42a, CD42b, CD41, CD61, or any combination thereof.

In embodiments of the methods of the invention, the surface comprises an anchoring reagent. In the methods of the invention, the anchoring reagent is attached to the surface to allow linker oligonucleotide binding and/or amplicon binding in order to provide an additional indirect attachment point at the surface for the EV of interest. In embodiments, the anchoring reagent includes an oligonucleotide sequence, aptamer, aptamer ligand, antibody, antigen, ligand, receptor, hapten, epitope, or a mimotope; and optionally, the anchoring region can include an aptamer and the anchoring reagent can include an aptamer ligand/target molecule. The anchoring region, in embodiments, comprises a nucleic acid sequence and/or a DNA- or RNA-binding protein. The anchoring reagent comprises, in embodiments, oligonucleotide sequence and the anchoring reagent can include a complementary oligonucleotide sequence. The anchoring reagent, for example, can be a single stranded oligonucleotide sequence or a double stranded oligonucleotide sequence. In embodiments of the invention, the anchoring reagent features, etc., are disclosed in U.S. Pat. Nos. 10,408,823, 11,525,825, US Publication No. US 20220357318, and in International Appl. No. PCT/US2015/030925, published as WO 2015/175856, each of which is incorporated herein by reference it its entirety.

In additional embodiments, the amplicon is bound to the anchoring reagent at a position within 10 μm, 5 μm, or 100 nm of the location of the complex comprising the EV of interest on the surface.

Suitable surfaces for use in the methods of the present invention are known in the art, including conventional surfaces from the art of binding assays. Suitable surfaces are disclosed, for example, in U.S. Pat. Nos. 10,408,823, 11,525,825, US Publication No. US 20220357318, and in International Appl. No. PCT/US2015/030925, published as WO 2015/175856. Surfaces may be made from a variety of different materials including polymers (e.g., polystyrene and polypropylene), ceramics, glass, composite materials (e.g., carbon-polymer composites such as carbon-based inks). Suitable surfaces include the surfaces of macroscopic objects such as an interior surface of an assay container (e.g., test tubes, cuvettes, flow cells, FACS cell sorter, cartridges, wells in a multi-well plate, etc.), slides, assay chips (such as those used in gene or protein chip measurements), pins or probes, beads, filtration media, lateral flow media (for example, filtration membranes used in lateral flow test strips), etc.

Suitable surfaces also include particles (including but not limited to colloids or beads) commonly used in other types of particle-based assays e.g., magnetic, polypropylene, and latex particles, hydrogels, e.g. agarose, materials typically used in solid-phase synthesis e.g., polystyrene and polyacrylamide particles, and materials typically used in chromatographic applications e.g., silica, alumina, polyacrylamide, polystyrene. The materials may also be a fiber such as a carbon fibril. Microparticles may be inanimate or alternatively, may include animate biological entities such as cells, viruses, bacterium and the like. A particle used in the present method may be comprised of any material suitable for attachment to one or more capture or anchoring reagents, and that may be collected via, e.g., centrifugation, gravity, filtration or magnetic collection. A wide variety of different types of particles that may be attached to capture or anchoring reagents are sold commercially for use in binding assays. These include non-magnetic particles as well as particles comprising magnetizable materials which allow the particles to be collected with a magnetic field. In one embodiment, the particles are comprised of a conductive and/or semiconductive material, e.g., colloidal gold particles. The microparticles may have a wide variety of sizes and shapes. By way of example and not limitation, microparticles may be between 5 nanometers and 100 micrometers. Preferably microparticles have sizes between 20 nm and 10 micrometers. The particles may be spherical, oblong, rod-like, etc., or they may be irregular in shape.

The particles used in the present method may be coded to allow for the identification of specific particles or subpopulations of particles in a mixture of particles. The use of such coded particles has been used to enable multiplexing of assays employing particles as solid phase supports for binding assays. In one approach, particles are manufactured to include one or more fluorescent dyes and specific populations of particles are identified based on the intensity and/or relative intensity of fluorescence emissions at one or more wave lengths. This approach has been used in the Luminex xMAP systems (see, e.g., U.S. Pat. No. 6,939,720) and the Becton Dickinson Cytometric Bead Array systems. Alternatively, particles may be coded through differences in other physical properties such as size, shape, embedded optical patterns and the like. One or more particles provided in a mixture or set of particles may be coded to be distinguishable from other particles in the mixture by virtue of particle optical properties, size, shape, imbedded optical patterns and the like.

In a specific embodiment, the methods of the invention can be used in a multiplexed format by binding a plurality of different analytes to a plurality of capture reagents for those analytes, the capture analytes being immobilized on coded bead, such that the coding identifies the capture reagent (and analyte target) for a specific bead. The method may further comprise counting the number of beads that have a bound analyte (using the detection approaches described herein).

Alternatively or additionally, the capture reagents can be bound, directly or indirectly, to different discrete binding domains on one or more solid phases, e.g., as in a binding array wherein the binding domains are individual array elements, or in a set of beads wherein the binding domains are the individual beads, such that discrete assay signals are generated on and measured from each binding domain. If capture reagents for different analytes are immobilized in different binding domains, the different analytes bound to those domains can be measured independently. In one example of such an embodiment, the binding domains are prepared by immobilizing, on one or more surfaces, discrete domains of capture reagents that bind analytes of interest. Optionally, the surface(s) may define, in part, one or more boundaries of a container (e.g., a flow cell, well, cuvette, etc.) which holds the sample or through which the sample is passed. In a preferred embodiment, individual binding domains are formed on electrodes for use in electrochemical or electrochemiluminescence assays. Multiplexed measurement of analytes on a surface comprising a plurality of binding domains using electrochemiluminescence has been used in the Meso Scale Diagnostics, LLC, MULTI-ARRAY® and SECTOR® Imager line of products (see, e.g., U.S. Pat. Nos. 10,201,812, 7,842,246 and 6,977,722, the disclosures of which are incorporated herein by reference in their entireties).

Still further, the capture reagents can be bound, directly or indirectly, to an electrode surface, which optionally includes different discrete binding domains, as described above. The electrode surface can be a component of a multi-well plate and/or a flow cell. Electrodes can comprise a conductive material, e.g., a metal such as gold, silver, platinum, nickel, steel, iridium, copper, aluminum, a conductive alloy, or the like. They may also include oxide coated metals, e.g., aluminum oxide coated aluminum. The electrode can include a working and counter electrodes which can be made of the same or different materials, e.g., a metal counter electrode and carbon working electrode. In one specific embodiment, electrodes comprise carbon-based materials such as carbon, carbon black, graphitic carbon, carbon nanotubes, carbon fibrils, graphite, graphene, carbon fibers and mixtures thereof. In one embodiment, the electrodes comprise elemental carbon, e.g., graphitic, carbon black, carbon nanotubes, etc. Advantageously, they may include conducting carbon-polymer composites, conducting particles dispersed in a matrix (e.g. carbon inks, carbon pastes, metal inks, graphene inks), and/or conducting polymers. One specific embodiment of the invention is an assay module, preferably a multi-well plate, having electrodes (e.g., working and/or counter electrodes) that comprise carbon, e.g., carbon layers, and/or screen-printed layers of carbon inks.

In embodiments, the capture reagent is attached to the surface via a pair of short complementary oligonucleotides (one attached to the surface, the other attached to the capture reagent) that form stable duplexes in common biological buffers but can be denatured in a low salt buffer, and modestly elevated temperature is used to allow the capture reagent, e.g., antibody to be released. In embodiments, a restriction site in the complementary oligonucleotides that is cleaved by a restriction endonuclease is used. This has the advantage of being completely orthogonal to the denaturation that will be used, in embodiments, to purposely elute the stapled EVs, though a second restriction enzyme can also be used to elute the stapled EVs. Stapling sequence, diluents, and procedure are optimized to maximize retention of stapled EVs and minimize retention of non-stapled EVs. In embodiments, the captured EVs are co-labeled with STAG-labeled detection antibodies, and the ECL signal is compared with the ECL signal generated with and without elution, or with specific stapling and irrelevant stapling.

In embodiments, if the SMDA binds to the capture reagent, the first and second binding reagents, and if the anchoring reagent nucleotide sequence hybridizes to the second binding reagent nucleotide sequence, then a single-strand output oligonucleotide is generated that comprises the barcode oligonucleotide sequences of each of the anchoring reagent, and the first and second binding reagents. In embodiments, the anchoring reagent and the capture reagent are immobilized on a surface. In embodiments, the anchoring reagent and the capture reagent are immobilized on the same surface. In embodiments, the anchoring reagent and the capture reagent are linked together on a surface. In embodiments, the surface is biotinylated. In embodiments, the anchoring reagent comprises a labile linker, such as a restriction site or a UDG1 linkage sequence. In embodiments, the capture reagent comprises a labile linker, such as a restriction site or a UDG1 linkage sequence. In embodiments the anchoring reagent comprises an oligonucleotide. In embodiments the anchoring reagent comprises an antibody.

In embodiments, the third detection oligonucleotide comprises a third primer site. In embodiments, the method comprises extending the primer along the single-strand output oligonucleotide to form a double-strand output oligonucleotide. In embodiments, the method comprises amplifying the single-strand output oligonucleotide by strand-displacement amplification to form a double-strand output oligonucleotide; displacing a single-strand output oligonucleotide; further amplifying the output oligonucleotide by polymerase chain reaction (PCR); and sequencing the amplified output oligonucleotide to identify the barcode oligonucleotide sequences, thereby determining at least three unique surface markers of the SMDA.

In embodiments, the present disclosure provides a method of determining surface markers of a surface marker displaying agent (SMDA), comprising: (A) contacting a sample comprising an SMDA with: (a) a capture reagent that binds to a first surface marker of the SMDA; (b) an anchoring reagent comprising an anchoring oligonucleotide; (c) a first detection oligonucleotide comprising: (i) a first primer site; (ii) a first barcode sequence; (iii) a first blocker complement sequence; and (iv) a first hybridization sequence, wherein the first detection oligonucleotide and the anchoring oligonucleotide comprise complementary nucleotide sequences; (d) a first binding reagent that binds to a second surface marker of the SMDA, wherein the first binding reagent comprises a first conjugating oligonucleotide; (e) a second detection oligonucleotide comprising: (i) a second hybridization sequence; (ii) a third hybridization sequence comprising a second barcode sequence; and (iii) a fourth hybridization sequence; (f) a second binding reagent that binds to a third surface marker of the SMDA, wherein the second binding reagent comprises a second conjugating oligonucleotide; (g) a third detection oligonucleotide comprising: (i) a fifth hybridization sequence; (ii) a second blocker complement sequence; (iii) a third barcode sequence; (iv) a second primer site; and (iv) a third primer site; and (h) an oligonucleotide insert. In embodiments, the first hybridization sequence and the second hybridization sequence are complementary; the fourth hybridization sequence and the fifth hybridization sequence are complementary; and the third hybridization sequence is complementary to the oligonucleotide insert.

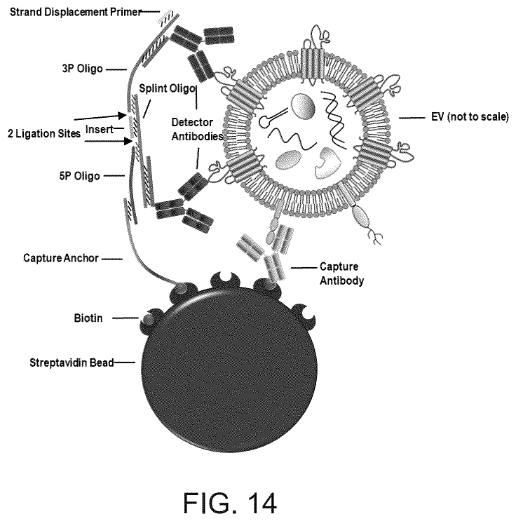

An illustration of this embodiment is in FIGS. 14 and 17. The method comprises (A) contacting a sample comprising an SMDA with: (a) a capture reagent that binds to a first surface marker of the SMDA; (b) an anchoring reagent comprising an anchoring oligonucleotide (“Biotin-Anchor” in FIG. 17); (c) a first detection oligonucleotide (“5′ Prime” in FIG. 17) comprising: (i) a first primer site (“FW Primer” in FIG. 17); (ii) a first barcode sequence (5′ “Barcode” in FIG. 17); (iii) a first blocker complement sequence (5′ “Blocker Stabilizer” in FIG. 17); and (iv) a first hybridization sequence (5′ “Splint Hyb Region” in FIG. 17), wherein the first detection oligonucleotide (“5′ Prime” in FIG. 17) and the anchoring oligonucleotide (“Biotin-Anchor” in FIG. 17) comprise complementary nucleotide sequences (“Anchor Region” in FIG. 17); (d) a first binding reagent that binds to a second surface marker of the SMDA, wherein the first binding reagent comprises a first conjugating oligonucleotide (“Antibody-Anchor Detector” in FIG. 17); (e) a second detection oligonucleotide (“Splint” in FIG. 17) comprising: (i) a second hybridization sequence; (ii) a third hybridization sequence comprising a second barcode sequence; and (iii) a fourth hybridization sequence; (f) a second binding reagent that binds to a third surface marker of the SMDA, wherein the second binding reagent comprises a second conjugating oligonucleotide (“Antibody-Anchor Detector” in FIG. 17); (g) a third detection oligonucleotide (“3′ Prime” in FIG. 17) comprising: (i) a fifth hybridization sequence (3′ “Splint Hyb Region” in FIG. 17); (ii) a second blocker complement sequence (3′ “Blocker stabilizer” in FIG. 17); (iii) a third barcode sequence (3′ “Barcode” in FIG. 17); (iv) a second primer site (“RV Primer Region” in FIG. 17); and (iv) a third primer site; and (h) an oligonucleotide insert (“Insert” in FIG. 17) comprising two common insert regions complementary to the second detection oligonucleotide (“Splint” in FIG. 17) and a reverse complement of the second barcode sequence (Insert “Barcode” in FIG. 17). In embodiments, the first hybridization sequence and the second hybridization hybridize to each other; the fourth hybridization sequence and the fifth hybridization sequence hybridize to each other; and the third hybridization sequence hybridizes to the oligonucleotide insert.

In embodiments, the method further comprises generating an output oligonucleotide by ligating the hybridized first detection oligonucleotide to the hybridized oligonucleotide insert, and ligating the hybridized oligonucleotide insert to the hybridized third detection oligonucleotide to form an output oligonucleotide, wherein the output oligonucleotide generated comprises the first barcode sequence, the second barcode sequence, and the third barcode sequence.

In embodiments, the method further comprises releasing and/or amplifying the output oligonucleotide; and sequencing the output oligonucleotide to identify the first, second, and third barcode sequences, thereby determining the surface markers of the SMDA.

In embodiments, the second detection oligonucleotide and the first conjugating oligonucleotide comprise complementary nucleotide sequences. In embodiments, the third detection oligonucleotide and the second conjugating oligonucleotide comprise complementary nucleotide sequences.

In embodiments, the first conjugating oligonucleotide and the second conjugating oligonucleotide are the same. In embodiments, the first conjugating oligonucleotide and the second conjugating oligonucleotide are different.

In embodiments, the first blocker complement sequence and at least a portion of the first hybridization sequence is hybridized to a first blocker oligonucleotide. In embodiments, the second blocker complement sequence and at least a portion of the fifth hybridization sequence is hybridized to a second blocker oligonucleotide. In embodiments, the first blocker oligonucleotide has a length of about 5 to about 20 nucleotides. In embodiments, the second blocker oligonucleotide has a length of about 5 to about 20 nucleotides. In embodiments, the first blocker complement sequence, and the second blocker complement sequence are the same. In embodiments, the first blocker complement sequence, and the second blocker complement sequence are different.

In embodiments, the capture reagent and/or the anchoring reagent are bound to a surface. In embodiments, the capture reagent is releasably bound to the surface. In embodiments, the anchoring reagent is releasably bound to the surface. In embodiments, the anchoring reagent and capture reagent are bound to the same surface. In embodiments, the capture reagent and/or anchoring reagent are releasably bound to the surface by a labile linker.

In embodiments, the SMDA is a cell, a virus or viral particle, an organelle, a vesicle, or combination thereof. In embodiments, the capture reagent, the first binding reagent, the second binding reagent, or a combination thereof, comprise an antibody or antigen binding fragment thereof. In embodiments, the capture reagent, the first binding reagent, the second binding reagent, or a combination thereof, binds to a surface marker common to EVs. In embodiments, the capture reagent, the first binding reagent, the second binding reagent, or a combination thereof binds to a cancer-related surface marker.

In embodiments, the disclosed method may be optimized through a series of modifications aimed at substantially shortening the sequences and oligonucleotides to maximize product formation and reduce non-specific signal

In embodiments, the first barcode sequence, the second barcode sequence, the third barcode sequence, or any combination thereof is about 5 to about 15 nucleotides long. In embodiments, the first detection oligonucleotide is about 20 to about 70 nucleotides long. In embodiments, the second detection oligonucleotide is about 40 to about 70 nucleotides long. In embodiments, the third detection oligonucleotide is about 40 to about 100 nucleotides long. In embodiments, the oligonucleotide insert is about 20 to about 40 nucleotides long.

In embodiments the anchoring oligonucleotide is complementary to the first primer site. In embodiments the anchoring oligonucleotide is attached directly or indirectly to a surface. In embodiments the anchoring oligonucleotide is attached to the surface via a biotin-streptavidin linkage. In embodiments, the second conjugating nucleotide is complementary to the second primer site. In embodiments, the first conjugation nucleotide is complementary to a 5′ region of the second detection oligonucleotide. In embodiments, the first conjugating oligonucleotide, the second conjugating oligonucleotide, or both are attached directly or indirectly to an antibody or antigen-binding fragment.

In some embodiments, the anchoring oligonucleotide is about 10 to about 70 nucleotides long. In some embodiments, the anchoring oligonucleotide is about 15 to about 30 nucleotides long. In some embodiments, the anchoring oligonucleotide is about 5 to about 25 nucleotides long. In some embodiments, the anchoring oligonucleotide is about 5, 10, 15, 20, 25, 30, 35, 40, 45, 50, 55, 60, 65, or 70 nucleotides long. In some embodiments, the anchoring oligonucleotide is about 40 nucleotides long.

In some embodiments, the first conjugating oligonucleotide is about 10 to about 70 nucleotides long. In some embodiments, the first conjugating oligonucleotide is about 15 to about 30 nucleotides long. In some embodiments, the first conjugating oligonucleotide is about 5 to about 25 nucleotides long. In some embodiments, the first conjugating oligonucleotide is about 5, 10, 15, 20, 25, 30, 35, 40, 45, 50, 55, 60, 65, or 70 nucleotides long. In some embodiments, the first conjugating oligonucleotide is about 20 nucleotides long.

In some embodiments, the second conjugating oligonucleotide is about 10 to about 70 nucleotides long. In some embodiments, the second conjugating oligonucleotide is about 15 to about 30 nucleotides long. In some embodiments, the second conjugating oligonucleotide is about 5 to about 25 nucleotides long. In some embodiments, the second conjugating oligonucleotide is about 5, 10, 15, 20, 25, 30, 35, 40, 45, 50, 55, 60, 65, or 70 nucleotides long. In some embodiments, the second conjugating oligonucleotide is about 20 nucleotides long.

In embodiments, the first barcode sequence is located between the first primer site and the blocker complement sequence. In embodiments, the second barcode sequence is located between the second and fourth hybridization sequence. In embodiments, the third barcode sequence is located between the second blocker complement sequence and the second primer site. In embodiments, the first, second, and/or third detection oligonucleotide may each comprise one or more barcode sequences.

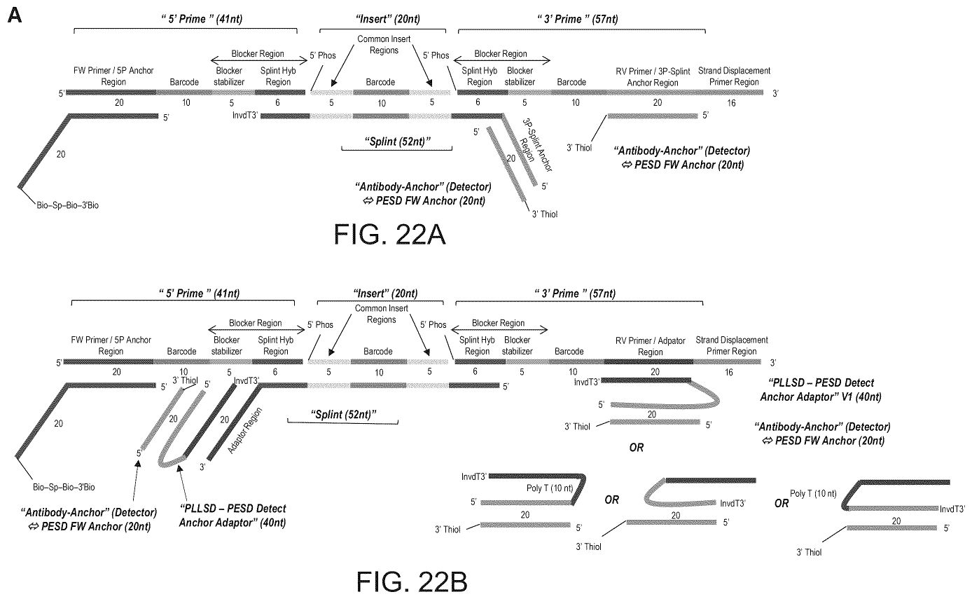

In embodiments, the PESD conjugating oligonucleotide (“forward anchor” in FIG. 20) can be used in lieu of the first and second conjugating oligonucleotides of the PLLSD method comprising an anchoring reagent as disclosed herein and exemplified in FIG. 22A. In this embodiment, the orientation of the second detection oligonucleotide is reversed for compatibility with the 3′thiol end of the PESD conjugating oligonucleotide.

In some embodiments, the present disclosure provides a PLLSD method or kit comprising at least one adaptor oligonucleotide. In embodiments, the adaptor oligonucleotide comprises an oligonucleotide sequence complementary to both the existing PLLSD first and second conjugating oligonucleotides and to the PESD conjugating oligonucleotide. This keeps the second detection oligonucleotide orientation unchanged and enables use of the PESD conjugating oligonucleotide, as exemplified in FIG. 22B.

In embodiments, the method comprises contacting the sample with a first adaptor oligonucleotide. In embodiments, the first adaptor oligonucleotide comprises: (i) a first sequence complementary to the first conjugating oligonucleotide, and (ii) a second sequence complementary to a 3′ sequence of the second detection oligonucleotide. In embodiments, the first and second sequence of the first adaptor oligonucleotide are directly linked to each other. In embodiments, the first and second sequence of the first adaptor oligonucleotide are linked to each other by a poly(T) sequence.

In embodiments the first adaptor oligonucleotide is about 40 nucleotides long. In embodiments the first adaptor oligonucleotide is about 20 to about 70 nucleotides long. In embodiments the first adaptor oligonucleotide is about 10 to about 50 nucleotides long. In embodiments the first adaptor oligonucleotide is about 10, 20, 30, 40, 50, 60, or 70 nucleotides long. In embodiments the first adaptor oligonucleotide is about 50 nucleotides long.

In embodiments, the first or second sequence is about 20, 25, or 30 nucleotides long. In embodiments, the first or second sequence is about 10 to about 40 nucleotides long. In embodiments, the first or second sequence is about 5 to about 25 nucleotides long. In embodiments, the poly(T) sequence is about 5 to about 15 nucleotides long. In embodiments, the poly(T) sequence is about 3, 4, 5, 6, 7, 8, 9, 10, 11, 12, 13, 14, or 15 nucleotides long.

In embodiments, the method further comprises contacting the sample with a second adaptor oligonucleotide. In embodiments, the second adaptor oligonucleotide comprises: (i) a first sequence complementary to the second conjugating oligonucleotide, and (ii) a second sequence complementary to the second primer site. In embodiments, the first and second sequence of the second adaptor oligonucleotide are directly linked to each other. In embodiments, the first and second sequence of the second adaptor oligonucleotide are linked to each other by a poly(T) sequence.

In embodiments the second adaptor oligonucleotide is about 40 nucleotides long. In embodiments the second adaptor oligonucleotide is about 20 to about 70 nucleotides long. In embodiments the second adaptor oligonucleotide is about 10 to about 50 nucleotides long. In embodiments the second adaptor oligonucleotide is about 10, 20, 30, 40, 50, 60, or 70 nucleotides long. In embodiments the second adaptor oligonucleotide is about 50 nucleotides long.

In embodiments, the first or second sequence is about 20, 25, or 30 nucleotides long. In embodiments, the first or second sequence is about 10 to about 40 nucleotides long. In embodiments, the first or second sequence is about 5 to about 25 nucleotides long. In embodiments, the poly(T) sequence is about 5 to about 15 nucleotides long. In embodiments, the poly(T) sequence is about 3, 4, 5, 6, 7, 8, 9, 10, 11, 12, 13, 14, or 15 nucleotides long.

### Constructs and Methods for Multimarker Isolation and Analysis of EVs

The present disclosure also provides a method of isolating multimarker EVs, comprising contacting a sample suspected of containing EVs with a first oligonucleotide-conjugated capture entity, a second oligonucleotide-conjugated splint entity, a third oligonucleotide-conjugated staple entity, and a surface.

In embodiments of the method, the capture entity, splint entity and staple entity are each independently selected from an antibody or antigen binding fragment thereof, antigen, ligand, receptor, oligonucleotide, hapten, epitope, mimitope, lipid binding protein, carbohydrate binding protein, DNA aptamer or RNA aptamer.

In embodiments, the capture entity, splint entity and staple entity are each an antibody or an antibody fragment.

In embodiments of the method, the first oligonucleotide-conjugated capture entity comprises a first oligonucleotide comprising a first Target nucleotide sequence, and a UDG1 labile linkage sequence. In embodiments, the capture entity is conjugated to the first oligonucleotide so that the first Target nucleotide sequence is located between the capture entity and the UDG1 labile linkage sequence.

In embodiments of the method, the second oligonucleotide-conjugated splint entity comprises a second oligonucleotide comprising a second Target nucleotide sequence, a restriction enzyme cleavage site and an additional nucleic acid sequence. In embodiments, the splint entity is conjugated to the second oligonucleotide so that the second Target nucleotide sequence is located between the splint entity and the restriction enzyme cleavage site and the additional nucleic acid sequence is located on the side of the second Target nucleotide sequence (either upstream or downstream) opposite the side that is conjugated to the splint entity.

In embodiments of the method, the third oligonucleotide-conjugated staple entity comprises a third oligonucleotide comprising a third Target nucleotide sequence and a restriction enzyme cleavage site. In embodiments, the staple entity is conjugated to the third oligonucleotide so that the third Target nucleotide sequence is located between the staple entity and the restriction enzyme cleavage site.

In embodiments of the method, complementary DNA sequences on the second oligonucleotide-conjugated splint entity and the third oligonucleotide-conjugated staple entity hybridize to form a double-stranded DNA restriction site. In embodiments, the surface has two capture oligonucleotides immobilized thereon. In embodiments, the first capture oligonucleotide comprises a sequence that is complementary to the additional nucleic acid sequence of the second oligonucleotide of the second oligonucleotide-conjugated splint entity and is capable of ligating to the end of the third oligonucleotide away from the third oligonucleotide-conjugated staple entity. In embodiments, the second capture oligonucleotide comprises a sequence that is complementary to the UDG1 labile linkage sequence of the first oligonucleotide on the first oligonucleotide-conjugated capture entity.

In embodiments of the method, the capture entity of the first oligonucleotide-conjugated capture entity is conjugated to the 5′ end of the first oligonucleotide. In embodiments, the splint entity of the second oligonucleotide-conjugated splint entity is conjugated to the 3′ end of the second oligonucleotide. In embodiments, the staple entity of the third oligonucleotide-conjugated staple entity is conjugated to the 5′ end of the third oligonucleotide.

In embodiments of the method, each oligonucleotide-conjugated entity is specific for a different EV surface marker. Exemplary EV surface markers are described. In embodiments of the method, at least one oligonucleotide-conjugated entity is specific for an EV surface marker that is associated with a disease or disorder.

In embodiments, the first oligonucleotide of the first oligonucleotide-conjugated capture entity comprises a 5′ first Target nucleotide sequence, and a UDG1 labile linkage sequence located 3′ of the first Target nucleotide sequence.

In embodiments, the third oligonucleotide of the third oligonucleotide-conjugated staple entity is conjugated to the staple entity at the 5′ end of the oligonucleotide and comprises a third Target nucleotide sequence and a restriction enzyme cleavage site positioned 3′ of the third Target nucleotide sequence.

In embodiments, the second oligonucleotide of the second oligonucleotide-conjugated splint entity is conjugated to the antibody at its 3′ end and comprises a second Target nucleotide sequence, a restriction enzyme cleavage site located on the 5′ side of the second Target nucleotide sequence, and an additional nucleic acid sequence positioned 5′ of the restriction enzyme cleavage site.

In embodiments, complementary DNA sequences on the second oligonucleotide-conjugated splint entity and the third oligonucleotide-conjugated staple entity hybridize to form a double-stranded DNA restriction site. In embodiments, the surface has two capture oligonucleotides immobilized thereon. In embodiments, the first capture oligonucleotide comprises a sequence that is complementary to the 5′-most portion of the oligonucleotide of the second oligonucleotide-conjugated splint entity and is capable of ligating at its 5′ end to the 3′ end of the oligonucleotide of the third oligonucleotide-conjugated staple entity. In embodiments, the capture oligonucleotide comprises a sequence that is complementary at its 3′ end to the UDG1 labile linkage sequence on the first oligonucleotide-conjugated capture entity.

In embodiments, the present disclosure provides a construct comprising: (a) a capture reagent, (b) at least three unique binding reagents and an oligonucleotide insert, and (c) at least two blocker oligonucleotides. In embodiments, each unique binding reagent comprises a detection sequence comprising a unique barcode oligonucleotide sequence.

In embodiments, the binding reagents of the construct comprise: a) a first binding reagent comprising a first detection sequence that comprises a (i) first hybridization sequence, wherein at least a portion of the first hybridization sequence is complexed with a first blocker oligonucleotide, (ii) a first primer site, and, optionally, (iii) a first connecting sequence hybridized to a conjugating oligonucleotide on the first binding reagent; b) a second binding reagent comprising a second detection sequence that comprises (i) a second hybridization sequence, (ii) a third hybridization sequence, (iii) a fourth hybridization sequence, and, optionally, (iv) a second connecting sequence hybridized to a second conjugating oligonucleotide on the second binding reagent; and c) a third binding reagent comprising a third detection sequence that comprises a fifth hybridization sequence, wherein at least a portion of the fifth hybridization sequence is complexed with a second blocker oligonucleotide, (ii) a second primer site, and, optionally, (iii) a third connecting sequence hybridized to a complementary sequence on the third binding reagent.

In embodiments, the first hybridization sequence and the second hybridization sequence are complementary. In embodiments, the fourth hybridization sequence and the fifth hybridization sequence are complementary. In embodiments, the third hybridization sequence is complementary to the oligonucleotide insert sequence. In embodiments the third binding reagent of the construct further comprises a third primer site.

In embodiments of the construct, the first detection sequence further comprises a first blocker complement sequence. In embodiments, the third detection sequence further comprises a second blocker complement sequence. In embodiments, the first blocker oligonucleotide is complementary to the first blocker complement sequence. In embodiments, the first blocker oligonucleotide is complementary to the first blocker complement sequence and the first hybridization sequence. In embodiments, the second blocker oligonucleotide is complementary to the second blocker complement sequence. In embodiments, the second blocker oligonucleotide is complementary to the second blocker complement sequence and the fifth hybridization sequence. In embodiments, the first blocker complement sequence and the second blocker complement sequence the same. In embodiments, nucleotide sequences of the first blocker complement sequence and the second blocker complement sequence are 100% identical.

In embodiments of the construct, the first, second and/or third binding reagents bind to any combination of surface markers selected from: CD9, CD73, CD324, CD325, CD326, CD13, CD66a, CD66e, CD10, CD31, CD36, CD141, CD14, CD54, CD26, MHC Class II molecules, and FLT1.

The present disclosure also provides a construct (a set of conjugates) for isolating multimarker EVs comprising: a first oligonucleotide-conjugated capture entity, a second oligonucleotide-conjugated splint entity, a third oligonucleotide-conjugated staple entity, and a surface, where each oligonucleotide-conjugated entity is termed a conjugate.

In embodiments of the construct, the first oligonucleotide-conjugated capture entity comprises a first oligonucleotide comprising a first Target nucleotide sequence, and a UDG1 labile linkage sequence. In embodiments, the capture entity is conjugated to the first oligonucleotide so that the first Target nucleotide sequence is located between the capture entity and the UDG1 labile linkage sequence.

In embodiments of the construct, the second oligonucleotide-conjugated splint entity comprises a second oligonucleotide comprising a second Target nucleotide sequence, a restriction enzyme cleavage site and an additional nucleic acid sequence. In embodiments, the splint entity is conjugated to the second oligonucleotide so that the second Target nucleotide sequence is located between the splint entity and the restriction enzyme cleavage site and the additional nucleic acid sequence is located on the side of the second Target nucleotide sequence (either upstream or downstream) opposite the side that is conjugated to the splint entity.

In embodiments of the construct, the third oligonucleotide-conjugated staple entity comprises a third oligonucleotide comprising a third Target nucleotide sequence and a restriction enzyme cleavage site. In embodiments, the staple entity is conjugated to the third oligonucleotide so that the third Target nucleotide sequence is located between the staple entity and the restriction enzyme cleavage site.

In embodiments of the construct, complementary DNA sequences on the second oligonucleotide-conjugated splint entity and the third oligonucleotide-conjugated staple entity hybridize to form a double-stranded DNA restriction site. In embodiments, the surface has two capture oligonucleotides immobilized thereon. In embodiments, the first capture oligonucleotide comprises a sequence that is complementary to the additional nucleic acid sequence of the second oligonucleotide of the second oligonucleotide-conjugated splint entity, and is capable of ligating to the end of the third oligonucleotide away from the third oligonucleotide-conjugated staple entity. In embodiments, the second capture oligonucleotide comprises a sequence that is complementary to the UDG1 labile linkage sequence of the first oligonucleotide on the first oligonucleotide-conjugated capture entity.

In embodiments of the construct, the capture entity of the first oligonucleotide-conjugated capture entity is conjugated to the 5′ end of the first oligonucleotide. In embodiments, the splint entity of the second oligonucleotide-conjugated splint entity is conjugated to the 3′ end of the second oligonucleotide. In embodiments, the staple entity of the third oligonucleotide-conjugated staple entity is conjugated to the 5′ end of the third oligonucleotide.

In embodiments of the construct, each oligonucleotide-conjugated entity is specific for a different EV surface marker. In embodiments, the first oligonucleotide of the first oligonucleotide-conjugated capture entity comprises a 5′ first Target nucleotide sequence, and a UDG1 labile linkage sequence located 3′ of the first Target nucleotide sequence.

In embodiments, the third oligonucleotide of the third oligonucleotide-conjugated staple entity is conjugated to the staple entity at the 5′ end of the oligonucleotide and comprises a third Target nucleotide sequence and a restriction enzyme cleavage site positioned 3′ of the third Target nucleotide sequence.

In embodiments, the second oligonucleotide of the second oligonucleotide-conjugated splint entity is conjugated to the splint entity at the 3′ end of the oligonucleotide and comprises a second Target nucleotide sequence, a restriction enzyme cleavage site located on the 5′ side of the second Target nucleotide sequence, and an additional nucleic acid sequence positioned 5′ of the restriction enzyme cleavage site.

In embodiments, complementary DNA sequences on the second oligonucleotide-conjugated splint entity and the third oligonucleotide-conjugated staple entity hybridize to form a double-stranded DNA restriction site. In embodiments, the surface has two capture oligonucleotides immobilized thereon. In embodiments, the first capture oligonucleotide comprises a sequence that is complementary to the 5′-most portion of the oligonucleotide of the second oligonucleotide-conjugated splint entity and is capable of ligating at its 5′ end to the 3′ end of the oligonucleotide of the third oligonucleotide-conjugated staple entity. In embodiments, the capture oligonucleotide comprises a sequence that is complementary at its 3′ end to the UDG1 labile linkage sequence on the first oligonucleotide-conjugated capture entity.

In embodiments of the method or construct, the first Target sequence, second Target sequence and third Target sequence have a length from about 15 to about 25 nucleotides. In embodiments, the first Target sequence, second Target sequence and third Target sequence have a length from about 20 to about 30 nucleotides. In embodiments, the first Target sequence, second Target sequence and third Target sequence have a length from about 17 to about 27 nucleotides. In embodiments, the first Target sequence, second Target sequence and third Target sequence have a length of about 15, 16, 17, 18, 19, 20, 21, 22, 23, 24, 25, 26, 27, 28, 29, 30, 31, 32, 33, 34 or 35 nucleotides.

In embodiments of the method or construct, the restriction enzyme cleavage site is a EcoRI, EcoRII, BamHI, HindIII, TaqI, NotI, HinFI, Sau3AI, PvuII, SmaI, HaeIII, HgaI, AluI, EcoRV, EcoP15I, KpnI, PstI, SacI, SalI, ScaI, SpeI, SphI, StuI or XbaI cleavage site.

In embodiments, the UDG1 labile linkage sequence is cleaved by uracil-DNA glycosylase (UDG). UDG removes uracil from DNA. In embodiments, when UDG removes uracil from a segment of DNA, the segment is no longer able to hybridize with its second strand, causing denaturation. In embodiments the UDG1 labile linkage sequence is a DNA sequence comprising uracil nucleotides.

In embodiments of the method or construct, the first oligonucleotide-conjugated capture entity binds to a surface marker common to EVs. Surface markers common to EVs are described herein. In embodiments, the marker is a tetraspanin. In embodiments, the tetraspanin is CD9, CD63, or CD81. In embodiments, the first oligonucleotide-conjugated capture entity binds to a surface marker that is not common to EVs. In embodiments, the first oligonucleotide-conjugated antibody binds to a surface marker selected from CD2, CD3, CD4, CD5, CD8, CD10 (NEP), CD11b (ITGAM), CD13(AAP), CD14, CD15 (SSEA-1), CD16 (FcγRIII), CD18 (ITGB2), CD25(IL-2Ra), CD26(DPPIV), CD28, CD29 (ITGB1), CD31 (PECAM-1), CD32b (FcγRII), CD33 (Siglec-3), CD36 (GPIV), CD38, CD40, CD41 (GP2B), CD42b(GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM1), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166(ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1(VEGFR1) and GLUT1 (SLC2A1).

In embodiments of the method of isolating EVs, the Target nucleotide sequences remain intact following EV release and can be hybridized to complementary dye-conjugated in situ probes (complementary to the Target sequence). In embodiments, the in-situ probes allow the EV to be fluorescently labeled for multi-color co-localization microscopy/FISH microscopy. In embodiments, a different fluorescent label is attached to each probe directed to a different Target sequence. In embodiments, the probe complementary to the probe complementary to the first Target nucleotide sequence, the probe complementary to the second Target nucleotide sequence and the probe complementary to the third Target nucleotide sequence each have a different fluorescent label. In other embodiments, one or more of the Target sequences can be hybridized to a complementary probe that is biotinylated for subsequent EV pulldown or recapture onto a streptavidin surface for additional analyses including ECL assays.

In embodiments, the multimarker EV isolation method and construct described in this section is used in a method of isolating or detecting an EV requiring a stapling step. Such methods are described below and in and in US Patent Publication 2021/0382043, which is incorporated by reference in its entirety herein. In embodiments, the first oligonucleotide-conjugated capture reagent is used in the isolation of an EV but not used in the detection of an EV.

As used herein, the term “isolating” an EV of interest means to have no more than 5% by weight of any other non-EV components (i.e., unwanted components), and preferably no more than 4%, 3%, 2% or 1% by weight of unwanted components, or preferably no more than 0.8%, 0.6%, 0.4%, 0.2% or 0.1% or less by weight of the unwanted component. The term “isolating” also encompasses amounts of unwanted components that are undetectable by current methods for detecting such components. As used herein, the term “isolating” is synonymous with enriching and purifying.

### Reagents and Entities

The terms capture reagent and binding reagent are generally used herein in the context of methods and kits for detecting EVs, while the terms capture entity, splint entity and staple entity are generally used herein in the context of methods and kits for isolating EVs. In embodiments, these terms are used interchangeably to describe a structure that binds to a target on the surface of an EV. As a specific, non-limiting example, in the methods and kits herein, each of the capture reagent, first binding reagent, second binding reagent, third binding reagent, capture entity, splint entity and staple entity can be an antibody or an antigen binding fragment of an antibody.

In embodiments of the method, the capture reagent, first binding reagent, second binding reagent, capture entity, splint entity and staple entity can be an antibody or antigen binding fragment thereof, antigen, ligand, receptor, oligonucleotide, hapten, epitope, mimitope, lipid binding protein, carbohydrate binding protein, DNA aptamer or RNA aptamer. In embodiments, the entity is a lipid binding protein selected from TIM-4 (T cell immunoglobulin mucin protein 4) and TIM-1 (T cell immunoglobulin mucin protein 1). In embodiments, the entity is a lipid binding protein selected from one of the fatty-acid binding proteins (FABPs): FABP 1, FABP 2, FABP 3, FABP 4, FABP 5, FABP 6, FABP 7, FABP 8, FABP 9, FABP 10, FABP 11, or FABP 12. In embodiments, the entity is a carbohydrate binding protein which is a lectin. In embodiments, the lectin is selected from ConA, LCH, GNA, RCA, PNA, AIL, VVL, WGA, SNA, MAL, MAH, UEA, or AAL. In embodiments, the entity is DNA aptamer or RNA aptamer designed to bind a cell surface marker on the surface of the EV. In embodiments, one or more of the capture entity, splint entity and staple entity are of a different type of entity than the other entities.

In embodiments of the method, a wash buffer is added during or after any one or more steps provided herein. In embodiments, the wash buffer comprises arginine. In embodiments, the wash buffer comprises arginine at a concentration of at least 100 mM. In embodiments, the wash buffer comprises arginine at about 100 mM to about 1000 mM, at about 200 mM to about 800 mM, at about 300 mM to about 600 mM, at about 400 mM to about 500 mM, at about 350 mM to about 750 mM, at about 400 mM to about 600 mM or at about 500 mM.

### Assaying the EV

In embodiments of the disclosure, the EV of interest is assayed. In embodiments, the assay is an ultrasensitive assay. In embodiments of the disclosure, the EV of interest is assayed while bound to the surface, either by both attachment points, e.g., by the capture reagent and by the binding reagent/anchoring reagent, or after the capture reagent is released from the surface. In embodiments, the assaying comprises contacting a detectably labeled oligonucleotide with the surface, wherein the oligonucleotide is complementary to the amplicon. In embodiments, the binding reagent is detectably labeled.

In embodiments of the disclosure, bound EVs of interest are subjected to a measuring step, which are known to those of skill in the art, for example, as disclosed in U.S. Pat. Nos. 10,408,823, 11,525,825, US Publication No. US 20220357318, and in International Appl. No. PCT/US2015/030925, published as WO 2015/175856, all of which are incorporated by reference in their entirety. In embodiments, EVs of interest are measured using an ultrasensitive assay format for soluble proteins that marries a variation of proximity ligation amplification (PLA) with ECL detection to provide state-of-the-art sensitivity. The measuring step of the method can comprise imaging an optical signal from the surface to generate an image that consists of a plurality of pixels, wherein each resolvable binding region maps to one or more pixels or groups of pixels in the image. Image analysis to identify pixels or sets of pixels having a signal indicative of a binding event (detection complex) can be accomplished using art recognized methods.

In one embodiment, the resolvable binding regions are elements of an array. In embodiments, the array is an array of micro-wells or nanowells, e.g., individual depressions or wells of a unitary substrate. Preferably, the volume of the wells is less than 500 μL, 300 μL, 150 μL, 100 μL, 10 μL, 1 μL, 100 nL, preferably less than 50 nL. In one embodiment, the volume of the wells ranges from approximately 10 aL-100 pL. Optionally, the wells may be configured to hold a microparticle.

In one embodiment, at least 50% of the resolvable binding regions positioned on a substrate and addressed during an assay contain either zero or one analyte molecule. Preferably, at least 80%, more preferably at least 95%, and most preferably at least 99% of the resolvable binding regions contain either zero or one analyte molecule. The concentration of analyte molecules in the sample is determined at least in part using a calibration curve, a Poisson distribution analysis and/or a Gaussian distribution analysis of the number of binding regions that contain at least one or one analyte molecule. In a specific embodiment, the surface comprises a plurality of particles each including a plurality of capture reagents for an analyte molecule and the plurality of particles is distributed across a plurality of resolvable binding regions (e.g., an array of micro- or nano-wells). Therefore, the method includes: (i) binding one or more analyte molecules to one or more capture reagents on the surface, (ii) distributing the plurality of particles across an array of resolvable binding regions; and (iii) determining the presence or absence of an analyte molecule in each resolvable binding regions, so as to identify the number of binding domains that contain an analyte molecule and/or the number of binding domains that do not contain an analyte molecule.

Alternatively, labels used to detect analyte molecules can be fluorescent species that can be used in single molecule fluorescence detection, e.g., fluorescence correlation spectroscopy, and/or fluorescence cross-correlation spectroscopy. Single molecule fluorescence detection comprises flowing an eluent that includes a detectable species through a capillary, focusing a light source on a volume within the capillary to create an interrogation zone and observing the interrogation zone with a light detector to detect the passage of fluorescent molecules through the interrogation zone.

In one embodiment, the EV of interest in the sample may be measured using electrochemiluminescence-based assay formats, e.g., electrochemiluminescence (ECL) based immunoassays. Species that can be induced to emit ECL (ECL-active species) have been used as ECL labels, e.g., i) organometallic compounds where the metal is from, for example, the noble metals of group VIII, including Ru-containing and Os-containing organometallic compounds such as the tris-bipyridyl-ruthenium (RuBpy) moiety and ii) luminol and related compounds. Species that participate with the ECL label in the ECL process are referred to herein as ECL coreactants. Commonly used coreactants include tertiary amines (e.g., see U.S. Pat. No. 5,846,485), oxalate, and persulfate for ECL from RuBpy and hydrogen peroxide for ECL from luminol (see, e.g., U.S. Pat. No. 5,240,863). The light generated by ECL labels can be used as a reporter signal in diagnostic procedures (Bard et al., U.S. Pat. No. 5,238,808, herein incorporated by reference). For instance, an ECL label can be covalently coupled to a binding agent such as an antibody, nucleic acid probe, receptor or ligand; the participation of the binding reagent in a binding interaction can be monitored by measuring ECL emitted from the ECL label. Alternatively, the ECL signal from an ECL-active compound may be indicative of the chemical environment (see, e.g., U.S. Pat. No. 5,641,623 which describes ECL assays that monitor the formation or destruction of ECL coreactants).

The methods of the disclosure may be applied to singleplex or multiplex formats where multiple assay measurements are performed on a single sample. Multiplex measurements that can be used with the disclosure include, but are not limited to, multiplex measurements i) that involve the use of multiple sensors; ii) that use discrete assay domains on a surface (e.g., an array) that are distinguishable based on location on the surface; iii) that involve the use of reagents coated on particles that are distinguishable based on a particle property such as size, shape, color, etc.; iv) that produce assay signals that are distinguishable based on optical properties (e.g., absorbance or emission spectrum) or v) that are based on temporal properties of assay signal (e.g., time, frequency or phase of a signal).

The disclosure includes methods for detecting and counting individual detection complexes. In a specific embodiment, the surface can comprise a plurality of capture reagents for one or more EVs that are present in a sample and the plurality of capture reagents are distributed across a plurality of resolvable binding regions positioned on the surface. Under the conditions used to carry out and analyze a measurement, a “resolvable binding region” is the minimal surface area associated with an individual binding event that can be resolved and differentiated from another area in which an additional individual binding event is occurring. Therefore, the method consists of binding one or more EVs of interest to one or more capture reagents on the surface, determining the presence or absence of the EV in a plurality of resolvable binding regions on the surface, and identifying the number of resolvable binding regions that contain an EV of interest and/or the number of analyte domains that do not contain an EV of interest.

The resolvable binding regions can be optically interrogated, in whole or in part, i.e., each individual resolvable binding region can be individually optically interrogated and/or the entire surface comprising a plurality of resolvable binding regions can be imaged and one or more pixels or groupings of pixels within that image can be mapped to an individual resolvable binding region. A resolvable binding region may also be a microparticle within a plurality of microparticles. The resolvable binding regions exhibiting changes in their optical signature can be identified by a conventional optical detection system. Depending on the detected species (e.g., type of fluorescence entity, etc.) and the operative wavelengths, optical filters designed for a particular wavelength can be employed for optical interrogation of the resolvable binding regions. In embodiments where optical interrogation is used, the system can comprise more than one light source and/or a plurality of filters to adjust the wavelength and/or intensity of the light source. In some embodiments, the optical signal from a plurality of resolvable binding regions is captured using a CCD camera. Other non-limiting examples of camera imaging systems that can be used to capture images include charge injection devices (CIDs), complementary metal oxide semiconductors (CMOSs) devices, scientific CMOS (sCMOS) devices, and time delay integration (TDI) devices, as will be known to those of ordinary skill in the art. In some embodiments, a scanning mirror system coupled with a photodiode or photomultiplier tube (PMT) can be used for imaging.

Additional methods of interrogating whole EVs are known in the art, such as by bioluminescence, and nuclear magnetic resonance (NMR). In additional embodiments, for example, the EV of interest is assessed by quantitative polymerase chain reaction, next generation sequencing, or both.

### Controls

In an additional embodiment, the assay formats described herein further include one or more control assays. A negative control can be included on a binding domain which includes a capture reagent or capture entity that does not have a corresponding detection antibody, thereby providing a consistent background signal for all samples. Measurement of signal above a preset threshold value can indicate improper assay processing or the presence of a sample-dependent matrix effect causing non-specific binding of labeled detection probe. Moreover, a specimen control can also be included in the assay for a human target antigen (such as a secreted or intracellular protein) that performs multiple control functions. A positive signal will indicate the presence of human material, and therefore test for sample addition and quality. Measurement of a signal below a predefined threshold would indicate that no sample was added, that a failure in the reagents or process occurred, or that substances that interfere with amplification or detection are present. In addition to internal controls, external positive and negative controls can also be used with the method and/or kit. The negative control comprises a representative matrix without any target proteins.

In embodiments, a control EV is used to establish the performance of the assay or provide a reliable sample for normalizing data, or both. In embodiments, control EVs facilitate comparison of results between plates or experiments, or both. In embodiments, a control EV is used for correction of nonlinearity of an assay at upper and lower ends of the calibration curve. For example, it may be advantageous to utilize a synthetic EV, which allows for selection of surface antigens, and the copy number can be tuned to match the biological material of interest. In embodiments, the control EV has similar size and density to the EV of interest. In embodiments, the synthetic EV is produced using polymer beads of similar size and density to small EVs. In embodiments, tetraspanin proteins are attached to the surface of the synthetic EV.

In embodiments, well-characterized, biologically-derived EVs are used as controls. In embodiments, control EVs are produced from a cell line selected for its efficiency at producing EVs. In embodiments, EVs from cell lines or biofluids are used as negative controls, such as from platelets, PBMCs, THP-1 cells, Expi293 cells, and HCT-15 cells. In embodiments, synthetic EVs, such as unilamellar vesicles or beads that have similar physiochemical properties as the EVs of interest, are used as controls.

### Kits

The disclosure further provides a kit for determining surface markers of an SMDA, for identifying SMDAs that harbor combinations of surface markers, for detecting populations of SMDAs having certain surface markers, and/or for detecting or quantifying multiple populations of SMDAs where each population has a specific set of surface markers.

In embodiments, the kit comprises, in one or more vials, containers, or compartments: a capture reagent, at least three unique binding reagents and an oligonucleotide insert, and at least two blocker oligonucleotides.

In embodiments, each unique binding reagent comprises a detection sequence comprising a unique barcode oligonucleotide sequence. In embodiments, the plurality of binding reagents comprises: a) a first binding reagent comprising a first detection sequence that comprises a (i) first hybridization sequence, wherein at least a portion of the first hybridization sequence is complexed with a first blocker oligonucleotide, (ii) a first primer site, and (iii) a first connecting sequence hybridized to a complementary sequence on the first binding reagent; b) a second binding reagent comprising a second detection sequence that comprises (i) a second hybridization sequence, (ii) a third hybridization sequence, and (iii) a fourth hybridization sequence; and c) a third binding reagent comprising a third detection sequence that comprises a fifth hybridization sequence, wherein at least a portion of the fifth hybridization sequence is complexed with a second blocker oligonucleotide, (ii) a second primer site, and (iii) a third connecting sequence hybridized to a complementary sequence on the third binding reagent.

In embodiments, the first hybridization sequence and the second hybridization sequence are complementary. In embodiments, the fourth hybridization sequence and the fifth hybridization sequence are complementary. In embodiments, the third hybridization sequence is complementary to the oligonucleotide insert sequence. In embodiments, the third binding reagent further comprises a third primer site. In embodiments, the first detection sequence comprises a first blocker complement sequence. In embodiments, the first blocker oligonucleotide is complementary to the first blocker complement sequence. In embodiments, the first blocker oligonucleotide is complementary to the first hybridization sequence and the first blocker complement sequence. In embodiments, the third detection sequence comprises a second blocker complement sequence. In embodiments, the second blocker oligonucleotide is complementary to the second blocker complement sequence. In embodiments, the second blocker oligonucleotide is complementary to the fifth hybridization sequence and the second blocker complement sequence.

In embodiments of the kit, the first binding reagent further comprises a first conjugating oligonucleotide and the first detection sequence comprises a first connecting sequence complementary to the first conjugating oligonucleotide. In embodiments, the second binding reagent further comprises a second conjugating oligonucleotide and the second detection sequence comprises a second connecting sequence complementary to the second conjugating oligonucleotide. In embodiments, the third binding reagent further comprises a third conjugating oligonucleotide and the third detection sequence comprises a third connecting sequence complementary to the third conjugating oligonucleotide. In embodiments, the first, second, and third connecting sequences are unique. In embodiments, the first, second, and third connecting sequences are the same. In embodiments, the first, second, and third connecting sequences are of the same length. In embodiments, the first, second, and third connecting sequences are about 5 to about 50 nucleotides long. In embodiments, the first, second, and third connecting sequences comprise tandem repeats.

In embodiments, the kit comprises a hairpin blocker to inhibit inter-EV ligation. In embodiments, the kit further comprises a strand-displacement primer and strand-displacement polymerase. In embodiments, the strand-displacement polymerase comprises a Klenow fragment.

The disclosure further provides a kit for detecting an EV in a sample comprising, in one or more vials, containers, or compartments: (i) a capture reagent; (ii) a first binding reagent that binds a first surface marker of the EV, wherein the first binding reagent comprises a first detection sequence that comprises a first hybridization sequence, and a first primer site; (iii) a second binding reagent that binds a second surface marker of the EV, wherein the second binding reagent comprises a second detection sequence that comprises a second hybridization sequence, a third hybridization sequence, and a fourth hybridization sequence; (iv) a third binding reagent comprising a third detection sequence, wherein the third detection sequence comprises a fifth hybridization sequence, and a second primer site; and (v) an oligonucleotide insert comprising an oligonucleotide insert sequence. In embodiments, at least a portion of the first detection sequence is hybridized with a first blocker oligonucleotide. In embodiments, at least a portion of the third detection sequence is hybridized with a second blocker oligonucleotide.

In embodiments, the third binding reagent, and/or third detection sequence, and the capture reagent are bound to the surface. In embodiments, the third detection sequence is releasably bound to the surface. In embodiments, the third binding reagent is releasably bound to the surface. In embodiments, the kit may further comprise a capture reagent releasably bound to the surface. In embodiments, the third binding reagent, and/or third detection sequence, and the capture reagent are independently bound to the surface. In embodiments, the third binding reagent, and/or third detection sequence, and the capture reagent are bound to the same surface. In embodiments, the third binding reagent, and/or third detection sequence, and the capture reagent are releasably bound to the same surface. In embodiments, the third binding reagent, and/or third detection sequence, and the capture reagent are bound to separate surfaces. In embodiments, the third binding reagent, and/or third detection sequence, and the capture reagent are releasably bound to separate surfaces.

In embodiments of the kit, the first hybridization sequence and the second hybridization sequence are complementary. In embodiments, the fourth hybridization sequence and the fifth hybridization sequence are complementary. In embodiments, the third hybridization sequence is complementary to the oligonucleotide insert sequence.

In embodiments of the kit, the capture reagent is bound to a surface. In embodiments, the third binding reagent is bound to the same surface as the capture reagent. In embodiments, the third binding reagent binds a third surface marker of the EV.

In embodiments, the kit further comprises a first primer complementary to the first primer site and a second primer complementary to the second primer site. In embodiments, the kit comprises multiple different capture reagents and multiple different first, second, and third binding reagents to allow combinatorial analysis of markers.

In embodiments of the kit, the capture reagent binds to a surface marker common to EVs. In embodiments, the marker is a tetraspanin. In embodiments, the tetraspanin is CD9, CD63, or CD81. In embodiments, the capture reagent binds to a surface marker that is not common to EVs. In embodiments, the capture reagent binds to a surface marker selected from CD2, CD3, CD4, CD5, CD8, CD10 (NEP), CD11b (ITGAM), CD13(AAP), CD14, CD15 (SSEA-1), CD16 (FcγRIII), CD18 (ITGB2), CD25(IL-2Ra), CD26(DPPIV), CD28, CD29 (ITGB1), CD31 (PECAM-1), CD32b (FcγRII), CD33 (Siglec-3), CD36 (GPIV), CD38, CD40, CD41 (GP2B), CD42b(GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM1), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166(ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1(VEGFR1) and GLUT1 (SLC2A1).

The disclosure further provides a kit for detecting an EV in a sample comprising, in one or more vials, containers, or compartments at least three unique binding reagents and an oligonucleotide insert.

In embodiments of the kit, each unique binding reagent comprises a detection sequence comprising a unique barcode oligonucleotide sequence. In embodiments, when at least three unique binding reagents bind to three unique surface markers of the SMDA, an output oligonucleotide is generated that comprises the barcode oligonucleotide sequences of each of the three unique binding reagents. In embodiments, the output oligonucleotide is capable of being sequenced to identify the three unique surface markers of the SMDA.

In embodiments, the plurality of binding reagents comprises: a. a first binding reagent comprising a first detection sequence that comprises a first hybridization sequence, and a first primer site; b. a second binding reagent comprising a second detection sequence that comprises a second hybridization sequence, a third hybridization sequence, and a fourth hybridization sequence; and c. a third binding reagent comprising a third detection sequence that comprises a fifth hybridization sequence, and a second primer site, wherein the first hybridization sequence and the second hybridization sequence are complementary.

In embodiments, the fourth hybridization sequence and the fifth hybridization sequence are complementary. In embodiments, the third hybridization sequence is complementary to the oligonucleotide insert sequence.

In embodiments of the kit, the first binding reagent binds to a surface marker common to EVs. In embodiments, the marker is a tetraspanin. In embodiments, the tetraspanin is CD9, CD63, or CD81. In embodiments, the first binding reagent binds to a surface marker that is not common to EVs. In embodiments, the first binding reagent binds to a surface marker selected from CD2, CD3, CD4, CD5, CD8, CD10 (NEP), CD11b (ITGAM), CD13(AAP), CD14, CD15 (SSEA-1), CD16 (FcγRIII), CD18 (ITGB2), CD25(IL-2Ra), CD26(DPPIV), CD28, CD29 (ITGB1), CD31 (PECAM-1), CD32b (FcγRII), CD33 (Siglec-3), CD36 (GPIV), CD38, CD40, CD41 (GP2B), CD42b(GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM1), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166(ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1(VEGFR1) and GLUT1 (SLC2A1).

In embodiments of the kit, the first, second and/or third binding reagents bind to any combination of surface markers selected from: CD9, CD73, CD324, CD325, CD326, CD13, CD66a, CD66e, CD10, CD31, CD36, CD141, CD14, CD54, CD26, MHC Class II molecules, and FLT1.

In embodiment of the kit, the first, second and/or third binding reagents bind to any combination of surface markers selected from: CD12, ABCB5, ASCT2, CA19-9, CCR7, CD10, CD105 (Endoglin), CD11b (ITGAM), CD13 (Aminopeptidase N), CD133 (PROM1), CD137, CD14, CD146 (MCAM), CD151, CD16, CD163, CD166 (ALCAM), CD171 (L1CAM), CD18, CD206, CD227 (MUC1), CD24, CD25, CD271, CD29, CD326, CD34, CD38, CD40, CD44, CD49e, CD49f, CD54 (ICAM-1), CD56 (NCAM1), CD66b (CEACAM8), CD66e (CEA), CD68, CD70, CD71, CD80, CD86, CD90, CD95, CD98, CL12A, Claudin-18.2, CXCR4, DLL3, DLL4, EGFR, Ephrin-B2, ERBB2 (HER2), FRα, GLUT1, GPC3, GPNMB (Osteoactivin), CEACAM6, HLA-DR, IL-13Rα2, ITGB3, LAT1, LGR5, LIV-1, LRC15, MCT1, MCT4, Mesothelin, MET, MMP14, Mucin-16 (CA125), CA 15-3, N-cadherin (CD325), Nectin-4, Notchl, Neuropilin-1, P-Cadherin, PDGFR-α/β, PSMA, ROR1, ROR2, TEM8, Tie-2, TLR4, TNFR1, TRAIL-R1, TRAIL-R2, uPAR, VCAM-1, VEGFR-1, VEGFR-2, VEGFR-3, xCT, CA9, CA12, TMPRSS4, Claudin-3, FGFR1, E-Cadherin (CD324), RANKL, EPHA2, Notch3, KIT, FGFR2, INSR, IGF1R, PSCA, IGF1R, PDGFRB, CRIPTO, BRAF, CD19, FGFR2, CCR2, CCR4, CCR5, TNK2, DDR1, LOXL2.

In embodiment of the kit, the first, second and/or third binding reagents bind to any combination of surface markers selected from: VTCN1, BST2, CTLA4, MRC1, DPP4, CD274, CD276, PDCD1, PECAM1, CD33, ENTPD1, CD47, NT5E, CLEC12A, CXCL10, FAP, FASLG, LGALS9, IL13RA2, ITGB6, LAG3, ROR1, SIGLEC10, SIGLEC15, SIGLEC7, SIGLEC9, HAVCR2, TACSTD2, C10orf54, CD4, CD2, CSF1R, IL6R, CD28, CD22, KLRK1, CD79B, CCR4, ICOS, MARCO, SIRPA.

In embodiment of the kit, the first, second and/or third binding reagents bind to any combination of surface markers selected from: PECAM1, CD36, CD40LG, ITGA2B, GP1BA, ITGB3, SELP, CD63, CLEC2A, GP6, TREM1, CD42a, CD42b, CD41, CD61.

In embodiments of any of the above kits, the kit further comprises a ligase.

The disclosure further provides a kit for isolating multimarker EVs comprising, in one or more vials, containers, or compartments: a first oligonucleotide-conjugated capture entity, a second oligonucleotide-conjugated splint entity, a third oligonucleotide-conjugated staple entity, and a surface.

In embodiments of the kit, the capture entity, splint entity and staple entity are each independently selected from an antibody or antigen binding fragment thereof, antigen, ligand, receptor, oligonucleotide, hapten, epitope, mimitope, lipid binding protein, carbohydrate binding protein, DNA aptamer or RNA aptamer. In embodiments, one or more of the capture entity, splint entity and staple entity are of a different type of entity than the other entities.

In embodiments of the kit, the capture entity, splint entity and staple entity are each an antibody or an antibody fragment.

In embodiments of the kit, the first oligonucleotide-conjugated capture entity comprises a first oligonucleotide comprising a first Target nucleotide sequence, and a UDG1 labile linkage sequence. In embodiments, the capture entity is conjugated to the first oligonucleotide so that the first Target nucleotide sequence is located between the capture entity and the UDG1 labile linkage sequence.

In embodiments of the kit, the second oligonucleotide-conjugated splint entity comprises a second oligonucleotide comprising a second Target nucleotide sequence, a restriction enzyme cleavage site and an additional nucleic acid sequence. In embodiments, the splint entity is conjugated to the second oligonucleotide so that the second Target nucleotide sequence is located between the splint entity and the restriction enzyme cleavage site, and the additional nucleic acid sequence is located on the side of the second Target sequence (either upstream or downstream) opposite the side that is conjugated to the splint entity.

In embodiments of the kit, the third oligonucleotide-conjugated staple entity comprises a third oligonucleotide comprising a third Target nucleotide sequence and a restriction enzyme cleavage site. In embodiments, the staple entity is conjugated to the third oligonucleotide so that the third Target nucleotide sequence is located between the staple entity and the restriction enzyme cleavage site.

In embodiments of the kit, complementary DNA sequences on the second oligonucleotide-conjugated splint entity and the third oligonucleotide-conjugated staple entity hybridize to form a double-stranded DNA restriction site. In embodiments, the surface has two capture oligonucleotides immobilized thereon. In embodiments, the first capture oligonucleotide comprises a sequence that is complementary to the additional nucleic acid sequence of the second oligonucleotide of the second oligonucleotide-conjugated splint entity and is capable of ligating to the end of the third oligonucleotide away from the third oligonucleotide-conjugated staple entity. In embodiments, the second capture oligonucleotide comprises a sequence that is complementary to the UDG1 labile linkage sequence of the first oligonucleotide on the first oligonucleotide-conjugated capture entity.

In embodiments of the kit, the capture entity of the first oligonucleotide-conjugated capture entity is conjugated to the 5′ end of the first oligonucleotide. In embodiments, the splint entity of the second oligonucleotide-conjugated splint entity is conjugated to the 3′ end of the second oligonucleotide. In embodiments, the staple entity of the third oligonucleotide-conjugated staple entity is conjugated to the 5′ end of the third oligonucleotide.

In embodiments, the third oligonucleotide of the third oligonucleotide-conjugated staple entity is conjugated to the staple entity at the 5′ end of the oligonucleotide and comprises a third Target nucleotide sequence and a restriction enzyme cleavage site positioned 3′ of the third Target nucleotide sequence.

In embodiments, the second oligonucleotide of the second oligonucleotide-conjugated splint entity is conjugated to the splint entity at the 3′ end of the oligonucleotide and comprises a second Target nucleotide sequence, a restriction enzyme cleavage site located on the 5′ side of the second Target nucleotide sequence, and an additional nucleic acid sequence positioned 5′ of the restriction enzyme cleavage site. In embodiments, complementary DNA sequences on the second oligonucleotide-conjugated splint entity and the third oligonucleotide-conjugated staple entity hybridize to form a double-stranded DNA restriction site.

In embodiments, the surface has two capture oligonucleotides immobilized thereon. In embodiments, the first capture oligonucleotide comprises a sequence that is complementary to the 5′-most portion of the oligonucleotide of the second oligonucleotide-conjugated splint entity and is capable of ligating at its 5′ end to the 3′ end of the oligonucleotide of the third oligonucleotide-conjugated staple entity. In embodiments, the second capture oligonucleotide comprises a sequence that is complementary at its 3′ end to the UDG1 labile linkage sequence on the first oligonucleotide-conjugated capture entity.

In embodiments of the kit, each oligonucleotide-conjugated entity is specific for a different EV surface marker. In embodiments, the first oligonucleotide of the first oligonucleotide-conjugated capture entity comprises a 5′ first Target nucleotide sequence, and a UDG1 labile linkage sequence located 3′ of the first Target nucleotide sequence.

In embodiments of the kits for isolating multimarker EVs, the kits further comprise dye-conjugated in situ probes that are complementary to the Target sequences. In embodiments, a different fluorescent label is attached to each probe directed to a different Target sequence. In other embodiments, the kits further comprise probes that are biotinylated and are complementary to the Target sequences. These biotinylated probes allow for subsequent EV pulldown or recapture onto a streptavidin surface for additional analyses including ECL assays.

In embodiments, the present disclosure provides a kit for determining surface markers of a surface marker displaying agent (SMDA), the kit comprising, in one or more vials, containers, or compartments: a capture reagent; an anchoring reagent comprising an anchoring oligonucleotide; a first binding reagent comprising a first conjugating oligonucleotide; a second binding reagent comprising a third conjugating oligonucleotide; a first detection oligonucleotide comprising: (i) a first primer site; (ii) a first barcode sequence; (iii) a first blocker complement sequence; and (iv) a first hybridization sequence, wherein the first detection oligonucleotide and the anchoring oligonucleotide comprise complementary nucleotide sequences; a second detection oligonucleotide comprising: (i) a second hybridization sequence; (ii) a third hybridization sequence comprising a second barcode sequence; and (iii) a fourth hybridization sequence; a third detection oligonucleotide comprising: (i) a fifth hybridization sequence; (ii) a second blocker complement sequence; (iii) a third barcode sequence; (iv) a second primer site; and (iv) a third primer site; and an oligonucleotide insert.

In embodiments, the first hybridization sequence and the second hybridization sequence are complementary; the fourth hybridization sequence and the fifth hybridization sequence are complementary; and the third hybridization sequence is complementary to the oligonucleotide insert.

In embodiments, the first conjugating oligonucleotide and the second detection oligonucleotide comprise complementary nucleotide sequences. In embodiments, the second conjugating oligonucleotide and the third detection oligonucleotide comprise complementary nucleotide sequences.

In embodiments, the kit further comprises a first adaptor oligonucleotide. In embodiments, the first adaptor oligonucleotide comprises (i) a first sequence complementary to at least a portion of the second detection oligonucleotide and (ii) a second region complementary to the first conjugating oligonucleotide.

In embodiments, the kit further comprises a second adaptor oligonucleotide. In embodiments the second adaptor oligonucleotide comprises (i) a first region complementary to at least a portion of the third detection oligonucleotide and (ii) a second region complementary to the second conjugating oligonucleotide.

In embodiments, the kit further comprises at least two blocker oligonucleotides, wherein at least a portion of the first hybridization sequence is complexed or capable of being complexed with a first blocker oligonucleotide and at least a portion of the fifth hybridization sequence is complexed or capable of being complexed with a second blocker oligonucleotide.

In some embodiments, the kit further comprises at least two blocker oligonucleotides, wherein at least a portion of the first hybridization sequence is complexed with a first blocker oligonucleotide and at least a portion of the fifth hybridization sequence is complexed with a second blocker oligonucleotide.

In some embodiments, the third detection oligonucleotide further comprises a third primer site. In embodiments, the kit further comprises a strand-displacement primer and strand-displacement polymerase. In embodiments, the strand-displacement polymerase comprises a Klenow fragment.

In embodiments, the kit further comprises a wash buffer. In embodiments, the wash buffer comprises Arginine. In embodiments, the kit further comprises a surface.

In embodiments of any of the above kits, the surface comprises a particle, a bead, or a surface of a culture dish, culture well, or plate. Suitable surfaces are described herein.

In embodiments of any of the above kits, the kit comprises one or more buffers. In embodiments, the kit comprises one or more of a wash buffer, an assay buffer, and a read buffer. In embodiments, the same buffer can be used for the wash, assay, and detection (i.e., “read”) steps. In embodiments, the kit comprises a Tris buffer and/or a phosphate buffer. Non-limiting examples of wash buffers, assay buffers, and/or read buffers include phosphate buffer, Tris buffer, HEPES buffer, and the like. In embodiments, the wash buffer and/or the read buffer comprises a surfactant. In some embodiments, the surfactant is TRITON-X. In embodiments, the surfactant is TWEEN-20. In embodiments, the wash buffer and/or the read buffer comprise a co-reactant. In embodiments, the co-reactant is tripropylamine (TPA). In embodiments, the read buffer is a Tris buffer comprising TRITON-X and TPA. In embodiments, the wash buffer and/or the read buffer comprises Arginine. In embodiments, the wash buffer and/or the read buffer comprise about 500 mM of Arginine.

### Automated High Throughput EV Detection

The disclosure further provides an automated version of the detection methods of the disclosure using a high-throughput robotic liquid handling system. This system allows simultaneous preparation of up to 480 samples with accuracy and reproducibility unmatched by a human operator. In embodiments, the automated system is a free-standing, fully integrated system for carrying out immunoassays using ECL technology. This system, capable of simultaneously running up to five 96-well assay plates, consists of a robotic lab automation workstation for liquid handling and plate manipulation, physically integrated with an ECL reader.

In embodiments, the workflow conducts the methods of the disclosure with minimal human intervention. In embodiments, a single 96-well source plate is loaded with plasma samples in each well. Sample is dispensed along with appropriate diluents into the desired capture plate having capture reagents attached to the surface. Plates are incubated on shakers while EVs are captured. Next, the plates are washed and the binding reagents are then added and incubated. Plates are washed again followed by addition of oligonucleotide insert and ligase. Plates are washed again followed by addition of primers and reagents for generating a single output oligonucleotide. The operator only has to set up the source plate, and load reagents into the instrument. No further intervention is required. In embodiments, the procedure is run three days in a row on the same samples with the same plate and reagent lots to assess run-to-run variability.

### Screening for Multiple Markers to a Same Target (e.g., SMDA)

One of the challenges associated with multi-marker isolation (i.e., using two or more capture and/or detection reagents) of target molecules, for example, macromolecules such as proteins, protein complexes, or EVs, is the need to efficiently identify multi-marker signatures that correspond to the targets of interest. Multiple markers may need to be identified in any immunoassay requiring three or more binding reagents on a single protein, protein complex, or large macromolecule such as EV. For example, multiple binding reagents or entities may be desired for binding to different epitopes on the same protein. Multiple binding reagents or entities may also be desired for binding to different surface markers on the same EV. In another example, multiple binding reagents or entities may be desired for binding to the same epitope in a multimeric target (e.g., protein or protein complex), and it may be desirable to use one or more of the same binding reagents or entities for binding to the same epitope in different monomers of the multimeric target. The present disclosure provides methods of screening large libraries of binding reagents or entities to identify combinations of binding reagents or entities that bind to the same target. In embodiments, the target is an EV, and the binding reagents or entities can target different surface markers (e.g., proteins) on the same EV. In embodiments, the target is a large macromolecule, for example, a single protein, and the binding reagents or entities can target different epitopes on the same protein. In embodiments, the target is a protein complex comprising one or more of the same protein monomers, and the binding reagents or entities can target the same epitope on different monomers.

A non-limiting, exemplary process for a method of determining surface markers on a target is illustrated in WO 2019/222708, and can also be used with surface marker displaying agents described herein and includes:

1. Creating a binding reagent library. This includes hybridizing binding reagents, and in some embodiments an anchoring reagent, to three different types of detection sequences, each of which comprises a unique barcode oligonucleotide sequence. In embodiments, each of the three types of detection sequences allow all combinations of binding reagents to be tested. In embodiments, the combinations of at least three binding reagents comprise three different binding reagents. In embodiments, the combinations of at least three binding reagents comprise two or more of the same binding reagent, wherein each binding reagent is coupled to each of the three types of detection sequences. In embodiments, the combination of three binding reagents comprise two or three of the same binding reagent, wherein each binding reagent, and in some embodiments the anchoring reagent, is hybridized to each of the three types of detection sequences. In embodiments, the barcode sequence is unique to each detection sequence combination, allowing the binding reagents to be identified by sequencing.

2. Contacting the binding reagents and oligonucleotide insert with the SMDA and incubating to allow binding of the binding reagents to the SMDA.

3. Capturing the SMDA bound to the binding reagents and washing away unbound binding reagents. In embodiments, the binding reagent is bound to a solid support (e.g., a bead or surface), for example, if one of the two or three binding reagents is biotinylated, and the solid support is coated with streptavidin. The SMDA can also be captured, for example, using an additional, common binding marker, such as a tetraspanin on an EV.

4. Wherein when at least one first binding reagent, one second binding reagent, and one third binding reagent is bound to the same SMDA, complementary regions of the detection sequences hybridize (see, e.g., FIG. 11). In embodiments, when at least one capture reagent, one first binding reagent, and one second binding reagent is bound to the same SMDA, complementary regions of the detection sequences hybridize (see, e.g., FIG. 12).

5. Adding polymerase and ligase to the reaction mixture to join the first, second, and third detection sequences, creating an output oligonucleotide that includes all three barcode sequences. In embodiments, the polymerase is a strand-displacement polymerase (e.g., Klenow fragment).

6. Amplifying and releasing/displacing the output oligonucleotide. In embodiments, the newly displaced output oligonucleotide is annealed with primers in the solution and is used as a template for further amplification.

7. Adding a polymerase, a forward primer, and reverse primer complementary to the first primer site and the second primer site and further amplifying the output oligonucleotide by PCR. In embodiments, the first primer site and the second primer site act as sequencing primer sites. In embodiments, the method comprises incorporating additional sequencing primer sites.

8. Sequencing the output oligonucleotides to identify combinations of the barcode oligonucleotide sequences.

In embodiments, each sequencing read contains three barcode oligonucleotide sequences, which will be mapped to the identity of the binding reagent. In embodiments, the binding reagents are hybridized to the unique detection sequences with adaptor oligonucleotides as disclosed herein. In embodiments, the adaptor oligonucleotide comprises a first sequence complementary to a conjugating oligonucleotide attached to the binding reagent, and a second sequence complementary to at least a portion of the detection sequence. In embodiments, the frequency of a specific combination of binding reagents will be related to the abundance of the three markers (e.g., surface markers on an EV or epitopes on a protein). In embodiments, the abundance of a single marker (e.g., surface markers on an EV or epitopes on a protein) is determined from the frequency with which the marker is identified from the barcode oligonucleotide sequencing results. In embodiments, multiple binding reagents targeting the same marker is compared using the barcode oligonucleotide sequencing results. For example, the highest affinity binding reagent can be identified as the binding reagent most represented by its barcode in the sequencing data.

In embodiments, the sequencing is performed with high-throughput sequencing. In embodiments, the sequencing produces at least 10⁶ reads. In embodiments, the sequencing produces at least 10⁷ reads. In embodiments, the sequencing produces at least 10⁸ reads. In embodiments, the sequencing produces at least 10⁹ reads.

In embodiments, the plurality of unique binding reagents or unique binding entities comprises at least ten unique binding reagents or unique binding entities. In embodiments, the plurality of unique binding reagents or unique binding entities comprises about 10 to about 1000 unique binding reagents or unique binding entities. In embodiments, the plurality of unique binding reagents or unique binding entities comprises about 10 to about 100 unique binding reagents or unique binding entities.

In embodiments, the multiplexed method is conducted in solution.

In embodiments, the methods described herein enable combinatorial screening and/or isolation of more than 10, more than 20, more than 30, more than 40, more than 50, more than 60, more than 70, more than 80, more than 90, more than 100, more than 500, or more than 1000 binding reagents or entities (e.g., antibodies) for EV surface markers in a single reaction. Thus, in embodiments, at least 10³, 20³, 30³, 40³, 50³, 60³, 70³, 80³, 90³, 100³, 500³, or 1000³ possible three-marker combinations can be screened in a single reaction. In embodiments, the multiple (e.g., three) binding reagents or entities also serve as a secondary tether, allowing selective removal of EVs lacking the combination of the three surface markers, thereby providing additional specificity.

In embodiments, the surface markers are identified by next-generation sequencing of the barcode oligonucleotide sequences associated with the binding reagents or entities specific for the surface markers. In embodiments, the same multiple (e.g., three) binding reagents (e.g., antibodies) identified by next-generation sequence are used for isolation of the EV, thereby simplifying reagent preparation and ensuring that the binding reagents (e.g., antibodies) behave similarly during both screening and isolation processes.

Provided herein are methods and compositions for improved identification or quantification of nucleic acids in next-generation sequencing assays and other assays using spike-in nucleic acids. In some cases, spike-in nucleic acids have special characteristics such as specific sequence, length, GC content, degree of degeneracy, degree of diversity, and/or known starting concentration. The use of such spike-in nucleic acids includes absolute abundance determination, relative abundance determination, abundance normalization, universal quantification, bias control, sample identification, cross-contamination detection, information transfer efficiency, reagent tracking, diversity loss normalization, absolute or relative loss determination, quality control and many other applications can be enabled and improved. Spike-in nucleic acids provided herein also include specially designed carrier nucleic acids that have the ability to increase the total concentration of nucleic acids in a sample, but avoid detection by sequencing or other assays. The methods provided herein are particularly useful for detecting trace amounts of nucleic acids from clinical samples such as plasma, but may also be used to detect other types of targets.

Provided herein is a “primer-limited” PCR assay or method of PCR “compression”. Multiplex PCR saturation is an undesirable phenomenon that may occur in a multiplex assay when the amplification of the more abundant gene saturates the thermostable DNA polymerase, suppressing the amplification of the less abundant gene. The remedy for saturation is a reduction of the PCR primer concentration for the more abundant target, termed “primer limitation.” The primer-limited concentration should be sufficient to enable exponential amplification, but sufficiently low that the primer is exhausted before the PCR product accumulates to a level that starves amplification of the less abundant target.

In embodiments, the EVs are isolated from monocytes. In embodiments, the EVs are isolated from B cells. In embodiments, the EVs are isolated from CD4+ T cells. In embodiments, the EVs are isolated from CD8+ T cells. In embodiments, the EVs are isolated from vascular endothelial cells.

It is to be understood that this disclosure is not limited to the particular compositions or process steps described, as such can, of course, vary. As will be apparent to those of skill in the art upon reading this disclosure, each of the individual aspects and embodiments described and illustrated herein has discrete components and features which can be readily separated from or combined with the features of any of the other several aspects without departing from the scope or spirit of the present disclosure. Any recited method can be carried out in the order of events recited or in any other order which is logically possible.

All references cited herein, including patents, patent applications, papers, textbooks and the like, and the references cited therein, to the extent that they are not already, are hereby incorporated herein by reference in their entirety for all purposes.

Sequences

First Primer Site (Forward Primer):
AACTCCATCGAACTAATGCC (SEQ ID NO: 1).
UMI:
NNNNNNNNN (SEQ NO: 2); N = any DNA base.
First Barcode:
TAGCAAGAGAGTCTTA (SEQ ID NO: 3).
Second Barcode:
CATTCACCGTGGAAGCAGTC (SEQ ID NO: 4).
Second Barcode Reverse Complement:
GTAAGTGGCACCTTCGTCAG (SEQ ID NO: 5).
Third Barcode:
TAATCCCACGCTGTGA (SEQ ID NO: 6).
Second Primer Site (Reverse Primer):
ATTCCCTCGTAACGGAGAAG (SEQ ID NO: 7).
Second detection oligonucleotide (Splint oligo):
CAGAATCATTCACCGTGGAAGCAGTC ATTAGG (SEQ ID NO: 8).
Anchoring Oligonucleotide (PLLSD Capture Anchor):
CCGCCCTTCGCCTACATTATGGACTTCGACATAATTGACT (SEQ ID
NO: 9).
First detection oligonucleotide (5-prime oligo):
ATAATGTAGGCGAAGGGCGGNNNNNNNNNNGCCTTGTCTTA (SEQ ID
NO: 10);; N = any DNA base.
Second detection oligonucleotide (Splint oligo):
GGATTACTGACAGAAGAGAAGCTTACTAAGACTGGTCCTACGAGACATC
GGT (SEQ ID NO: 11).
Oligonucleotide insert:
GTAAGCTTCTCTTCTGTCAG (SEQ ID NO: 12).
Third detection oligonucleotide (3-prime oligo):
TAATCCGCATGNNNNNNNNNNTGGTCCTACGAGACATCGGTATCAGGTT
CACAGCAG (SEQ ID NO: 13); N = any DNA base.
Strand Displacement Primer:
CTGCTGTGAACCTGAT (SEQ ID NO: 14).
Second or Third Conjugating Oligonucleotide
(PLLSD Detector Anchor):
ACCGATGTCTCGTAGGACCA (SEQ ID NO: 15).
First Blocker Oligonucleotide (5-prime blocker):
TAAGACAAGGC (SEQ ID NO: 16).
Second Blocker Oligonucleotide (3-prime blocker):
CATGCGGATTA (SEQ ID NO: 17).

### EXAMPLES

Reference is made to specific examples illustrating the constructs and methods above. It is to be understood that the examples are provided to illustrate rather than limit the scope of various embodiments of the disclosure.

### Example 1—Optimized Proximity Ligation-Ligation (PLL) Methods for Multimarker EV Analysis in Normal and Cancer Samples

The PLL method and oligonucleotide configuration shown in FIG. 1A, FIG. 1B and FIG. 1C was used to generate the data in the present Example. The 5′ conjugate comprised a first antibody conjugated to an oligonucleotide with sequences for a forward primer, a first unique molecular identifier sequence, a first barcode and a first common overlap region (hybridization region) hybridized to a first blocker oligonucleotide. In this example, the 5′ end of the 5′ conjugate oligonucleotide was attached to the first antibody. The splint conjugate comprised a second antibody conjugated to an oligonucleotide with sequences for a first common overlap region, a second barcode and a second common overlap region. In this example, the 3′ end of the splint conjugate oligonucleotide was attached to the second antibody. The 3′ conjugate comprised an antibody conjugated to an oligonucleotide with sequences for a reverse primer, a second unique molecular identifier sequence, a third barcode and a second common overlap region (hybridization region) hybridized to a second blocker oligonucleotide. In this example, the 3′ end of the 3′ conjugate oligonucleotide was attached to the third antibody.

As shown in FIG. 1A, a capture antibody immobilized to a bead surface was used to capture the EV. The 5′ conjugate, splint conjugate and 3′ conjugate were added to the EV captured on the bead, and the antibodies of each conjugate bound to their corresponding surface marker antigens on the EV. The labels 5′, 3′ and 3′ indicate the end of the nucleic acid conjugated to the antibody. The blocker oligonucleotides hybridized with the 3′ and 5′ conjugates comprised a 3′ overhang to avoid the formation of double-stranded blunt ends. As shown in FIGS. 1A and 1B, after the first blocker oligonucleotide was displaced, the first common overlap region of the 5′ conjugate hybridized with the first common overlap region on the splint conjugate. Similarly, after the second blocker oligonucleotide was displaced, the second common overlap region on the splint conjugate hybridized with the second common overlap region on the 3′ conjugate. As shown in FIGS. 1A and 1B, an insert oligonucleotide that hybridizes to the unhybridized region of the splint construct was added and ligase was used to seal the construct into a single output oligonucleotide. Primers binding to the forward primer and reverse primer sites were added to amplify the single output oligonucleotide by PCR which was then sequenced as shown in FIGS. 1A and 1C.

Sequence data was analyzed by comparing each barcode to a lookup table to identify the three surface marker binding agents that were conjugated to the three oligonucleotides composing each full-length amplicon. Thus, the sequencing data can be reduced to a list of three marker combinations or permutations. Sequencing reads with identical UMIs are binned and treated as a single read. The number of sequencing reads with identical combinations was determined for each unique combination or permutation to assess the frequency of each combination or permutation in the library. This frequency distribution was used to assess the frequency of the corresponding three marker combinations on the SMDAs in the original sample. Three-marker combinations that are determined to be enriched on EVs or other SMDAs from a CRC subject but not on EVs or SDMAs of a normal subject were selected as specific indicators of that EV population. These three marker combinations may be used in assays to specifically detect or quantify that population or may be used to specifically enrich or purify that population from a mixed population of SDMAs or from a sample with other non-SDMA contaminants.

### Materials and Reagents

TABLE 2

| Item | Lot Number | Catalog Number | Notes |
|---|---|---|---|
| H2O | 725951 | | |
| PBS | 725645 | | |
| Diluent 52 | 10MAY2024 EAK | | |
| Pierce streptavidin beads | ZD387021 | 88816 | |
| Diluent 100 | Y0270333 | R50AA-2 | |
| PBST | 19AUG2024 MA & 29AUG2024 LH | | |
| LIGASE | Y0570008 | R82AC-1 | |
| IgG BLOCKER | 202209150005 | | |
| Proteinase K | 10213809 | | |
| 18% BSA | 12JAN2024 MA | | for Ab diluent |
| 18% BSA | 12JAN2024 MA | | for Assay diluent |
| HAMA BLOCKER | 11JUL2022 LH | | |
| SALMON DNA | 0000269978 | SLCR3535 | |
| REACTION BUFFER A | 486671 | | |
| MONOBASIC | 12JAN2024 LH | | |
| DIBASIC | 04AUG2021 EG | | |
| WASH BUFFER 20X | Y0510039 | | |
| Low Salt WASH | 19AUG2024 MA | | |
| Arginine Wash | 19AUG2024 LH | | |
| 1X MSD WASH BUFFER | 19AUG2024 MA | | |
| TWEEN 20 | ZF387174 | THERMO 85113 | |
| SODIUM PHOSPHATE | P07J504 | THERMO J62152.K2 | |
| BUFFER 0.2M | | | |
| SYBR GREEN | 2930508 | THERMO 100029284 | |

TABLE 3

| Cell Line EVs | Lot | Notes |
|---|---|---|
| Huvec | RN 07 May 2021 | 15X Jumbosep |
| Jurkat | 038H00101.00121SUP01.170901 | 1X |
| DiFi | 11AUG2023 HB | 10X TFF |
| HLM-2 | 028H00101.06710SUP01.170712 | 1X |
| HCT15 | RN 01 SEP 2020 | 10X TFF |
| U87MG-Diff | RN 21 APR 2020 | 1X, 10 days of |
| | | differentiation |
| THP1_diiff | A00R1153 31 JUL 2025 | 1X |
| RPMI-8226 | 065H00101.00101SUP01.180928 | 1X |
| MOLT-4 | 052H00101-00101SUP01-151218 | 1X |

TABLE 4

| | | | Catalog |
|---|---|---|---|
| Antibody | Lot Number | Clone | Number |
| Biotin-CD41 | 545469 &540424 | MEM-06 | |
| Biotin-CD61 | 19jul2024 MA | VLP2 | |
| Biotin-CD9 | B389553 | HI9a | 312112 |
| Biotin-CD63 | B377351 | H5C6 | 353018 |
| Biotin-CD81 | B368067 | 5A6 | 349514 |

### Procedure Overview

The plasma samples derived from subjects were purified by Captocore purification, as described in the art. Platelet EV depletion of the purified plasma samples was performed using anti-CD41-beads and CD61-beads pooled together. EVs were further isolated by a pull-down method using TSPAN-coated beads. Antibody and blocker aggregates were removed by centrifugation for 30 min at 21,000 g.

The PLL assay was performed in a reduced reaction volume from 100 μL to 25 μL and 11 nucleotide oligo blockers specific to each of the 3′ and 5′ conjugates were added. No bead release was performed during King Fisher programs, but an 500 mM Arginine wash step was added after detection. Each enzymatic reaction concentration and duration in the PLL method was optimized.

For library preparation PCR compression was used to normalize the samples and only cell line control samples were diluted prior to the PCR. Next Generation Sequencing was performed in the P2 flow cell at 100 cycles.

### Bead Coating Protocol

TABLE 5

| | |
|---|---|
| 1. | Vortex magnetic streptavidin-coated beads very well |
| 2. | Take a 2 mL tube and fill with 1 mL phosphate buffered saline with Tween 20 (PBST) |
| 3. | Add appropriate total amount of beads needed to the 1 mL of PBST |
| 4. | Add to magnetic rack for 5 minutes |
| 5. | Pipette out as much PBST as possible without disturbing the beads |
| 6. | Repeat wash once more with PBST |
| 7. | After second wash with PBST, add 1 mL of Diluent 100 to tube while it's still in the rack |
| 8. | Remove tube from rack and vortex well |
| 9. | Put tubes with Diluent 100 + beads back on magnetic rack |
| 10. | Prepare antibodies by adding appropriate amount into Diluent 100 based on calculations |
| 11. | Take out Diluent 100 wash from bead tube on magnetic rack |
| 12. | Add 1 mL of antibody-diluent solution needed to the beads |
| 13. | Vortex tube well and add back into the rest of the antibody-diluent solution |
| 14. | Put on rotisserie for 2 hours at room temp |
| 15. | Move rotisserie to 4 °C walk-in and let tubes mix overnight |

### PLL Experiment Protocol

TABLE 6

| | |
|---|---|
| 1. | Take out pre-prepared beads from 4 °C and put on rotisserie. |
| 2. | Make assay diluent with human anti-mouse antibody (HAMA) blockers |
| 3. | Prepare sample plate by adding 50 μL of assay diluent and 50 μL of each sample |
| 4. | Prepare 2 deep well wash plates with 1000 μL in each well of PBST for each PLL plate. |
| 5. | Add 100 μL per well of bead solution to each bead plate. |
| 6. | Wash beads in plates on King Fisher instrument. |
| 7. | Put bead-sample plate on a shaker for 2 hours at 705 rpm at room temperature (RT). |
| 8. | Antibody diluent is 6.8X concentrated in blockers and added to each individual pool of detector |
| | previously diluted in 150 mM NaCl resuspension buffer. |
| 9. | Prepare detector solution by mixing appropriate 5′ mix, insert mix, and 3′ mix. |
| 10. | Prepare 2 shallow plates with 200 μL of PBST in each well. |
| 11. | Add 20 μL per well of detect solution in a skirted PCR plate. |
| 12. | Wash beads in plates on King Fisher instrument. |
| 13. | Put released detector plates on an Eppendorf shaker for 1 hour at 1800 rpm at RT. |
| 14. | Prepare ligation solution with T4 DNA ligase. |
| 15. | Prepare 3 shallow wash plates with 200 μL per well. |
| 16. | Load 20 μL per well of the ligation solution. |
| 17. | Wash beads in platers on King Fisher instrument. |
| 18. | Put released ligation plates on an Eppendorf shaker for 1 hour at 1800 rpm at RT. |
| 19. | Turn on thermal shaker and set to 40 °C. |
| 20. | Prepare 1 shallow plate with 200 μL per well of PBST. |
| 21. | Prepare Proteinase K solution and load 20 μL per well of the proteinase K solution into a new |
| | plate. |
| 22. | Wash beads in plates on King Fisher instrument. |
| 23. | Place Proteinase K plate on thermal shaker set to 40 °C for 20 min at 1800 rpm. |
| 24. | Use King Fisher to release beads into a waste plate filled with PBST without any additional |
| | washes |
| 25. | The PCR plate now contains the final product without beads. |
| 26. | Place PCR plate in thermal cycler and heat to 95 °C for 15 min to inactivate proteases. |
| 27. | Store inactivated PCR plate at −20 °C. |

### PCR and Library Preparation

DNA analysis of the DNA library on the Agilent Tapestation revealed a DNA size of 261 bp for Plate A, 258 bp for Plate B, 264 bp for Plate C, and 260 bp for Plate D. Quantification of the library as demonstrated in Table 7, revealed a lower concentration than expected, suggesting lower amplification efficiency and thus less efficient PCR compression.

TABLE 7

| | | | dsDNA | | Library |
|---|---|---|---|---|---|
| Library | | | Length | MW | Conc. |
| Name/PhiX | Qubit Conc. (ng/μL) | | (bp) | (g/mol) | (nM) |
| PLL Cancer Plate A | 0.086 | 0.088 | 250 | 151850 | 0.57 |
| PLL Cancer Plate B | 0.125 | 0.127 | 250 | 151850 | 0.83 |
| PLL Cancer Plate C | 0.113 | 0.123 | 250 | 151850 | 0.78 |
| PLL Cancer Plate D | 0.120 | 0.119 | 250 | 151850 | 0.79 |

### Analysis of Cancer Samples Vs Normal Samples

Sample demultiplexing of library sequences was performed using unique dual indexing (UDI). Normalization was carried out using a Spiked full-length construct (corrects for error in the library prep and corrects for differences post-assay dilutions/amplifications between samples). The barcode sequences were identified by matching the 3 barcodes per read to a look-up table.

Phenotype Binning of each read was conducted. All phenotype binning was calculated using Spike-normalized reads. Reads can count in multiple bins, e.g. CD73 means at least one CD73 barcode is present in any of 3 positions; CD73_CD13 means at least one of each surface marker barcode is present in any position; CD73_CD73_CD13 means exactly 2 CD73 and CD13 barcodes are present in any position; CD73_CD73_CD73 means three CD73 barcodes are present in any position.

CRC samples from a population of subjects with stage IV CRC were compared to healthy control samples for all phenotype bins with the Wilcoxon test. A set of plasma samples from a first population of subjects with refractory CRC was compared to healthy control samples for all phenotype bins with the Wilcoxon test. A set of plasma samples from a second population of subjects with refractory CRC was compared to healthy control samples for all phenotype bins with the Wilcoxon test. A set of samples from Breast Cancer patients was compared to healthy control samples for all phenotype bins with the Wilcoxon test. A set of samples from Gastric and Esophageal Cancer patients was compared to healthy control samples for all phenotype bins with the Wilcoxon test.

The 100 most significantly increased bins in the first population of subjects with refractory CRC samples were selected and univariate cox regressions were computed for Overall Survival and PFS. The 100 most significantly increased bins in the second population of subjects with refractory CRC samples were selected and univariate cox regressions for Overall Survival and PFS were computed.

Each phenotype bin was treated as an independent observation and evaluated independently when comparing sample groups. 0.1 normalized counts were added to every observation before comparisons to eliminate errors arising from zeros. The median and geometric mean of each group were calculated and ratios of these were compared between samples to assess fold change.

The Mann-Whitney U-Test, also called Wilcoxon rank sum test, was selected to compare the groups since distributions were not normal. A single-tailed test was performed with the alternative hypothesis that counts were higher in cancer samples than normal samples.

Comparison of CRC Samples from First Population of Subjects with Refractory CRC and Healthy Control Samples

The bins most significantly elevated in CRC patients were highly enriched for some markers already expected, e.g. CD73, CD66e. Other markers were observed that make sense in the context of metastatic disease but had not previously observed: CD324, CD325, CD326, CD13, CD66a, CD10. Each of these was also found in combinations with CD73.

Several other markers in combinations with CD73 were observed that were unexpected. Each of these markers can be expressed on multiple cell types but the combination with CD73 gave strong evidence that these populations are secreted by endothelial cells: CD31, CD36, CD141, CD14, CD54. These markers were also found in combinations with each other, supporting the conclusion that they came from same cell type

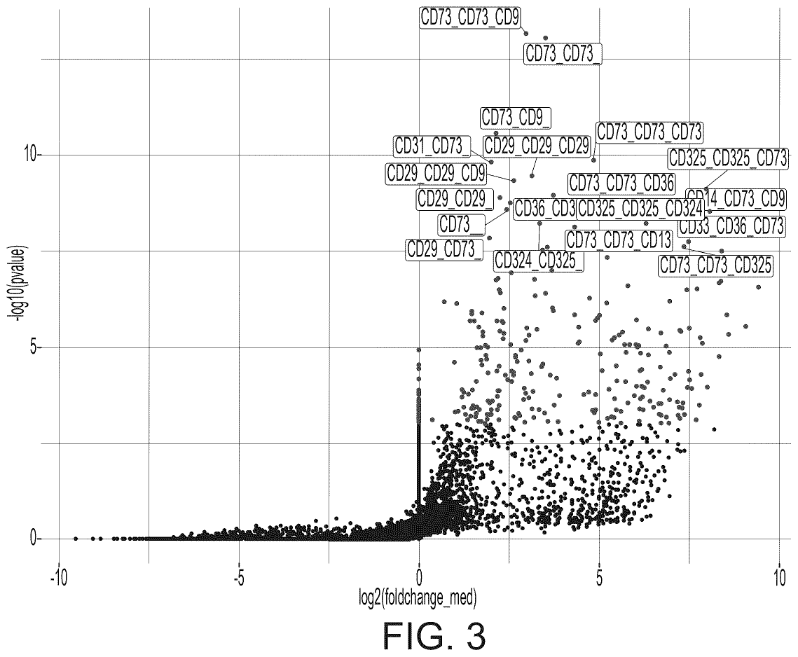

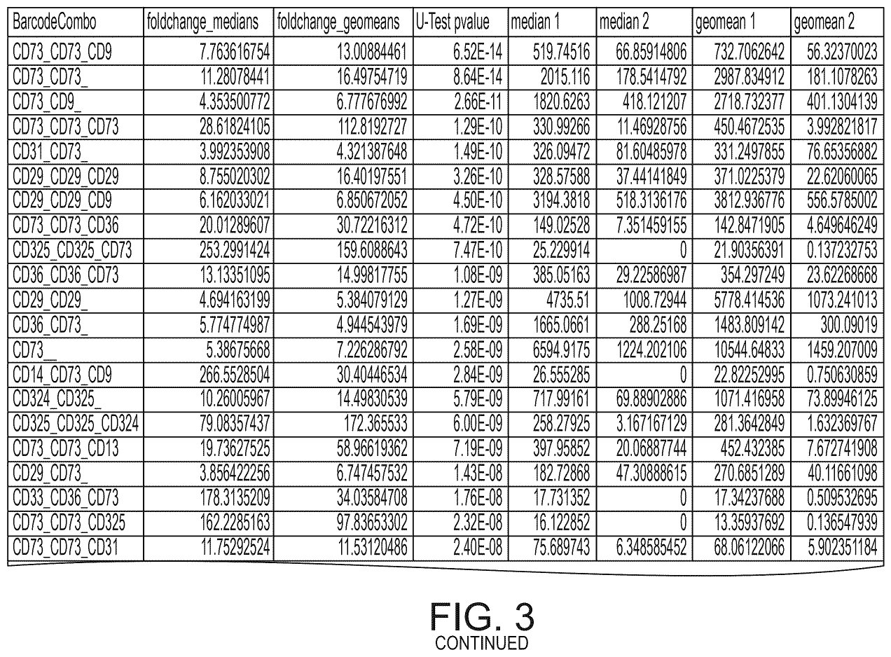

The volcano plot in FIG. 3 shows significant enrichment of many phenotype bins in CRC samples from the first population of subjects with refractory CRC versus healthy control samples. This includes combinations of epithelial markers, indicating EVs likely derived from the tumor cells and combinations of endothelial markers, indicating EVs likely derived from the tumor stroma.

Comparison of CRC Samples from Second Population of Subjects with Refractory CRC and Healthy Control Samples

The same analysis for the CRC samples from a second population of subjects with refractory CRC was performed. Most of the same markers and combinations were enriched in the CRC samples with some notable exceptions: CD26, which was found in combination with both endothelial (e.g. CD36, CD54 and epithelial markers e.g. CD66e).

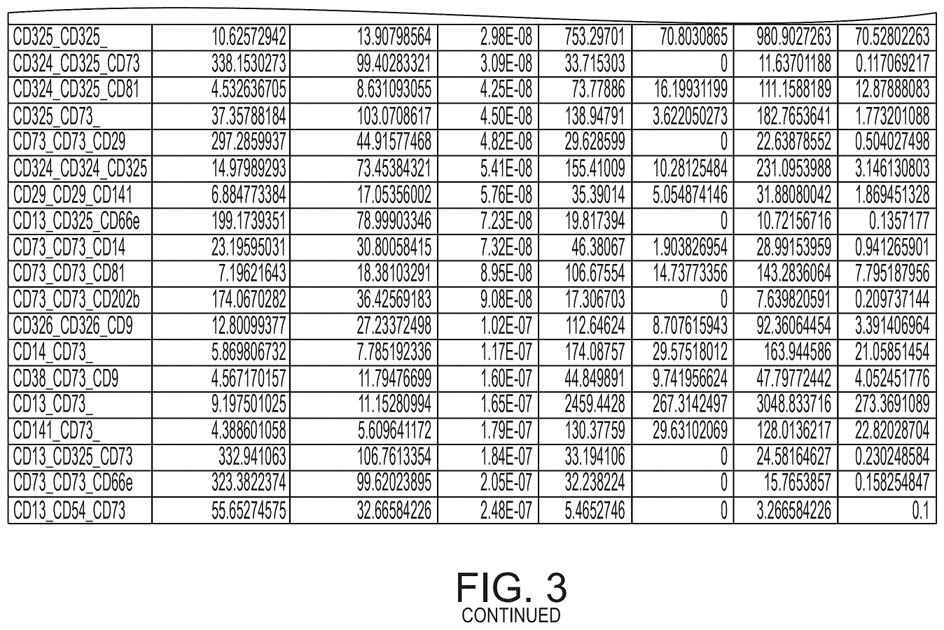

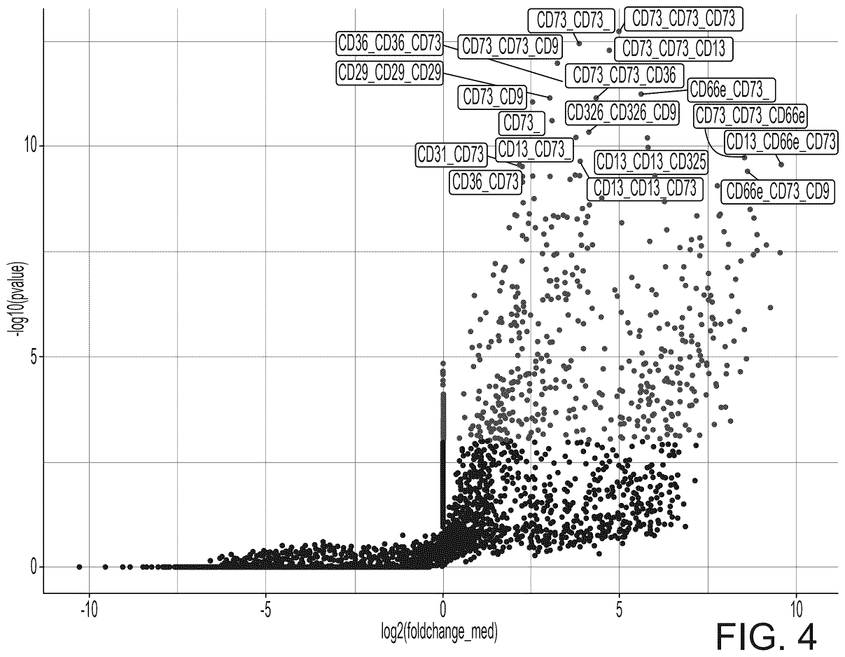

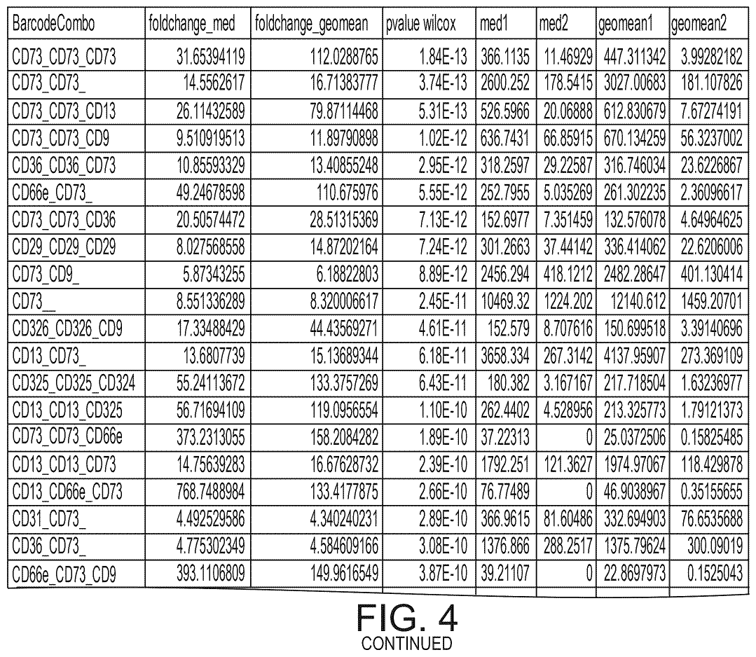

The volcano plot in FIG. 4. shows significant enrichment of many phenotype bins in the CRC samples from the second population of subjects with refractory CRC versus healthy control samples. This includes combinations of epithelial markers, indicating EVs likely derived from the tumor cells and combinations of endothelial markers, indicating EVs likely derived from the tumor stroma.

CRC Samples from Population of Subjects with Stage IV CRC Vs Healthy Control Samples

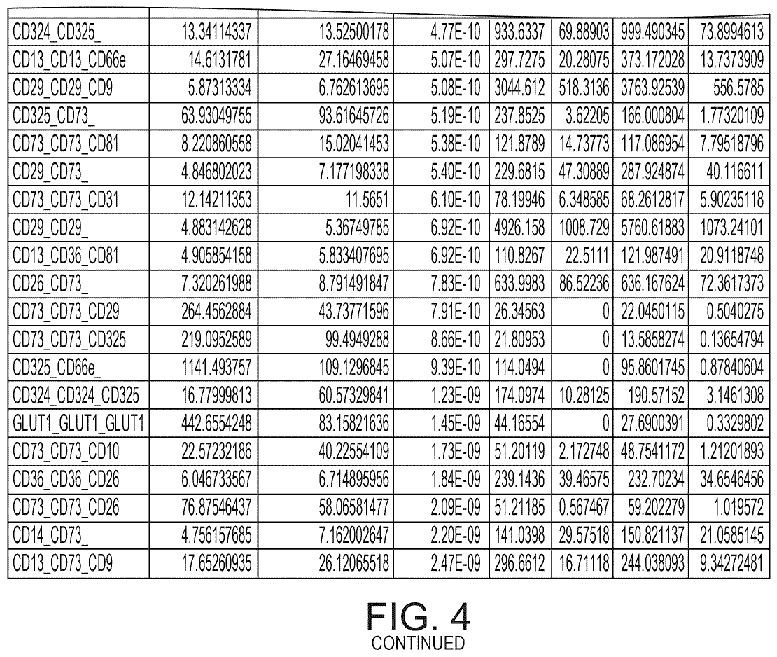

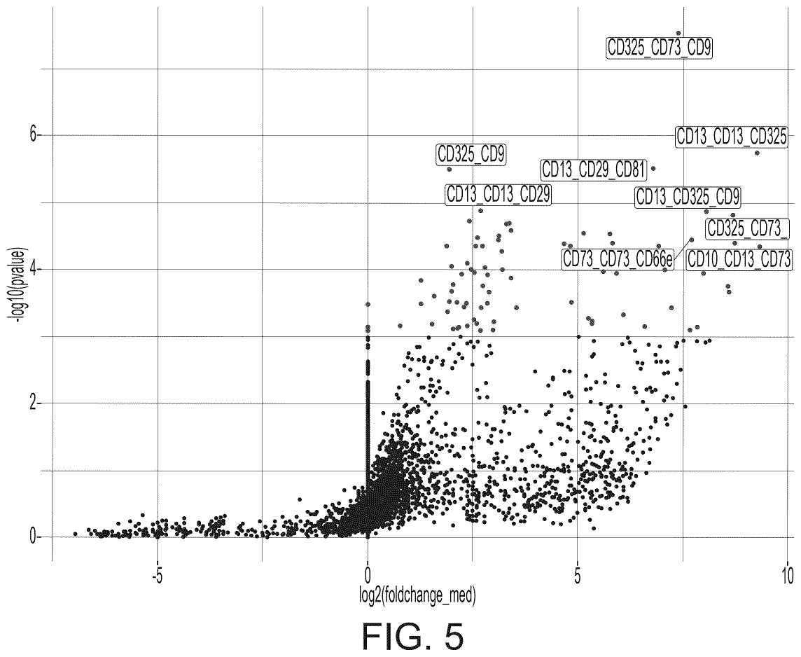

The same markers and combinations noted above were observed to be enriched in the CRC samples from the population of subjects with stage IV CRC as shown in FIG. 5.

Survival Analysis in First Population of Subjects with Refractory CRC

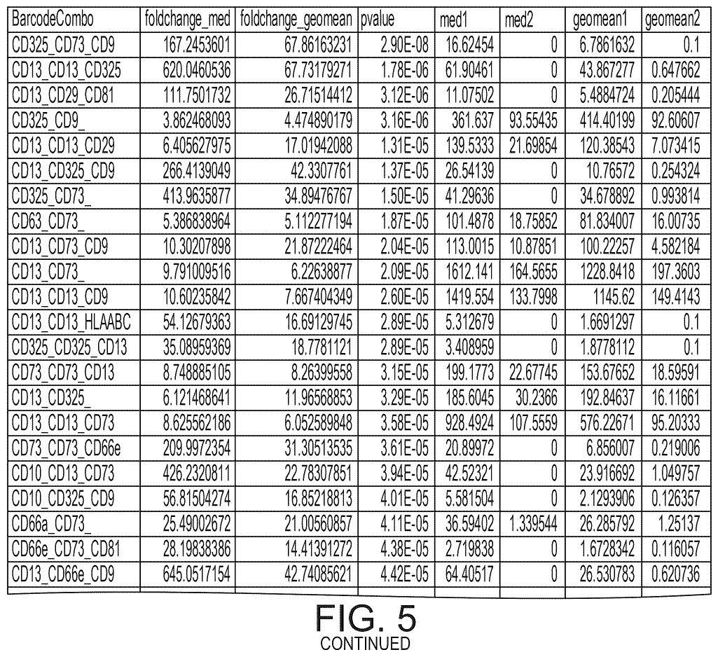

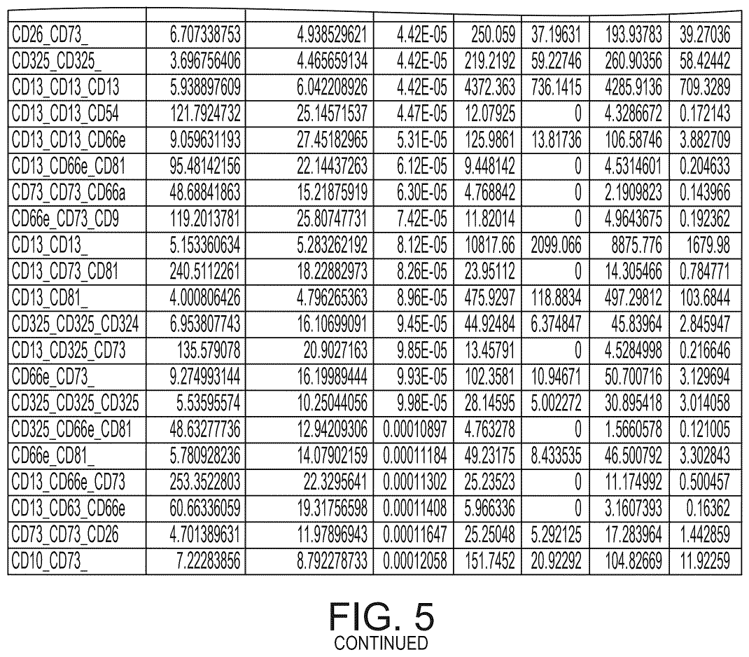

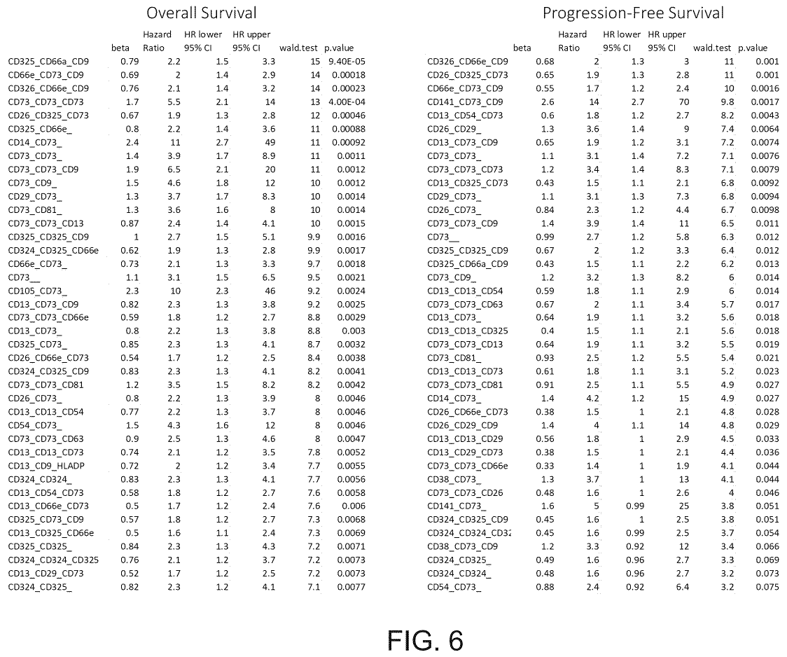

Survival analysis using Cox regression was performed for overall survival (OS) and progression free survival (PFS) in the CRC samples from the first population of subjects with refractory CRC vs. healthy control samples. The 100 phenotype bins that were most significantly increased in the CRC cases vs controls were analyzed using a univariate Cox model. The Tables in FIG. 6 show these bins sorted by p-values. Interestingly the most significant bin for OS has neither CD73 nor CD66e. However, CD66e_CD73_CD9 is highly significant for both OS and PFS. Many other combinations are prognostic.

Survival Analysis in Second Population of Subjects with Refractory CRC

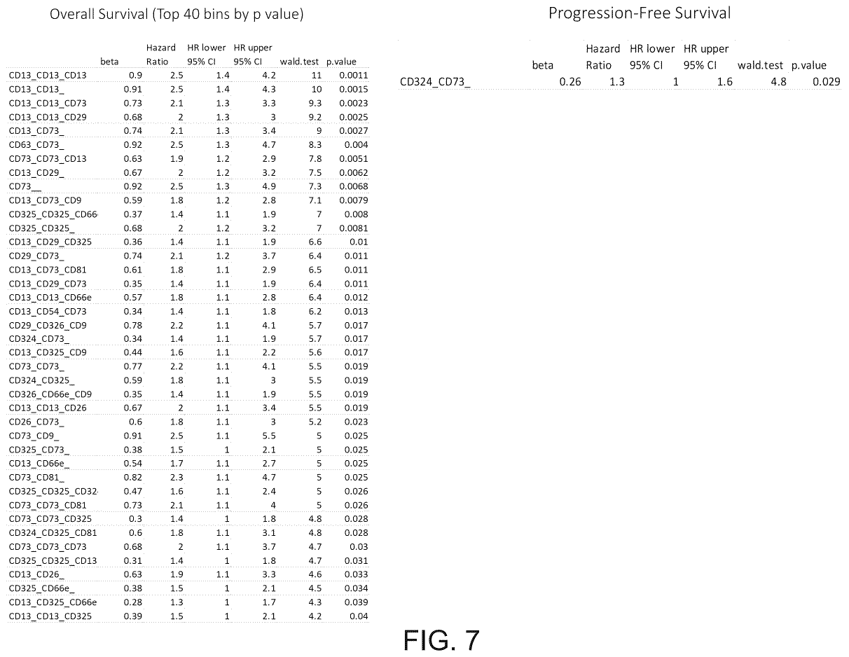

Survival analysis using Cox regression was performed for overall survival (OS) and progression free survival (PFS) in the CRC samples from the second population of subjects with refractory CRC vs. healthy control samples. The 100 phenotype bins that were most significantly increased in the CRC cases vs controls were analyzed using a univariate Cox model. The Tables in FIG. 7 show these bins sorted by p-values. While there were many significant combinations for OS, only one was significant for PFS.

### Other Cancers

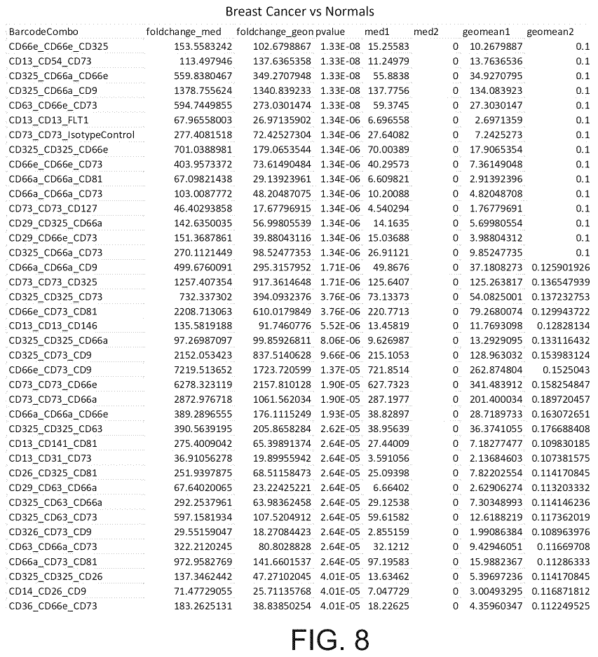

The second population of subjects with refractory CRC contained a small number of samples from patients with cancers other than colorectal, including Breast cancer and Esophageal and Gastric cancers. Mann-Whitney tests on the Breast Cancers versus the controls and the Gastric and Esophageal Cancers versus controls was performed. Many of the same combinations observed in the CRC samples were also observed here, though the fold changes were higher in the breast cancer samples than the CRC samples (FIG. 8). Markers were also observed that weren't observed as highly significant in the CRC samples, e.g. FLT1. Many of the same bins are highly elevated in gastric/esophageal cancers as well, with very high fold changes (FIG. 9). A few bins include HLADP (MHC Class II) in combination with epithelial surface markers.

MHC Class II is typically restricted to antigen presenting cells, however, there is support in the art for tumor cells expressing MHC Class II so these may be real multimarker phenotypes on tumor-derived EVs (Axelrod et al., “Biological Consequences of MHC-II Expression by Tumor Cells in Cancer,” Clinical cancer research: an official journal of the American Association for Cancer Research 25(8):2392-2402 (2019)).

### Correlation of PLL and Intact EV ECL Assays

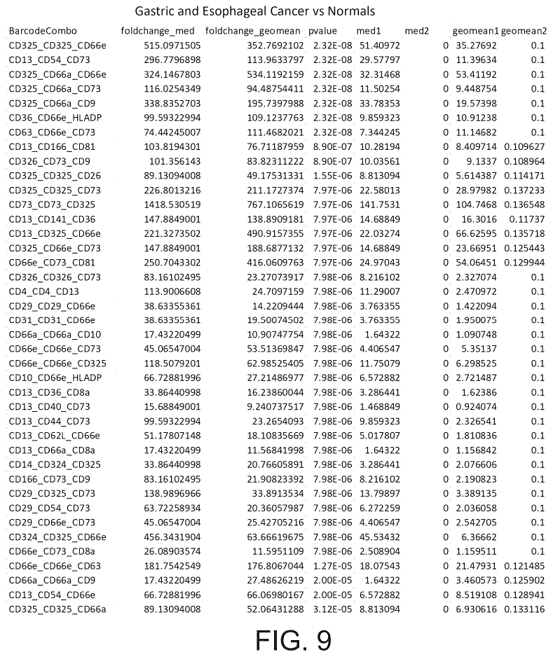

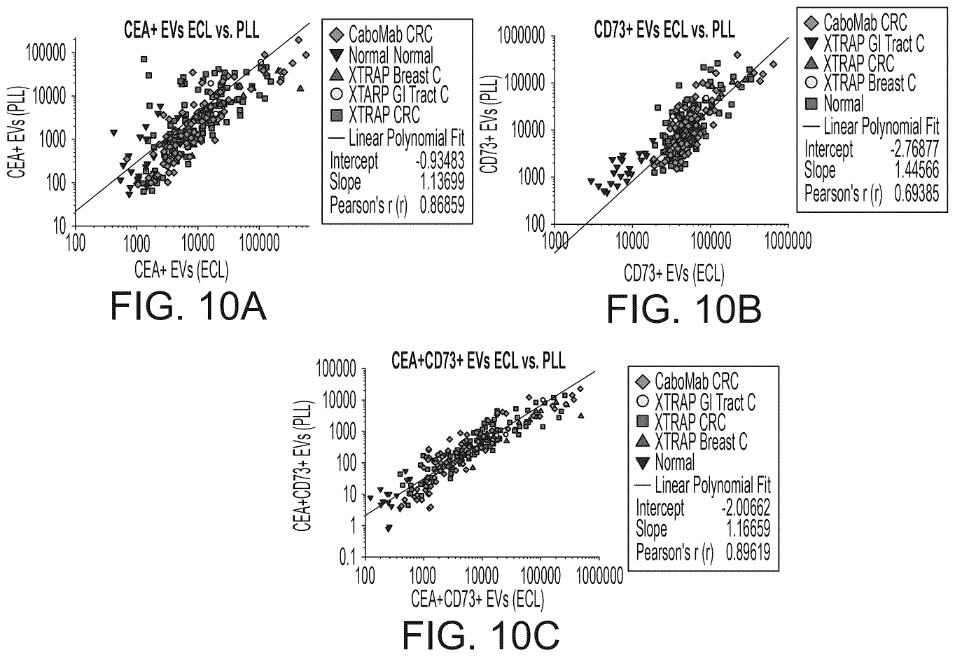

To demonstrate the quantitative performance of the PLL assay, a comparison of the signal for several bins in the PLL assays to the ECL assays previously run on these samples was conducted and good correlation was found between the two assays. The correlation results between PLL and ECL using a regular intact EV sandwich assay for detecting CEA+ EVs and CD73+ EVs (with and without TSPAN) are shown in FIG. 10A and FIG. 10B, respectively. The correlation results between PLL and ECL using an ultrasensitive intact EV assay for detecting CEA+CD73+ EVs is shown in FIG. 10C.

### Example 2: Optimized PLL Method for Multi-Marker EV Analysis in Normal and Breast Cancer Samples

The PLL method and oligonucleotide configuration shown in FIG. 1A, FIG. 1B and FIG. 1C was used to generate the data in the present Example.

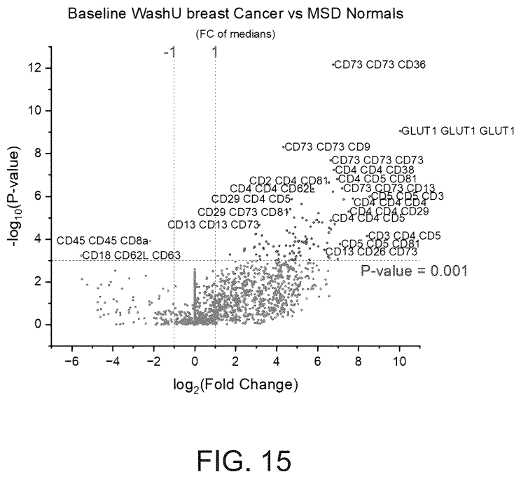

Additional 49 breast cancer plasma samples from a population of subjects with stage II, stage III and stage IV breast cancer (BC) were compared to healthy control samples. The comparison was done using log-2 transformed phenotype bins with the statistical t-test. The bins most significantly elevated in BC patients were highly enriched at the three tested time-points: 1) “Baseline”, which is the treatment naïve time point; 2) “C2D1”, which stands for “Cycle 2 Day 1 of neoadjuvant treatment”, a time-point after one complete round of neoadjuvant treatment; and 3) “Pre-Surgery”, the time-point preceding the surgery and following a complete neoadjuvant therapy. The volcano plot in FIG. 15 shows significant enrichment of many phenotype bins in the BC samples versus healthy control samples.

The bins most significantly elevated in BC patients were highly enriched for the same markers that were found elevated in CRC patients, comprising markers expected in the context of metastatic and non-metastatic cancers: CD324, CD325, CD326, CD12, CD66a, CD10, CD73, CD66e.

The bins most significantly elevated in BC patients were highly enriched in GLUT1 cancer-associated marker, in most cases in GLUT 1 was in combination with itself (GLUT1-GLUT1-GLUT1), which could be related to its self-oligomerization at the surface of extracellular vesicles.

The bins most significantly elevated in BC patients were highly enriched in markers expected in the context of the presence of immune cells, particularly T-cells, potentially infiltrating the tumor immune micro-environment, but had not been previously observed: CD4, CD5, CD3, CD2, CD8A.

### Example 3: Optimization of PLLSD

The PLL with Strand Displacement (PLLSD) illustrated in FIG. 11, FIG. 12, and FIG. 14 was developed and optimized as described in the following example. In this embodiment, clonal beads are used for capture. In this embodiment, the capture reagent on the clonal beads can be a common EV surface marker, a platelet-associated marker, a cancer-associated marker, a tumor-stromal marker, an immune marker or any relevant surface marker.

The specificity of the signal generated by PLLSD using the construct shown in FIG. 11 is illustrated in Table 8. The specificity of the signal generated by PLLSD using oligo as described in FIG. 11 was tested by replacing the capture surface marker CD81, or the splint surface marker (first binding reagent) CD81, or the 3-prime surface marker CD81 (second binding reagent) by an isotope control (IgG). The specificity was also tested by removing capture surface marker, or the splint surface marker, or the 3-prime surface marker from the mix of PLLSD reagents (“none”). The signal decreased at least 350-fold when surface marker CD81 was replaced by an isotope control. The signal decreased at least 1660-fold when surface marker CD81 was removed from the reagents.

TABLE 8

| | | | | qPCR data (triplicates) | | | |
|---|---|---|---|---|---|---|---|
| | Capture | Splint | 3Prime | Expi293 | | PBS | |
| Enzyme | Ab | Ab | Ab | Avg Ct | % CV | Avg Ct | % CV |
| Ligase | CD81 | CD81 | CD81 | 13.2 | 10% | 32.0 | 36% |
| | None | CD81 | CD81 | 28.5 | 8% | 32.3 | 16% |
| | IgG | CD81 | CD81 | 29.8 | 7% | 33.5 | 27% |
| | CD81 | None | CD81 | 23.9 | 7% | 34.6 | 36% |
| | CD81 | IgG | CD81 | 21.7 | 4% | 31.3 | 22% |
| | CD81 | CD81 | None | 29.2 | 12% | 36.1 | 48% |
| | CD81 | CD81 | IgG | 23.4 | 7% | 31.4 | 61% |
| | IgG | IgG | IgG | 33.4 | 52% | 32.8 | 9% |
| No | CD81 | CD81 | CD81 | 24.6 | 10% | 34.3 | 43% |
| Ligase | None | CD81 | CD81 | 34.6 | 38% | 34.1 | 15% |
| | IgG | CD81 | CD81 | 34.5 | 42% | 35.0 | 21% |
| | CD81 | None | CD81 | 24.7 | 6% | 34.8 | 12% |
| | CD81 | IgG | CD81 | 24.6 | 12% | 35.2 | 46% |
| | CD81 | CD81 | None | 36.1 | 33% | 36.9 | 88% |
| | CD81 | CD81 | IgG | 35.0 | 43% | 35.4 | 30% |
| | IgG | IgG | IgG | 34.8 | 32% | 35.0 | 24% |

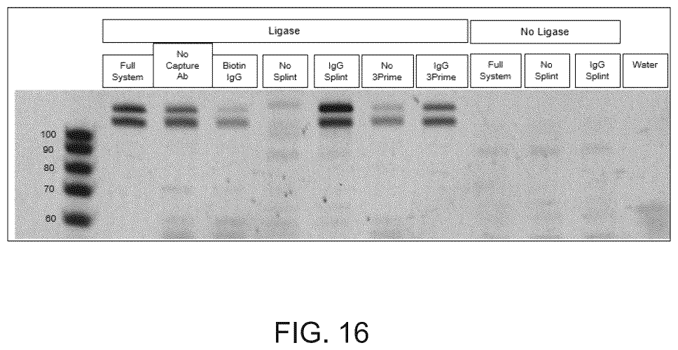

The presence of full-length signal when surface marker CD81 was removed from the reagents (FIG. 16) was attributed to a contamination of the splint detector CD81 by some 3-prime detector CD81.

The PLLSD assay was optimized through a series of modifications aimed at substantially shortening the oligonucleotides to maximize product formation, reduce non-specific signal, and lower overall costs. These modifications are illustrated in FIG. 17 and Tables 9, 10, and 11. The first detection oligonucleotide (“5′ Prime” in FIG. 17) length was shortened from 64 nucleotides to 41 nucleotides, by removing the forward primer region of 20 nucleotides, listed as “Primer 1” in FIG. 11 and FIG. 12; and by removing 3 nucleotides to the Blocker Stabilizer Region which is listed as “HYB/Blocker Site 1” in FIG. 11 and FIG. 12. The third detection oligonucleotide (“3-prime” in FIG. 17) was reduced from 87 nucleotides to 60 nucleotides, by removing the reverse primer region of 20 nucleotides, listed as “Primer 2” in FIG. 11 and FIG. 12; and by removing 3 nucleotides to the Blocker Stabilizer Region which is listed as “HYB/Blocker Site 2” in FIG. 11 and FIG. 12, and by removing 4 nucleotides from the stand displacement primer region. The effects of the aforementioned modifications were evaluated using combinations that included the CD81 marker—to represent an abundant target—and the CD44 marker—to represent a low-abundance target—on unmodified Expi293 EV samples.

Table 9 shows qPCR Ct values for full-length 3-marker combinations, demonstrating no difference in specific product expression in Expi293 EV samples between the short and long versions of both the 5′ and 3′ oligos, and no change in background signal in PBS controls.

TABLE 9

| | | | 5Prime | | | | | | | |
|---|---|---|---|---|---|---|---|---|---|---|
| | | | CD81 | | | | CD44 | | | |
| | | | Short (41 nt) | | Long (64 nt) | | Short (41 nt) | | Long (64 nt) | |
| Splint | 3Prime | | Expi293 | PBS | Expi293 | PBS | Expi293 | PBS | Expi293 | PBS |
| CD81 | CD81 | Short (60 nt) | 13.4 | 30.7 | 14.3 | 31.7 | 18.1 | 31.3 | 19.2 | 31.8 |
| | | Long (87 nt) | 13.3 | 30.6 | 14.1 | 31.0 | 18.1 | 30.7 | 18.9 | 31.0 |
| | CD44 | Short (60 nt) | 21.3 | 33.9 | 22.2 | 33.5 | 27.9 | 34.2 | 28.8 | 35.2 |
| | | Long (87 nt) | 21.1 | 33.7 | 22.1 | 34.4 | 28.8 | 33.7 | 29.5 | 35.1 |
| CD44 | CD81 | Short (60 nt) | 19.8 | 31.5 | 20.8 | 32.0 | 26.1 | 31.8 | 27.2 | 31.5 |
| | | Long (87 nt) | 19.6 | 31.0 | 20.7 | 30.9 | 26.3 | 32.0 | 27.1 | 31.4 |
| | CD44 | Short (60 nt) | 27.7 | 34.9 | 29.0 | 34.3 | 33.7 | 35.8 | 34.4 | 35.4 |
| | | Long (87 nt) | 27.2 | 34.0 | 28.5 | 33.8 | 34.4 | 35.0 | 34.9 | 33.8 |

The 3-prime oligo (third detection oligonucleotide) was shortened further by removing the 3 nucleotides between the third primer site and the hybridization region with the detector anchor (first and second conjugating oligonucleotides). PLLSD was tested with the CD81 marker by comparing product formation using the original 5-prime oligo (first detection oligonucleotide) of 87 nucleotides with each shortened version of the 3′ oligo (third detection oligonucleotide). Table 10 shows qPCR Ct values for 3-marker combinations, demonstrating no difference in product expression in Expi293 cells between 3′ oligos, and no change in background signal in PBS.

TABLE 10

| | | qPCR Ct values (duplicate) | | | | | | % CV (Linear trasformed) | | | | | |
|---|---|---|---|---|---|---|---|---|---|---|---|---|---|
| | | 3Prime | | | | | | | | | | | |
| | | | | 3 ntGap | 2 ntGap | 1 ntGap | 0 ntGap | | | | | | |
| | | 87 nt | 87 nt | (60 nt) | (59 nt) | (58 nt) | (57 nt) | 87 nt | 87 nt | 3 ntGap | 2 ntGap | 1 ntGap | 0 ntGap |
| | | SD Primer | | | | | | | | | | | |
| | | 16 nt | None | 16 nt | 16 nt | 16 nt | 16 nt | 16 nt | None | 16 nt | 16 nt | 1 6nt | 16 nt |
| Expi293 | Long 5Prime | 14.5 | 17.5 | 14.3 | 14.1 | 14.2 | 14.3 | 0% | 5% | 8% | 9% | 6% | 3% |
| 2.5× | Short 5Prime | 13.6 | 16.6 | 13.5 | 13.5 | 13.5 | 13.5 | 3% | 1% | 5% | 1% | 1% | 11% |
| PBS | 64 nt 5Prime | 32.9 | 31.9 | 30.6 | 31.3 | 30.8 | 30.9 | 30% | 23% | 18% | 15% | 9% | 3% |
| | 41 nt 5Prime | 30.4 | 30.5 | 28.9 | 29.4 | 29.7 | 29.6 | 3% | 11% | 52% | 16% | 22% | 6% |

The original design of the first and second conjugating oligonucleotides (“Antibody-Anchor” or “detector anchor” in FIG. 17) was 40 nucleotides long, consisting of 20 nucleotides for hybridization with both the 3′ Prime (third detection oligonucleotide) and Splint oligo (second detection oligonucleotide), and an additional 20 nucleotides on the 5′ end to provide structural flexibility. This design was modified by removing the 20 nucleotides at the 5′ end, thereby shortening the detector anchor to 20 nucleotides. The effects of this modification were evaluated using marker combinations including CD81—to represent an abundant target—and CD44—to represent a low-abundance target—on unmodified Expi293 EV samples. Table 11 shows qPCR Ct values for full-length 3-marker combinations, demonstrating a slight increase in specific product formation in Expi293 EVs with the shortened detector anchor, and no change in background signal in PBS controls.

TABLE 11

| | | | qPCR Ct - Average of two replicates | | | | | | | |
|---|---|---|---|---|---|---|---|---|---|---|
| | | | Splint | | | | | | | |
| | | | CD81 | | | | CD44 | | | |
| | | | Short Anchor | | Long Anchor | | Short Anchor | | Long Anchor | |
| | | | Expi293 | PBS | Expi293 | PBS | Expi293 | PBS | Expi293 | PBS |
| CD81 | CD81 | Short | 12.7 | 30.4 | 12.8 | 30.9 | 17.6 | 32.6 | 19.9 | 32.5 |
| | | Anchor | | | | | | | | |
| | | Long | 14.0 | 28.5 | 14.4 | 32.7 | 15.6 | 30.1 | 20.8 | 33.2 |
| | | Anchor | | | | | | | | |
| | CD44 | Short | 20.0 | 33.2 | 18.0 | 33.1 | 24.0 | 35.7 | 25.9 | 35.8 |
| | | Anchor | | | | | | | | |
| | | Long | 22.5 | 30.7 | 22.5 | 35.8 | 26.1 | 31.9 | 28.7 | 36.9 |
| | | Anchor | | | | | | | | |
| CD44 | CD81 | Short | 17.7 | 32.0 | 17.9 | 32.5 | 23.4 | 33.5 | 26.6 | 33.1 |
| | | Anchor | | | | | | | | |
| | | Long | 19.1 | 30.1 | 19.4 | 32.5 | 20.7 | 32.0 | 27.5 | 33.7 |
| | | Anchor | | | | | | | | |
| | CD44 | Short | 29.3 | 35.5 | 23.8 | 34.5 | 32.6 | 36.2 | 33.9 | 34.4 |
| | | Anchor | | | | | | | | |
| | | Long | 30.0 | 31.1 | 30.8 | 35.8 | 32.3 | 32.4 | 35.8 | 38.5 |
| | | Anchor | | | | | | | | |

The specificity of the signal generated by PLLSD using the optimized, shortened oligonucleotides as described in FIG. 17 is illustrated in Table 12. The specificity of the signal generated by PLLSD using oligo as described in FIG. 17 was tested by replacing the capture surface marker CD81, or the splint surface marker CD81, or the 3-prime surface marker CD81 by an isotope control (IgG). The specificity was also tested by removing the capture surface marker, or the splint surface marker, or the 3-prime surface marker from the mix of PLLSD reagents (none). The signal decreased at least 30-fold when surface marker CD81 was replaced by an isotope control.

TABLE 12

| | | | | qPCR data (triplicates) | | | |
|---|---|---|---|---|---|---|---|
| | Capture | Splint | 3Prime | ExPi | | PBS | |
| Enzyme | Ab | Ab | Ab | Avg Ct | % CV | Avg Ct | % CV |
| Ligase | CD81 | CD81 | CD81 | 12.1 | 4% | 30.0 | 23% |
| | None | CD81 | CD81 | 28.1 | 17% | 32.6 | 24% |
| | IgG | CD81 | CD81 | 30.5 | 16% | 32.4 | 10% |
| | CD81 | None | CD81 | 20.0 | 6% | 33.8 | 42% |
| | CD81 | IgG | CD81 | 17.2 | 2% | 30.2 | 14% |
| | CD81 | CD81 | None | 25.2 | 11% | UND | NA |
| | CD81 | CD81 | IgG | 22.5 | 8% | 24.7 | 7% |
| | IgG | IgG | IgG | 28.6 | 5% | 28.3 | 15% |
| No | CD81 | CD81 | CD81 | 25.5 | 6% | 35.0 | 20% |
| Ligase | None | CD81 | CD81 | 34.1 | 2% | 34.4 | 38% |
| | IgG | CD81 | CD81 | 34.5 | 31% | 34.5 | 16% |
| | CD81 | None | CD81 | 25.0 | 2% | 34.6 | 23% |
| | CD81 | IgG | CD81 | 25.2 | 7% | 33.8 | 30% |
| | CD81 | CD81 | None | 26.3 | 4% | 36.4 | 23% |
| | CD81 | CD81 | IgG | 26.4 | 11% | 33.1 | 43% |
| | IgG | IgG | IgG | 33.9 | 78% | 33.6 | 31% |

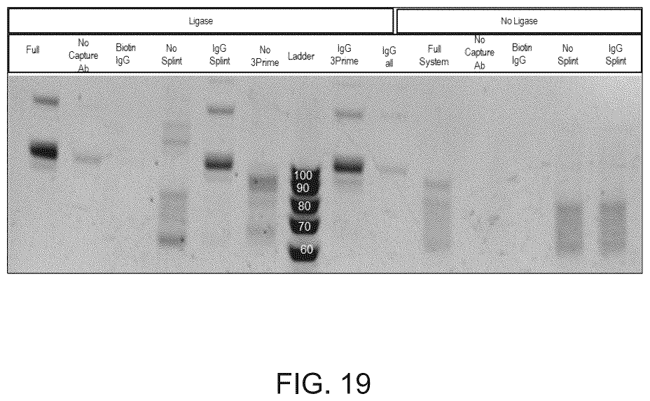

The full-length specific signal was knocked-off when surface marker CD81 was removed from the reagents, as illustrated by the gel image corresponding to the qPCR Ct in FIG. 19.

### Example 4: Use of PESD Constructs and Methods for 2-Marker EV Combinatorial Immunosequencing

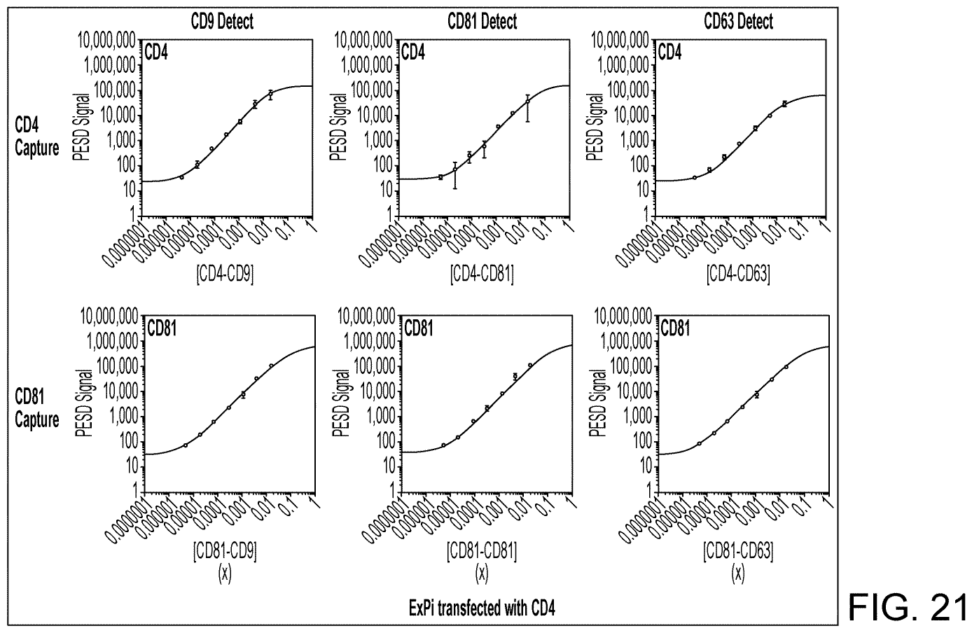

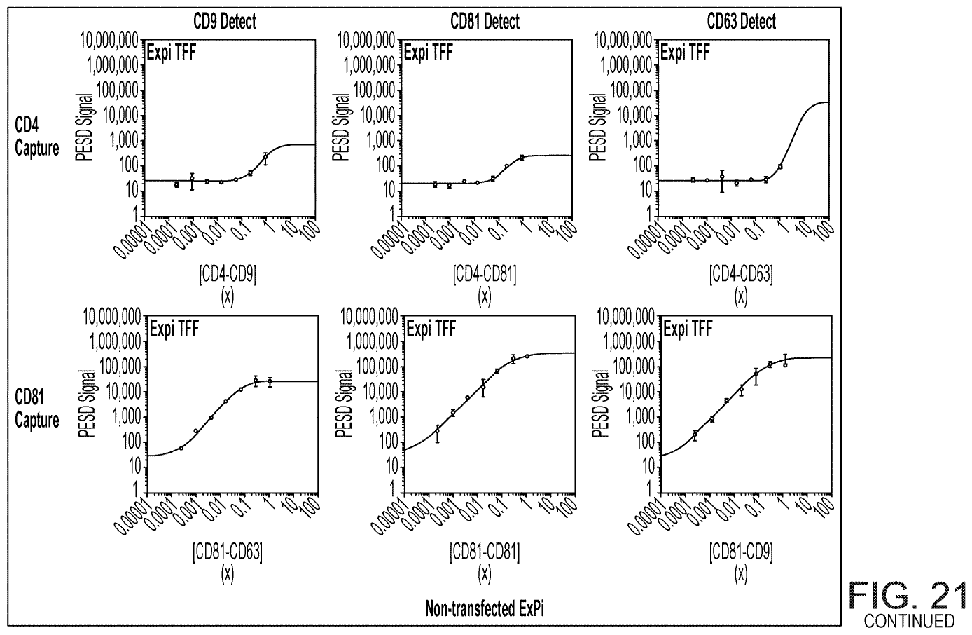

FIGS. 20A and 20B illustrate the use of the Proximity Extension Strand Displacement (PESD) constructs and assay methods for the capture and detection of intact EVs, employing buffer and antibody diluent conditions specifically adapted to preserve the integrity of the EVs lipid bilayer. This assay is designed to enable efficient, high-throughput screening of EV samples in a two-marker combinatorial format. FIG. 21 shows an experiment using common EV markers CD81, CD9 and CD63 as detectors, in combination with CD4 and CD81 as capture markers. Combinations including CD4 yielded signals proportional to EV concentration in samples from Expi293 cells transfected with CD4, but not EV samples from untransfected Expi293 cells, which do not inherently express CD4, demonstrating the specificity of the assay. This assay format can be expanded to screen all combinations of hundreds of capture markers with hundreds of detector markers in a single reaction. As in PESD assays for protein biomarkers or PESD assays for antibody screening, the identity of the capture and detector antibodies binding to each EV would be identified by sequencing the barcodes shown in FIG. 20 by NGS.

### Example 5: Adapters to Enable Common Reagent Set for PLLSD, PESD and Other Assay Formats

It would be useful to produce a single detector antibody/anchor oligo conjugate that could be used in any of the combinatorial immuno-sequencing embodiments described above as well as in PESD, and in ECL assays, including ultrasensitive EV assays. This is accomplished by use of adapter oligonucleotides to bridge between the antibody/anchor oligo conjugate and the other functional oligonucleotides required for each assay format.

In the case of PLLSD, an option is to use the PESD forward anchor (conjugating oligonucleotide) in lieu of the existing first or second conjugating oligonucleotide (3-prime/splint PLLSD Detector Anchor), as illustrated in FIG. 22A. This requires modification of the 3-prime oligonucleotide sequence by replacing the second primer site with a sequence complementary to the PESD forward anchor. This also requires a change in the orientation of the splint oligonucleotide for compatibility with the 3′thiol end of the PESD forward anchor. This flipped orientation, however, can be suboptimal due to length and steric constraints.

The change in orientation can be avoided by the addition of an adaptor sequence, complementary to both the existing 3-prime/splint PLLSD Detector Anchor and to the PESD forward anchor. This keeps both the 3-prime and splint oligonucleotide sequences unchanged and enables use of the PESD forward anchor in conjunction with an optimized version of the adaptor sequence, as illustrated in FIG. 22B.

The PLLSD detector anchor may also be used across several immunoassay platforms, with appropriate adapter oligonucleotides. For ECL immunoassays an adaptor oligonucleotide sequence is designed with one region complementary to the PLLSD Detector sequence, and another region is linked to a SULFO-TAG-linked molecule for ECL detection. For ultrasensitive EV assays, the adaptor oligonucleotide sequence contains one region complementary to the PLLSD Detector and a second region corresponding the sequences of the proximity probes “PP1” (first nucleic acid probe) or “PP2” (second nucleic acid probe), which are used as detectors in ultrasensitive EV assays as exemplified in WO 2015/175856.

1. A method of determining surface markers of a surface marker displaying agent (SMDA) comprising:
(A) contacting the SMDA with (i) a capture reagent bound to a surface, (ii) a plurality of binding reagents, and (iii) an oligonucleotide insert,
wherein the plurality of binding reagents comprises:
a) a first binding reagent comprising a first detection sequence that comprises (i) a first hybridization sequence, wherein at least a portion of the first hybridization sequence is complexed with a first blocker oligonucleotide, (ii) a first primer site, and (iii) a first barcode sequence;
b) a second binding reagent comprising a second detection sequence that comprises (i) a second hybridization sequence, (ii) a third hybridization sequence comprising a second barcode sequence, and (iii) a fourth hybridization sequence; and
c) a third binding reagent comprising a third detection sequence that comprises (i) a fifth hybridization sequence, wherein at least a portion of the fifth hybridization sequence is complexed with a second blocker oligonucleotide, (ii) a second primer site, and (iii) a third barcode sequence,
wherein the first hybridization sequence and the second hybridization sequence are complementary;
wherein the fourth hybridization sequence and the fifth hybridization sequence are complementary;
wherein the third hybridization sequence is complementary to the oligonucleotide insert;
(B) generating an output oligonucleotide by ligating a hybridized first detection sequence to a hybridized oligonucleotide insert, and ligating the hybridized oligonucleotide insert to the hybridized third detection sequence to form a single-strand output oligonucleotide,
wherein when at least three binding reagents bind to surface markers of the SMDA, the output oligonucleotide generated comprises the first barcode sequence, the second barcode sequence and the third barcode sequence; and
(C) sequencing the output oligonucleotide to identify the surface markers of the SMDA.

2.-9. (canceled)

10. The method of claim 1, wherein (i) one or more of the first and second blocker oligonucleotide comprises a 3′ overhang; (ii) one or more of the first and second blocker oligonucleotide is about 5 to about 20 nucleotides in length; (iii) the oligonucleotide insert is about 10 to about 30 nucleotides in length; (iv) one or more of the first, second and third detection sequences is about 20 to about 50 nucleotides in length; or (v) any combination of (i) to (iv).

11.-17. (canceled)

18. The method of claim 1, wherein one or more of the capture reagent, the first binding reagent, the second binding reagent, and the third binding reagent:
(i) binds to a surface marker selected from: CD9, CD73, CD324, CD325, CD326, CD13, CD66a, CD66e, CD10, CD31, CD36, CD141, CD14, CD54, CD26, MHC Class II molecules, FLT1, and any combination thereof;
(ii) comprises an antibody or antigen binding fragment thereof, antigen, ligand, receptor, oligonucleotide, hapten, epitope, mimitope, lipid binding protein, carbohydrate binding protein, DNA aptamer or RNA aptamer; or
(iii) both (i) and (ii).

19.-22. (canceled)

23. A method of determining surface markers of a surface marker displaying agent (SMDA) comprising:
(A) contacting the SMDA with (i) a capture reagent bound to a surface, (ii) a plurality of binding reagents, and (iii) an oligonucleotide insert,
wherein the plurality of binding reagents comprises:
a) a first binding reagent comprising a first conjugating oligonucleotide and a first detection sequence, wherein the first detection sequence comprises: (i) a first hybridization sequence, (ii) a first barcode sequence, (iii) a first primer site, and (iv) a first connecting sequence complementary to the first conjugating oligonucleotide;
b) a second binding reagent comprising a second conjugating oligonucleotide and a second detection sequence, wherein the second detection sequence comprises: (i) a second hybridization sequence, (ii) a third hybridization sequence comprising a second barcode sequence, (iii) a fourth hybridization sequence, and (iv) and a second connecting sequence complementary to the second conjugating oligonucleotide; and
c) a third binding reagent comprising a third conjugating oligonucleotide and a third detection sequence, wherein the third detection sequence comprises: (i) a fifth hybridization sequence, (ii) a third barcode sequence, (iii) a second primer site, (iv) a third connecting sequence complementary to the third conjugating oligonucleotide, and (v) a third primer site,
wherein the first hybridization sequence and the second hybridization sequence are complementary;
wherein the fourth hybridization sequence and the fifth hybridization sequence are complementary; and
wherein the third hybridization sequence is complementary to the oligonucleotide insert;
(B) generating an output oligonucleotide by ligating the hybridized first detection sequence to the hybridized oligonucleotide insert, and ligating the hybridized oligonucleotide insert to the hybridized third detection sequence to form a single-strand output oligonucleotide,
wherein when at least three binding reagents bind to three surface markers of the SMDA, the output oligonucleotide generated comprises the first barcode sequence, the second barcode sequence and the third barcode sequence; and
(C) sequencing the output oligonucleotide to identify the three surface markers of the SMDA.

24. The method of claim 23, wherein (i) at least a portion of the first detection sequence is complexed with a first blocker oligonucleotide, (ii) at least a portion of the third detection sequence is complexed with a second blocker oligonucleotide, or (iii) both (i) and (ii).

25.-29. (canceled)

30. A method of determining surface markers of a surface marker displaying agent (SMDA), comprising:
(A) contacting a sample comprising a SMDA with:
(a) an anchoring reagent hybridized to a first detection oligonucleotide, wherein the first detection oligonucleotide comprises a first unique barcode sequence and a first primer site;
(b) a first binding reagent hybridized to a second detection oligonucleotide, wherein the second detection oligonucleotide comprises a second unique barcode sequence,
wherein the first detection oligonucleotide and the second detection oligonucleotide comprise complementary nucleotide sequences;
(c) a second binding reagent hybridized to a third detection oligonucleotide, wherein the third detection oligonucleotide comprises a third unique barcode sequence, a second primer site, and a third primer site,
wherein the second detection oligonucleotide and the third detection oligonucleotide comprise complementary nucleotide sequences;
(d) a capture reagent; and
(e) an oligonucleotide insert complementary to the second detection oligonucleotide,
wherein if (i) the SMDA binds to the capture reagent, the first and second binding reagents, and (ii) the first detection oligonucleotide hybridizes to the second detection oligonucleotide, the second detection oligonucleotide hybridizes to the third detection oligonucleotide, and the oligonucleotide insert hybridizes to the second detection oligonucleotide, then a single-strand output oligonucleotide is generated that comprises the first, second, and third unique barcode sequences;
(B) amplifying the single-strand output oligonucleotide; and
(C) sequencing the amplified output oligonucleotide to identify the first, second, and third unique barcode sequences, thereby determining at least three unique surface markers of the SMDA.

31. The method of claim 30, wherein (i) at least a portion of the first detection oligonucleotide is hybridized to a first blocker oligonucleotide; (ii) at least a portion of the third detection oligonucleotide is hybridized to a second blocker oligonucleotide; or (iii) both (i) and (ii).

32.-50. (canceled)

51. The method of claim 30, wherein: (i) the first detection oligonucleotide comprises a connecting sequence complementary to a conjugating oligonucleotide on the anchoring reagent: (ii) the second detection oligonucleotide comprises a connecting sequence complementary to a conjugating oligonucleotide on the first binding reagent: (iii) the third detection oligonucleotide comprises a connecting sequence complementary to a conjugating oligonucleotide on the second binding reagent; or (iv) any combination of (i) to (iii).

52.-58. (canceled)

59. The method of claim 1, wherein the method further comprises;
(i) a pre-treatment step to deplete platelet-derived EVs using anti-CD41 and anti-CD61 beads;
(ii) a washing step with a washing solution, wherein the washing solution comprises arginine;
(iii) adding a hairpin blocker to inhibit inter-EV ligation; or
(iv) any combination of (i) to (iii).

60.-62. (canceled)

63. A method for detecting colorectal tumor-derived surface marker displaying agent (SMDA) in a sample from a subject suspected of having a colorectal tumor or suspected of having colorectal cancer (CRC), comprising detecting at least two CRC-related markers on the SMDA in the sample.

64. A method of (i) determining eligibility of a subject to participate in a clinical trial of a therapeutic drug for preventing or delaying colorectal cancer (CRC), (ii) conducting a clinical trial of a therapeutic drug or intervention for CRC, (iii) distinguishing a subject afflicted with CRC from an individual not afflicted with CRC, or (iv) treating CRC in a subject in need thereof,
comprising:
(a) obtaining a measurement of at least two CRC-related surface marker levels on colorectal tumor-derived surface marker displaying agents (SMDAs) in a biological sample from the subject;
(b) (i) determining the eligibility of the subject for the clinical trial based on the measurement of the at least two CRC-related surface marker levels; or (ii) identifying, based on the measurement of the at least two CRC-related surface marker levels, the subject as afflicted with CRC; and
(c) conducting a clinical trial of a therapeutic drug or intervention for CRC, treating CRC, or administering the therapeutic drug to the subject.

65.-67. (canceled)

68. A method of monitoring response to treatment for colorectal cancer (CRC) in a subject, the method comprising:
(a) determining, based on a first measurement of at least two CRC-related marker levels on colorectal tumor-derived surface marker displaying agents (SMDAs) in a biological sample from the subject, wherein the first measurement is obtained prior to administration of a treatment regimen for CRC and a second measurement of the at least two CRC-related marker levels in the subject at one or more time points after administration of the treatment regimen for CRC has been initiated, that the subject is responding positively to the CRC treatment regimen, and
(b) continuing to administer the treatment regimen for CRC to the subject.

69. A method of identifying colorectal cancer (CRC) in a human, the method comprising:
obtaining measured levels of at least two CRC-related marker levels on colorectal tumor-derived surface marker displaying agents (SMDAs) in a biological sample suspected of containing SMDAs from the human,
wherein the sample is selected from the group consisting of whole blood, serum, plasma, and combinations thereof, and
wherein the levels of the at least two CRC-related markers are obtained by a multimarker immunoassay comprising contacting the sample with a first oligonucleotide-conjugated capture entity that binds a first CRC-related surface marker of the SMDA, a second oligonucleotide-conjugated splint entity that binds a second CRC-related surface marker of the SMDA, a third oligonucleotide-conjugated staple entity that binds a common marker or a third CRC-related surface marker of the SMDA, and a surface.

70. The method of claim 63, wherein the at least two CRC-related surface markers comprise any combination selected from: CD73, CD324, CD325, CD326, CD13, CD66e, CD66a, CD10, CD31, CD36, CD141, CD14, and CD54.

71.-83. (canceled)

84. A construct comprising:
(a) a capture reagent,
(b) at least three unique binding reagents and an oligonucleotide insert, and
(c) at least two blocker oligonucleotides,
wherein each unique binding reagent comprises a detection sequence comprising a unique barcode oligonucleotide sequence;
wherein the at least three binding reagents comprises:
a) a first binding reagent comprising a first detection sequence that comprises a (i) first hybridization sequence, wherein at least a portion of the first hybridization sequence is complexed with a first blocker oligonucleotide, and (ii) a first primer site;
b) a second binding reagent comprising a second detection sequence that comprises (i) a second hybridization sequence, (ii) a third hybridization sequence, and (iii) a fourth hybridization sequence; and
c) a third binding reagent comprising a third detection sequence that comprises Gii a fifth hybridization sequence, wherein at least a portion of the fifth hybridization sequence is complexed with a second blocker oligonucleotide, and (ii) a second primer site,
wherein the first hybridization sequence and the second hybridization sequence are complementary;
wherein the fourth hybridization sequence and the fifth hybridization sequence are complementary; and
wherein the third hybridization sequence is complementary to the oligonucleotide insert.

85.-88. (canceled)

89. A method of determining surface markers of a surface marker displaying agent (SMDA), comprising:
(A) contacting a sample comprising an SMDA with:
(a) a capture reagent that binds to a first surface marker of the SMDA;
(b) an anchoring reagent comprising an anchoring oligonucleotide;
(c) a first detection oligonucleotide comprising: (i) a first primer site; (ii) a first barcode sequence; (iii) a first blocker complement sequence; and (iv) a first hybridization sequence,
wherein the first detection oligonucleotide and the anchoring oligonucleotide comprise complementary nucleotide sequences;
(d) a first binding reagent that binds to a second surface marker of the SMDA, wherein the first binding reagent comprises a first conjugating oligonucleotide;
(e) a second detection oligonucleotide comprising: (i) a second hybridization sequence; (ii) a third hybridization sequence comprising a second barcode sequence; and (iii) a fourth hybridization sequence;
(f) a second binding reagent that binds to a third surface marker of the SMDA, wherein the second binding reagent comprises a second conjugating oligonucleotide;
(g) a third detection oligonucleotide comprising: (i) a fifth hybridization sequence; (ii) a second blocker complement sequence; (iii) a third barcode sequence; (iv) a second primer site; and (iv) a third primer site; and
(h) an oligonucleotide insert,
wherein the first hybridization sequence and the second hybridization sequence are complementary;
wherein the fourth hybridization sequence and the fifth hybridization sequence are complementary; and
wherein the third hybridization sequence is complementary to the oligonucleotide insert;
(B) generating an output oligonucleotide by ligating the hybridized first detection oligonucleotide to the hybridized oligonucleotide insert, and ligating the hybridized oligonucleotide insert to the hybridized third detection oligonucleotide to form an output oligonucleotide,
wherein the output oligonucleotide generated comprises the first barcode sequence, the second barcode sequence, and the third barcode sequence;
(C) amplifying the output oligonucleotide; and
(D) sequencing the output oligonucleotide to identify the first, second, and third barcode sequences, thereby determining the surface markers of the SMDA.

90. The method of claim 89, wherein: (i) the second detection oligonucleotide and the first conjugating oligonucleotide comprise complementary nucleotide sequences; (ii) the third detection oligonucleotide and the second conjugating oligonucleotide comprise complementary nucleotide sequences; (iii) or both (i) and (ii).

91. (canceled)

92. The method of claim 89, further comprising contacting the sample with:
(i) a first adaptor oligonucleotide, wherein the first adaptor oligonucleotide comprises: (a) a first sequence complementary to the first conjugating oligonucleotide, (b) a second sequence complementary to a 3′ sequence of the second detection oligonucleotide, or (c) both (a) and (b);
(ii) a second adaptor oligonucleotide, wherein the second adaptor oligonucleotide comprises: (a) a first sequence complementary to the second conjugating oligonucleotide, (b) a second sequence complementary to the second primer site, or (c) both (a) and (b); or
(iii) both (i) and (ii).

93-102. (canceled)

103. The method of claim 89, wherein (i) the first blocker complement sequence and at least a portion of the first hybridization sequence are hybridized to a first blocker oligonucleotide; (ii) the second blocker complement sequence and at least a portion of the fifth hybridization sequence are hybridized to a second blocker oligonucleotide; or (iii) both (i) and (ii).

104.-122. (canceled)

123. A method of determining surface markers of a surface marker displaying agent (SMDA) comprising:
(A) contacting the SMDA with:
(a) a capture reagent that binds to a first surface marker of the SMDA;
(b) an anchoring reagent comprising a first hybridization sequence;
(c) a reverse oligonucleotide comprising: (i) a first barcode sequence; (ii) a second hybridization sequence; and (iii) a third hybridization sequence,
(d) a binding reagent that binds to a second surface marker of the SMDA, wherein the binding reagent comprises a fourth hybridization sequence; and
(e) a forward oligonucleotide comprising: (i) a fifth hybridization sequence; (ii) a second barcode sequence; and (iii) a sixth hybridization sequence,
wherein the first hybridization sequence and the second hybridization sequence are complementary;
wherein the third hybridization sequence and the sixth hybridization sequence are complementary; and
wherein the fourth hybridization sequence and the fifth hybridization sequence are complementary;
B) generating an output oligonucleotide by extending and ligating the hybridized reverse oligonucleotide and the hybridized forward oligonucleotide to form an output oligonucleotide,
wherein the output oligonucleotide generated comprises the first barcode sequence and the second barcode sequence;
C) amplifying the output oligonucleotide; and
D) sequencing the output oligonucleotide to identify the first and second barcode sequences, thereby determining the surface markers of the SMDA.

124. The method of claim 123, wherein the reverse oligonucleotide, the forward oligonucleotide or both are hybridized with a blocker oligonucleotide prior to hybridization of the third hybridization sequence to the sixth hybridization sequence.

125. The method of claim 123, wherein the capture reagent is bound to a surface.

126. (canceled)

127. (canceled)

128. The method of claim 123, wherein the capture reagent, the binding reagent, or both comprise an antibody or antigen binding fragment thereof.

129.-135. (canceled)

136. A kit for determining surface markers of a surface marker displaying agent (SMDA), for identifying SMDAs that harbor combinations of surface markers, for detecting populations of SMDAs having certain surface markers, and/or for detecting or quantifying multiple populations of SMDAs where each population has a specific set of surface markers, the kit comprising, in one or more vials, containers, or compartments:
(A) (a) a capture reagent;
(b) an anchoring reagent comprising an anchoring oligonucleotide;
(c) a first binding reagent comprising a first conjugating oligonucleotide;
(d) a second binding reagent comprising a second conjugating oligonucleotide;
(e) a first detection oligonucleotide comprising: (i) a first primer site; (ii) a first barcode sequence; (iii) a first blocker complement sequence; and (iv) a first hybridization sequence,
wherein the first detection oligonucleotide and the anchoring oligonucleotide comprise complementary nucleotide sequences;
(f) a second detection oligonucleotide comprising: (i) a second hybridization sequence; (ii) a third hybridization sequence comprising a second barcode sequence; and (iii) a fourth hybridization sequence;
(g) a third detection oligonucleotide comprising: (i) a fifth hybridization sequence; (ii) a second blocker complement sequence; (iii) a third barcode sequence; (iv) a second primer site; and (iv) a third primer site; and
(h) an oligonucleotide insert,
wherein the first hybridization sequence and the second hybridization sequence are complementary;
wherein the fourth hybridization sequence and the fifth hybridization sequence are complementary; and
wherein the third hybridization sequence is complementary to the oligonucleotide insert; or
(B) (a) a capture reagent,
(b) at least three unique binding reagents and an oligonucleotide insert, and
(c) at least two blocker oligonucleotides,
wherein each unique binding reagent comprises a detection sequence comprising a unique barcode oligonucleotide sequence;
wherein the at least three unique binding reagents comprises:
a) a first binding reagent comprising a first detection sequence that comprises a (i) first hybridization sequence, wherein at least a portion of the first hybridization sequence is complexed with a first blocker oligonucleotide, and (ii) a first primer site;
b) a second binding reagent comprising a second detection sequence that comprises i) a second hybridization sequence, (ii) a third hybridization sequence, and (iii) a fourth hybridization sequence; and
c) a third binding reagent comprising a third detection sequence that comprises a fifth hybridization sequence, wherein at least a portion of the fifth hybridization sequence is complexed with a second blocker oligonucleotide, and (ii) a second primer site,
wherein the first hybridization sequence and the second hybridization sequence are complementary;
wherein the fourth hybridization sequence and the fifth hybridization sequence are complementary; and
wherein the third hybridization sequence is complementary to the oligonucleotide insert.

137. (canceled)

138. (canceled)

139. The kit of claim 136, further comprising:
(a) a first adaptor oligonucleotide, wherein the first adaptor oligonucleotide comprises (i) a first sequence complementary to at least a portion of the second detection oligonucleotide; (ii) a second region complementary to the first conjugating oligonucleotide; or both (i) and (ii);
(b) a second adaptor oligonucleotide, wherein the second adaptor oligonucleotide comprises (i) a first region complementary to at least a portion of the third detection oligonucleotide; (ii) a second region complementary to the second conjugating oligonucleotide; or both (i) and (ii);
(c) at least two blocker oligonucleotides, wherein (i) at least a portion of the first hybridization sequence is complexed or capable of being complexed with a first blocker oligonucleotide: (ii) at least a portion of the fifth hybridization sequence is complexed or capable of being complexed with a second blocker oligonucleotide; or (iii) both (i) and (ii);
(d) a strand-displacement primer and strand-displacement polymerase;
(e) a wash buffer comprising arginine;
(f) a surface;
(g) a forward primer, a reverse primer, or both;
(h) a DNA polymerase;
(i) a ligase;
(i) a hairpin blocker to inhibit inter-EV ligation; or
(k) any combination of (a) to (j).

140.-152. (canceled)
